(19) United States
(12) Patent Application Publication
(10) Pub. No.: US 2023/0049217 A1
(43) Pub. Date: Feb. 16, 2023

(54) COMPOSITIONS AND METHODS FOR ENHANCING VISUAL FUNCTION

(71) Applicant: Novartis AG, Basel (CH)

(72) Inventors: Karen I. Guerin, Braintree, MA (US); Robert M. Kotin, Cambridge, MA (US); Eric Pastor, Newton, MA (US); Xiaozhi Ren, Brighton, MA (US); Sharon Young, Fremont, CA (US)

(73) Assignee: Novartis AG, Basel (CH)

(21) Appl. No.: 17/747,881

(22) Filed: May 18, 2022

Related U.S. Application Data
(60) Provisional application No. 63/191,525, filed on May 21, 2021.
(60) Provisional application No. 63/292,746, filed on Dec. 22, 2021.

(51) Int. Cl. A61K 38/17; C12N 15/86; A61K 48/00; A61P 27/02

(52) U.S. Cl. CPC A61K 38/177; C12N 15/86; A61K 48/0058; A61K 48/0066; A61P 27/02; C12N 2750/14122; C12N 2750/14145; C12N 2750/14143; C12N 2750/14152

(57) ABSTRACT

The present disclosure provides compositions and methods of restoring or enhancing visual function in an individual by administering to the individual a pharmaceutical composition comprising a recombinant adeno-associated viral (rAAV) vector having a polynucleotide sequence that encodes a medium wavelength cone opsin (MW-opsin). The MW-opsin is expressed in a retinal cell in the individual, thereby restoring or enhancing visual function.

### BACKGROUND

Inherited and age-related retinal degenerative diseases cause progressive loss of rod and cone photoreceptors, leading to blindness. Despite loss of the light-sensing cells required for vision, downstream neurons of the inner retina survive in a functional state, providing a target for optogenetic therapy. Optogenetic approaches have encountered certain limitations, including: a) very low light sensitivity in microbial opsins and chemically engineered mammalian receptors; b) very slow kinetics in retinal opsins; and c) a lack of the mechanisms of adaptation that provide natural vision with high sensitivity across a very wide range of ambient light levels.

Thus, there is a need in the art for improved optogenetic approaches to treating ocular disorders.

### SUMMARY

In one aspect of the invention, the present disclosure provides a recombinant expression vector comprising a first inverted terminal repeat (ITR) polynucleotide sequence, a promoter polynucleotide sequence operatively linked to a polynucleotide sequence encoding a medium-wavelength cone opsin (MW-opsin) transgene, a polyA polynucleotide sequence, and a second ITR polynucleotide sequence. In another aspect, the present disclosure provides a recombinant expression vector comprising a first inverted terminal repeat (ITR) polynucleotide sequence, a promoter polynucleotide sequence operatively linked to a polynucleotide sequence encoding a medium-wavelength cone opsin (MW-opsin) transgene, an enhancer polynucleotide sequence, a polyA polynucleotide sequence, and a second ITR polynucleotide sequence. In another aspect, the present disclosure provides a recombinant expression vector comprising a first inverted terminal repeat (ITR) polynucleotide sequence, a promoter polynucleotide sequence operatively linked to a polynucleotide sequence encoding a medium-wavelength cone opsin (MW-opsin) transgene, a polyA polynucleotide sequence, an intron polynucleotide sequence, and a second ITR polynucleotide sequence. In another aspect, the present disclosure provides a recombinant expression vector comprising a first inverted terminal repeat (ITR) polynucleotide sequence, a promoter polynucleotide sequence operatively linked to a polynucleotide sequence encoding a medium-wavelength cone opsin (MW-opsin) transgene, an enhancer polynucleotide sequence, a polyA polynucleotide sequence, an intron polynucleotide sequence, and a second ITR polynucleotide sequence.

In one embodiment, the first ITR polynucleotide sequence comprises the sequence of SEQ ID NO: 1. In one embodiment, the promoter polynucleotide sequence comprises the sequence of SEQ ID NO: 2. In on embodiment, the polynucleotide sequence encoding a MW-opsin transgene has been codon-optimized so that the MW-opsin protein translated from the MW-opsin transgene is produced in larger quantities as compared to the wild-type MW-opsin transgene. In one embodiment, the polynucleotide sequence encoding a MW-opsin transgene comprises a sequence that is 85% identical to the sequence of SEQ ID NO: 3. In one embodiment, the polynucleotide sequence encoding a MW-opsin transgene comprises a sequence that is 90% identical to the sequence of SEQ ID NO: 3. In one embodiment, the polynucleotide sequence encoding a MW-opsin transgene comprises the sequence of SEQ ID NO: 3. In one embodiment, the enhancer polynucleotide sequence comprises the sequence of SEQ ID NO: 4. In one embodiment, the polyA polynucleotide sequence comprises the sequence of SEQ ID NO: 5. In one embodiment, the intron polynucleotide sequence comprises the sequence of SEQ ID NO: 6. In one embodiment, the second ITR polynucleotide sequence comprises the sequence of SEQ ID NO: 7. In one embodiment, the recombinant expression vector further comprises a polynucleotide sequence conferring resistance to an antibiotic. In a specific embodiment, the antibiotic is kanamycin. In a specific embodiment, the recombinant expression vector comprises the sequence of SEQ ID NO: 8. In a specific embodiment, the recombinant expression vector comprises the sequence of SEQ ID NO: 9.

In one embodiment, the recombinant expression vector is a recombinant viral vector. In specific embodiments, the recombinant viral vector is an adeno-associated viral vector, a lentiviral vector, a herpes simplex vector, or a retroviral vector. In a specific embodiment, the recombinant viral vector is an adeno-associated viral vector. In a further specific embodiment, the recombinant viral vector is AAV2. In a further embodiment, the recombinant adeno-associate viral vector comprises a nucleotide sequence encoding a variant capsid polypeptide that confers increased infectivity of a retinal cell and/or confers increased ability to cross the inner limiting membrane, as compared to a wild-type adeno-associated viral capsid. In a further embodiment, the variant capsid polypeptide has a sequence selected from the group consisting of SEQ ID NOs: 10-197. In a further embodiment, the variant capsid polypeptide has a sequence selected from the group consisting of SEQ ID NOs: 10-20. In a further embodiment, the variant capsid polypeptide has the sequence of SEQ ID NO: 14. In a further embodiment, the variant capsid polypeptide has the sequence of SEQ ID NO: 15. In a further embodiment, the variant capsid polypeptide has the sequence of SEQ ID NO: 16.

In another aspect of the invention, the present disclosure provides a method of restoring or enhancing visual function in an individual comprising administering to the individual the recombinant expression vector described above, wherein the administering provides for expression of the MW-opsin transgene in a retinal cell in the individual and restoration or enhancement of visual function. In one embodiment, expression of the MW-opsin transgene in the retinal cell provides for patterned vision and image recognition by the individual. In a further embodiment, the image recognition is of a static image or a pattern. In still a further embodiment, the image recognition is of a moving image or a pattern. In one embodiment, the expression of the MW-opsin transgene in the retinal cell provides for distinguishing between an image comprising a vertical line and an image comprising a horizontal line in a spatial pattern discrimination assay. In another embodiment, the expression of the MW-opsin transgene in the retinal cell provides for distinguishing between an image comprising a static line and an image comprising a moving line in a spatial pattern discrimination assay. In another embodiment, the expression of the MW-opsin transgene in the retinal cell provides for distinguishing between flashing light and constant light in a temporal light pattern assay. In one embodiment, the expression of the MW-opsin transgene in the retinal cell provides for recognizing an image at a light intensity of from about 10⁴ W/cm² to about 10 W/cm² in an image recognition assay. In one embodiment, the expression of the MW-opsin transgene in the retinal cell provides for distinguishing between an area with white light and an area without white light in a light avoidance assay. In one embodiment, the expression of the MW-opsin transgene in the retinal cell provides for image recognition at a light intensity that is at least 10-fold lower than the light intensity required to provide for image recognition by an individual expression a channelrhodopsin polypeptide in a retinal cell. In one embodiment, the expression of the MW-opsin transgene in the retinal cell provides for kinetics that are at least 2-fold faster than the kinetics conferred on a retinal cell by a rhodopsin polypeptide. In certain embodiments, the administering is via intraocular, intravitreal, or subretinal injection.

In some embodiments, the individual has an ocular disease selected from retinitis pigmentosa, macular degeneration, geographic atrophy, age-related macular degeneration, retinoschisis, Leber's Congenital Amaurosis, cone rod dystrophies, Bardet Biedl syndrome, choroideremia, Usher syndrome, Stargardt disease, and Bietti crystalline dystrophy. In other embodiments, the individual has experienced retinal detachment or photoreceptor loss due to trauma, head injury, or as a complication of another disease (e.g., diabetic retinopathy).

Another aspect of the invention provides a pharmaceutical composition comprising the recombinant expression vector described above and a pharmaceutically acceptable excipient. In an embodiment, the pharmaceutically acceptable excipient comprises saline. In a further embodiment, the composition is sterile.

In another aspect, the present disclosure provides the recombinant expression vector as described above or the pharmaceutical composition as described above for use in treating a subject in need thereof. In one embodiment, the recombinant expression vector as described above or the pharmaceutical composition as described above restores or enhances visual function in a subject. In another aspect, the present disclosure provides the recombinant expression vector as described above or the pharmaceutical composition as described above for use in the manufacture of a medicament, or for use in restoring or enhancing visual function, or for use in the treatment of ocular diseases.

In another aspect, the present disclosure provides a host cell comprising the recombinant expression vector as described above. In another aspect, the present disclosure provides a method of making the recombinant expression vector as described above, wherein the method comprises culturing the host cell described above, lysing the cultured host cells, and extracting and purifying the recombinant expression vector from the lysed cultured host cells. In another aspect, the present disclosure provides a method of making the pharmaceutical composition as described above, wherein the method comprises culturing the host cell as described above, collecting the supernatant of the cultured host cells, concentrating and purifying recombinant viral vectors from the collected supernatant, and adding pharmaceutically acceptable excipients to the purified recombinant viral vectors.

In another aspect, the present disclosure provides a method of treating an ocular disease selected from retinitis pigmentosa, macular degeneration, geographic atrophy, age-related macular degeneration, retinoschisis, Leber's Congenital Amaurosis, cone rod dystrophies, Bardet Biedl syndrome, choroideremia, Usher syndrome, Stargardt disease, or Bietti crystalline dystrophya, wherein the method comprises administering a therapeutically effective amount of the recombinant expression vector as described above or the pharmaceutical composition as described above to a subject in need thereof. In a further embodiment, the ocular disease is retinitis pigmentosa. In yet a further embodiment, the ocular disease is geographic atrophy.

### BRIEF DESCRIPTION OF THE FIGURES

FIG. 1 provides the polynucleotide sequences of SEQ ID NOs: 1-2.

FIG. 2 provides the polynucleotide sequences of SEQ ID NOs: 3-5.

FIG. 3 provides the polynucleotide sequences of SEQ ID NOs: 6-7.

FIG. 4A-4B provides the polynucleotide sequence of SEQ ID NO: 8.

FIG. 5A-5D provides the polynucleotide sequence of SEQ ID NO: 9.

FIG. 6 provides fluorescent images of 293T cells transfected with REV_Kan, and then subjected to immunohistochemistry.

FIG. 7 provides a schematic of the ITR stability evaluation studies.

FIG. 8 provides the imaged agarose electrophoresed gels containing REV_Kan vector after XmaI digestion. When the XmaI restriction site is present in REV_Kan ITRs, DNA fragments having sizes of 3257, 2798, and 1734 bp are observed.

FIG. 9 illustrates exemplary vector transduction in cynomolgus macaque eyes using in situ hybridization with antisense and sense probes. A representative whole slide view of eye sections from one test animal (high dose group, @4.5×10¹¹ vg/eye) with the antisense and sense ISH (black signal) are shown. Note that the antisense ISH signal are present in multifocal areas of central retina, but more uniform distribution in peripheral retina (as indicated by *), ciliary body, iris, iridocorneal angle, lens capsule and optic nerve. Localization pattern of the sense ISH signal was similar to that of antisense ISH. The eye sections showed moderate levels of PPIB (endogenous control) and negative for DapB (a bacterial gene) mRNA signal (not shown).

FIG. 10 illustrates exemplary vector nucleic signal in various anatomical regions of the eye using in situ hybridization with antisense and sense probes. (A) Antisense ISH localization from various regions of eye for a test animal (high dose group, @4.5×1011 vg/eye) is shown. Antisense ISH signal is abundant in macula and in multifocal areas of central retina, particularly within ganglion cells, nerve fiber layer, inner plexiform layer, and inner nuclear layer. Occasionally, antisense ISH signal is detected in outer nuclear layer and photoreceptors. ISH signal was more uniformly detected in peripheral retina. Mild to moderate levels of ISH signal was present in optic nerve, ciliary body, iris, iridocorneal angle and posterior lens capsule. (B) Sense ISH localization from various regions of eye for a test animal (high dose group, 1@4.5×1011 vg/eye) is shown. The localization of pattern of sense ISH signal is similar that of antisense ISH. Sense ISH signal is abundant in macula and in multifocal areas of central retina, particularly within ganglion cells and inner nuclear layer cells. ISH signal are more uniformly detected in peripheral retina. Mild to moderate levels of ISH signal are present in ciliary body, iris, iridocorneal angle and posterior lens capsule.

FIG. 11 provides semiquantitative scoring of (A) antisense ISH signal and (B) sense ISH signal in various regions of cynomolgus eyes. A modified H-score was developed considering number of ISH signal per cell and percent cells expressing ISH signal as described in the methods section. Note that ISH signal is higher with high dose group compared to low dose group, but there was no significant differences between interim and terminal sacrifice animal in the same dose group.

FIG. 12 illustrates antisense signal in some GRM6+ bipolar cells by dual labeling ISH experiments. Dual antisense and GRM6 ISH labeling experiments in eye section of a test animal. Single plex experiments show GRM6 (a marker of ON bipolar cells) ISH signal (signal in panel A) specifically in outer stripes of inner nuclear layer and antisense signal (signal in Panel B) in nerve fiber layer, ganglion cells, inner plexiform layer and inner nuclear cells. As shown in panel C (circles), few cells show dual labeling of antisense and GRM6 ISH signal.

FIG. 13 illustrates exemplary vector transduction in retina by AAV capsid protein immunostaining. AAV capsid immunostaining was noted nuclei of ganglion cells and inner nuclear layers of peripheral retina suggesting exemplary vector transduction. Representative images from section of a test animal shown. Presence of capsid proteins in lens capsule and inner limiting membrane suggests vector adherence to these membranes.

FIG. 14 illustrates opsin protein immunostaining in retina of exemplary vector injected cynomolgus macaques. Representative images from eye sections of a test animal with anti-red/green (a.k.a. long/medium wave) opsin immunostain is shown. Immunostaining with anti-red/green demonstrate MW-opsin was noted in in central (macula) and peripheral retina and in ciliary body. Inset shows punctate immunostaining in ganglion cells. Immunostaining in peripheral retina spans the entire thickness of neuroretina in multifocal areas, resembling a Muller cell pattern. Ciliary body and nonpigmented epithelium show minimal staining, but there was no immunostaining in optic nerve or other regions of eye.

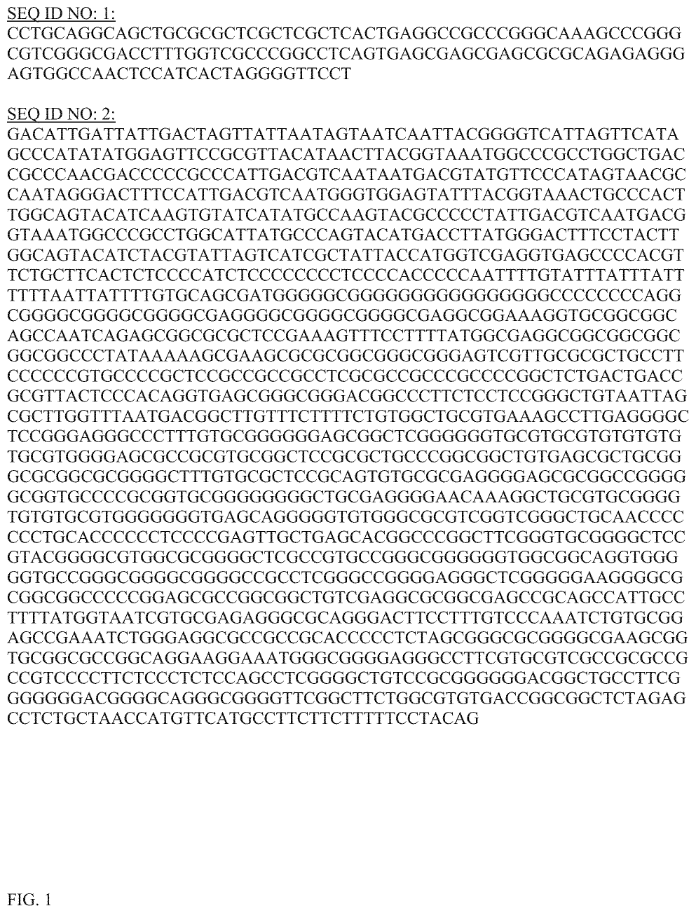

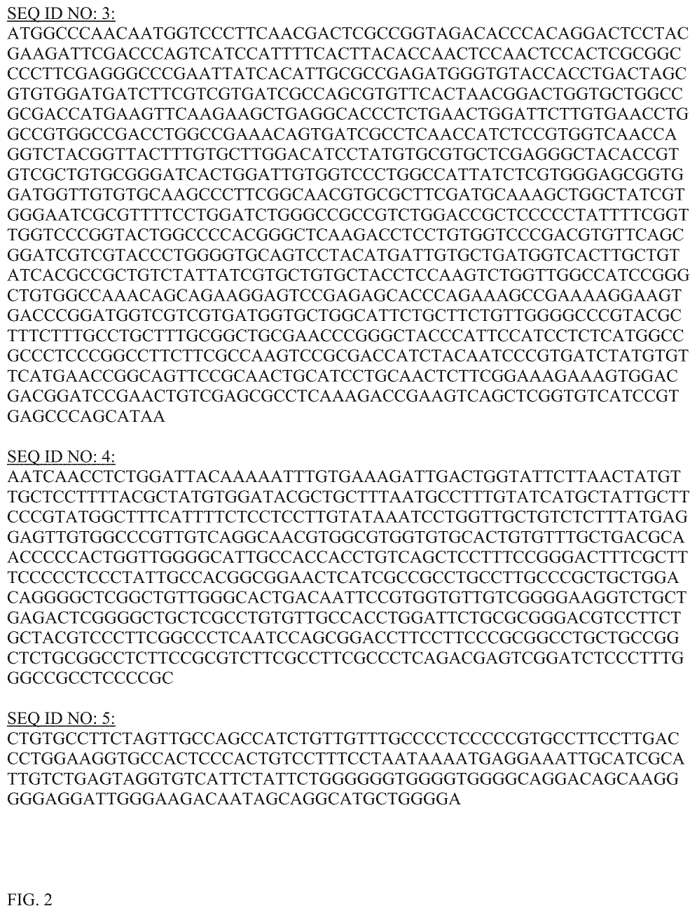

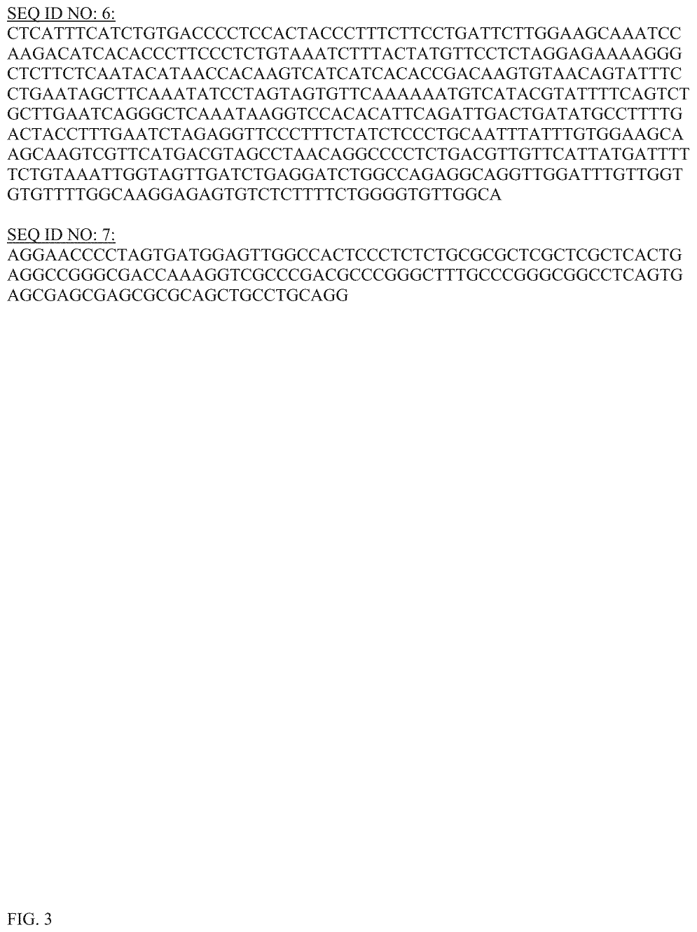

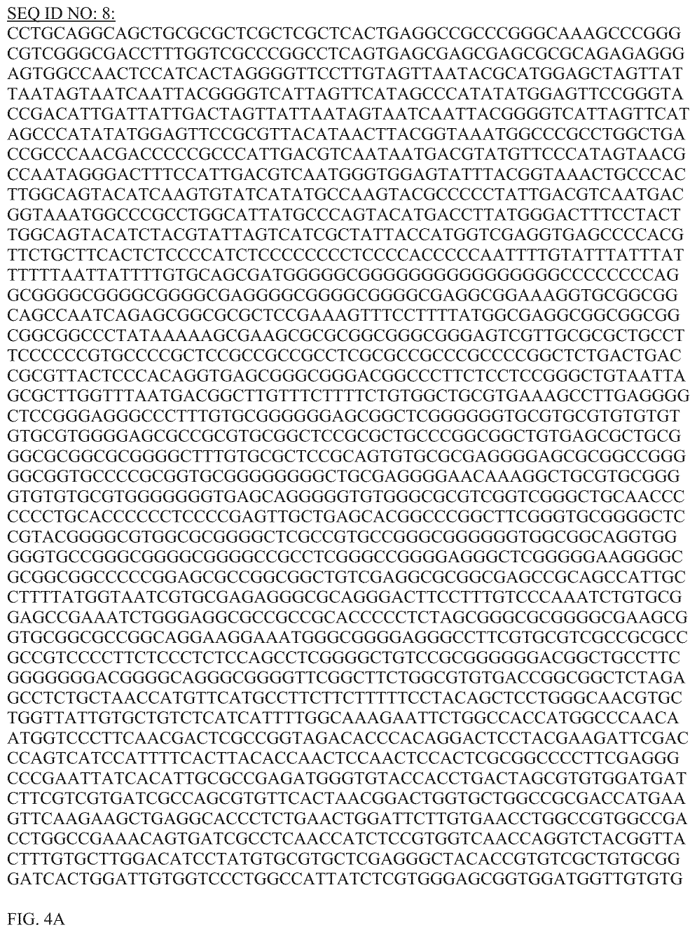

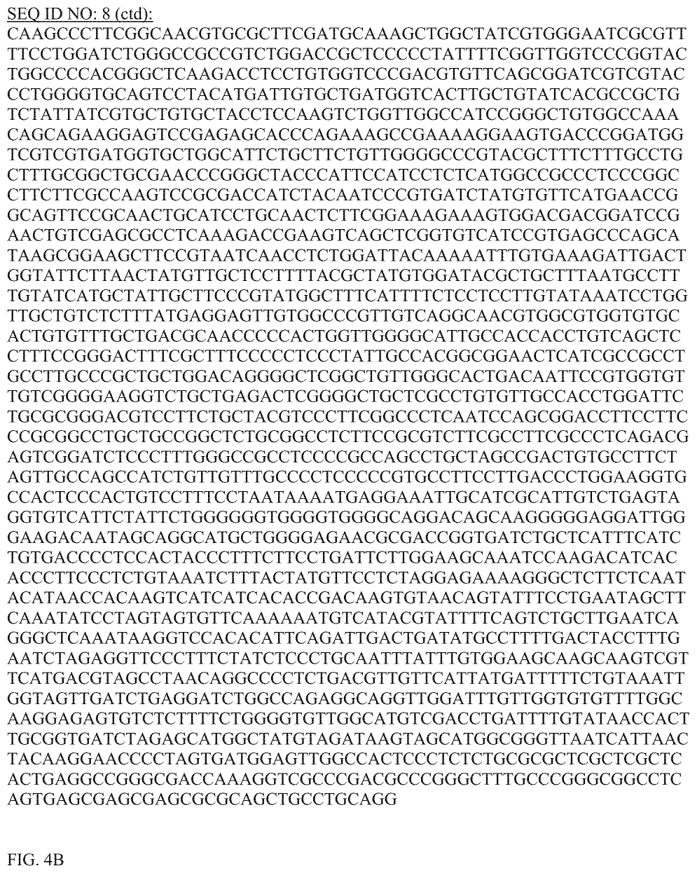

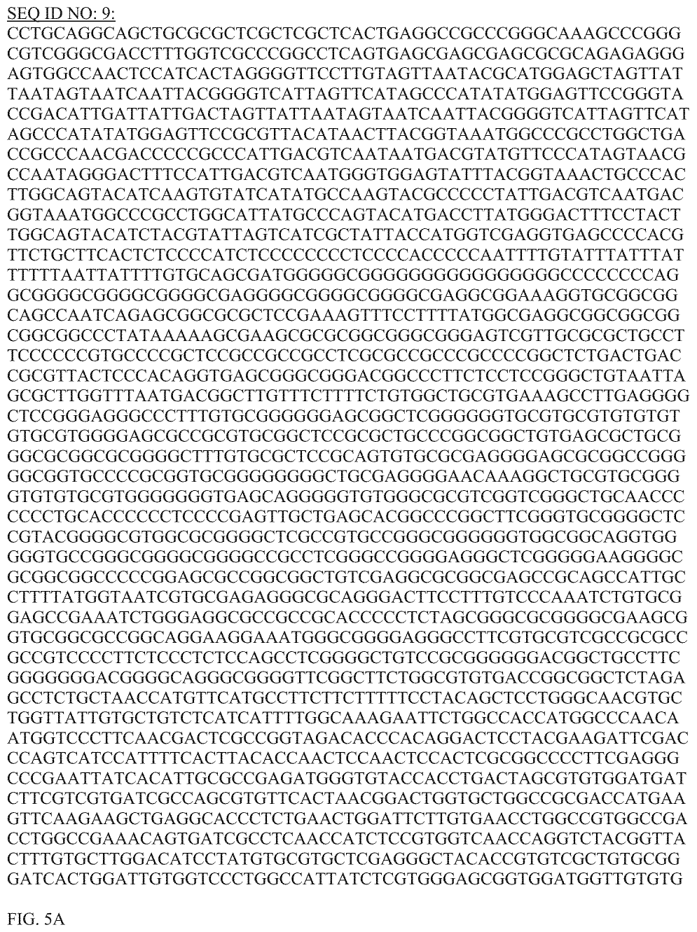

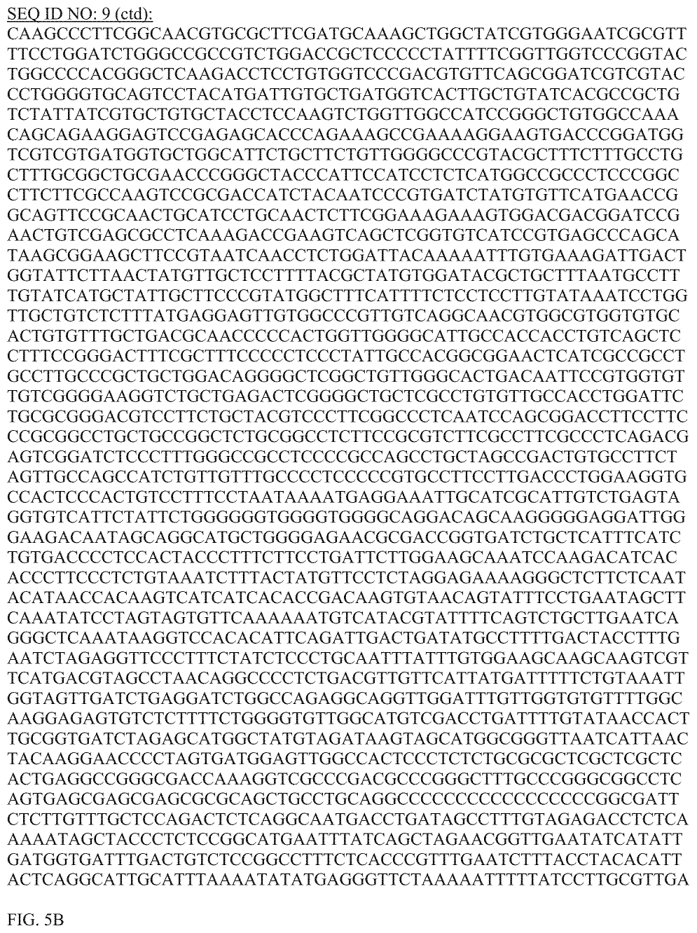

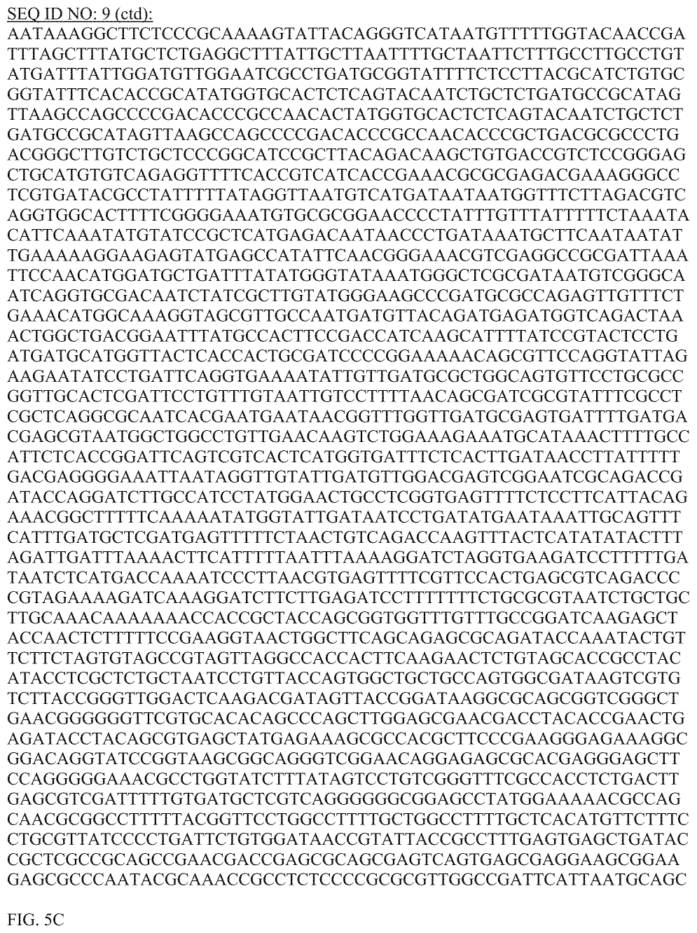

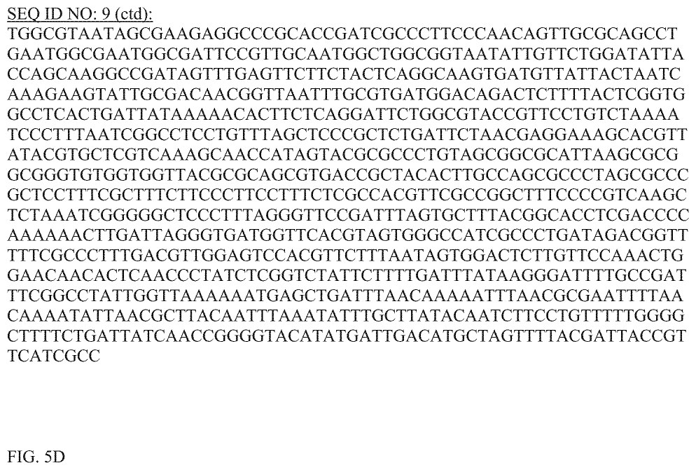

### DETAILED DESCRIPTION

The present disclosure relates to pharmaceutical compositions, methods of treatment of eye diseases or conditions comprising administering a gene therapy, a vector, or a construct by intraocular, intravitreal or subretinal injection into an eye of a primate (e.g., a monkey or a human) comprising a polynucleotide sequence (e.g., cDNA) that encodes MW-opsin. Upon intraocular, intravitreal or subretinal injection of a gene therapy, a vector, or a construct, comprising a polynucleotide sequence that encodes MW-opsin, the MW-opsin transgene is expressed in vivo in target cells or tissue to generate MW-opsin protein or gene product to produce a therapeutic effect.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein. The terminology of the present disclosure is for the purpose of describing particular cases only and is not intended to be limiting of companions, methods and compositions of this disclosure.

The compositions and methods of this disclosure as described herein may employ, unless otherwise indicated, conventional techniques and descriptions of molecular biology (including recombinant techniques), cell biology, biochemistry, immunochemistry and ophthalmic techniques, which are within the skill of those who practice in the art. Such conventional techniques include methods for observing and analyzing the retina, or vision in a subject, cloning and propagation of recombinant virus, formulation of a pharmaceutical composition, and biochemical purification and immunochemistry. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual(2002) (all from Cold Spring Harbor Laboratory Press); Stryer. L., Biochemistry (4th Ed.) W.H. Freeman, N.Y. (1995); Gait, “Oligonucleotide Synthesis: A Practical Approach” IRL Press, London (1984); Nelson and Cox, Lehninger, Principles of Biochemistry, 3rd Ed., W.H. Freeman Pub., New York (2000); and Berg et al., Biochemistry, 5th Ed., W.H. Freeman Pub., New York (2002), all of which are herein incorporated by reference in their entirety for all purposes.

The section headings used herein are for organizational purposes only and are not to be construed as limiting the subject matter described.

### Definitions

Unless otherwise defined, all technical terms used herein have the same meaning as commonly understood by one of ordinary skill in the art.

The terminology used herein is for the purpose of describing particular cases only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The term “comprising” as used herein is synonymous with “including” or “containing,” and is inclusive or open-ended.

Any reference to “or” herein is intended to encompass “and/or” unless otherwise stated. As used herein, the term “about” a number refers to that number plus or minus 10% of that number. The term “about” a range refers to that range minus 10% of its lowest value and plus 10% of its greatest value.

The term “subject,” “patient,” or “individual” refers to primates, including non-human primates, e.g., African green monkeys and rhesus monkeys, and humans. In preferred embodiments, the subject is a human or a human patient.

The terms “treat,” “treating,” “treatment,” “ameliorate,” or “ameliorating” and other grammatical equivalents as used herein, include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disease being treated. Also, a therapeutic benefit is achieved with the eradication or amelioration of one or more of the physiological symptoms associated with the underlying disease such that an improvement is observed in the patient, notwithstanding that, in some embodiments, the patient is still afflicted with the underlying disease. In certain aspects, for prophylactic benefit, the pharmaceutical compositions are administered to a patient at risk of developing a particular disease, or to a patient reporting one or more of the physiological symptoms of a disease, even if a diagnosis of the disease has not been made.

The terms “administer,” “administering,” “administration,” and the like, as used herein, can refer to the methods that are used to enable delivery of therapeutic or pharmaceutical compositions to the desired site of biological action. These methods include intraocular, intravitreal, or subretinal injection to an eye.

The terms “effective amount,” “therapeutically effective amount,” or “pharmaceutically effective amount” as used herein, can refer to a sufficient amount of at least one pharmaceutical composition or compound being administered which will relieve to some extent one or more of the symptoms of the disease or condition being treated.

The term “pharmaceutically acceptable” as used herein, can refer to a material, such as a carrier or diluent, which does not abrogate the biological activity or properties of a compound disclosed herein, and is relatively nontoxic (i.e., when the material is administered to an individual it does not cause undesirable biological effects nor does it interact in a deleterious manner with any of the components of the composition in which it is contained).

The term “pharmaceutical composition” or simply “composition” as used herein, can refer to a biologically active compound, optionally mixed with at least one pharmaceutically acceptable chemical component, such as, though not limited to carriers, stabilizers, diluents, dispersing agents, suspending agents, thickening agents, excipients and the like.

An “AAV vector” or “rAAV vector” as used herein refers to an adeno-associated virus (AAV) vector or a recombinant AAV (rAAV) vector comprising a polynucleotide sequence not of AAV origin (i.e., a polynucleotide heterologous to AAV such as a polynucleotide sequence that encodes a therapeutic transgene, e.g., MW-opsin), typically a sequence of interest for the genetic transformation of a cell. In general, the heterologous polynucleotide is flanked by at least one, and generally by two, AAV inverted terminal repeat sequences (ITRs). The term rAAV vector encompasses both rAAV vector particles and rAAV vector plasmids. A rAAV vector may either be single-stranded (ssAAV) or self-complementary (scAAV).

An “AAV virus” or “AAV viral particle” or “rAAV vector particle” refers to a viral particle composed of at least one AAV capsid protein (typically by all of the capsid proteins of a wild-type AAV) and a polynucleotide rAAV vector. If the particle comprises a heterologous polynucleotide (i.e. a polynucleotide other than a wild-type AAV genome such as a transgene to be delivered to a mammalian cell), it is typically referred to as an “rAAV vector particle” or simply an “rAAV vector.” Thus, production of rAAV particle necessarily includes production of rAAV vector, as such a vector is contained within a rAAV particle.

The term “packaging” as used herein can refer to a series of intracellular events that can result in the assembly and encapsidation of a rAAV particle. AAV “rep” and “cap” genes refer to polynucleotide sequences encoding replication and encapsidation proteins of adeno-associated virus. AAV rep and cap are referred to herein as AAV “packaging genes.”

The term “polypeptide” can encompass both naturally-occurring and non-naturally occurring proteins (e.g., a fusion protein), peptides, fragments, mutants, derivatives and analogs thereof. A polypeptide may be monomeric, dimeric, trimeric, or polymeric. Further, a polypeptide may comprise a number of different domains each of which has one or more distinct activities. For the avoidance of doubt, a “polypeptide” may be any length greater two amino acids.

As used herein, “polypeptide variant” refers to a polypeptide whose sequence contains an amino acid modification. In some instances, the modification can be an insertion, duplication, deletion, rearrangement or substitution of one or more amino acids compared to the amino acid sequence of a reference protein or polypeptide, such as a native or wild-type protein. A variant may have one or more amino acid point substitutions, in which a single amino acid at a position has been changed to another amino acid, one or more insertions and/or deletions, in which one or more amino acids are inserted or deleted, respectively, in the sequence of the reference protein, and/or truncations of the amino acid sequence at either or both the amino or carboxy termini. A variant can have the same or a different biological activity compared to the reference protein, or the unmodified protein.

In some embodiments, a variant can have, for example, at least 80%, 81%, 82%, 83%, 84%, 85%, 86%, 87%, 88%, 89%, 90%, 91%, 92%, 93%, 94%, 95%, 96%, 97%, 98%, 99% or 100% overall sequence homology to its counterpart reference protein, wherein the reference protein can be naturally occurring or non-naturally occurring, or a derivative or variant of a naturally occurring protein. In some embodiments, a variant can have at least about 90% overall sequence homology to the wild-type protein. In some embodiments, a variant exhibits at least about 95%, at least about 98%, at least about 99%, at least about 99.5%, or at least about 99.9% overall sequence identity.

The term “polynucleotide” or “nucleic acid” refers to messenger RNA (mRNA), RNA, genomic RNA (gRNA), plus strand RNA (RNA(+)), minus strand RNA (RNA(−)), genomic DNA (gDNA), complementary DNA (cDNA) or recombinant DNA. Polynucleotides include single and double stranded polynucleotides. Preferably, polynucleotides include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. In various illustrative embodiments, the present invention contemplates, in part, polynucleotides comprising expression vectors, viral vectors, and transfer plasmids, and compositions, and cells comprising the same.

In particular embodiments, polynucleotides are provided by this invention that encode at least about 5, 10, 25, 50, 100, 150, 200, 250, 300, 350, 400, 500, 1000, 1250, 1500, 1750, or 2000 or more contiguous amino acid residues of a polypeptide, as well as all intermediate lengths. It will be readily understood that “intermediate lengths,” in this context, means any length between the quoted values, such as 6, 7, 8, 9, etc.; 101, 102, 103, etc.; 151, 152, 153, etc.; 201, 202, 203, etc.

As used herein, “polynucleotide variant” refers to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations inclusive of mutations, additions, deletions, and substitutions can be made to a reference polynucleotide whereby the altered polynucleotide retains the biological function or activity of the reference polynucleotide.

As used herein, “recombinant” can refer to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such polynucleotides. Thus, for example, a protein synthesized by a microorganism is recombinant, for example, if it is synthesized from an mRNA synthesized from a recombinant gene present in the cell.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity.” A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the premessenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

Moreover, it will be appreciated by those of ordinary skill in the art that, as a result of the degeneracy of the genetic code, there are many nucleotide sequences that encode a polypeptide, or fragment of variant thereof, as described herein. Some of these polynucleotides bear minimal homology to the nucleotide sequence of any native gene. Nonetheless, polynucleotides that vary due to differences in codon usage are specifically contemplated by the present invention, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

“Operatively linked” or “operably linked” or “coupled” can refer to a juxtaposition of genetic elements, wherein the elements are in a relationship permitting them to operate in an expected manner. For instance, a promoter can be operatively linked to a coding region if the promoter helps initiate transcription of the coding sequence. There may be intervening residues between the promoter and coding region so long as this functional relationship is maintained.

The term “expression vector” or “expression construct” or “cassette” or “plasmid” or simply “vector” can include any type of genetic construct, including AAV or rAAV vectors, containing a polynucleotide coding for a gene product in which part or all of the polynucleotide encoding sequence is capable of being transcribed and is adapted for gene therapy. The transcript can be translated into a protein. In some cases, it may be partially translated or not translated. In certain aspects, expression includes both transcription of a gene and translation of mRNA into a gene product. In other aspects, expression only includes transcription of the polynucleotide encoding genes of interest. An expression vector can also comprise control elements operatively linked to the encoding region to facilitate expression of the protein in target cells. The combination of control elements and a gene or genes to which they are operably linked for expression can sometimes be referred to as an “expression cassette,” a large number of which are known and available in the art or can be readily constructed from components that are available in the art.

The “control elements” or “regulatory sequences” present in an expression vector are those non-translated regions of the vector—origin of replication, selection cassettes, promoters, enhancers, introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

The term “promoter” as used herein refers to a recognition site of a polynucleotide (DNA or RNA) to which an RNA polymerase binds. An RNA polymerase initiates and transcribes polynucleotides operably linked to the promoter.

The term “enhancer” refers to a segment of DNA, which contains sequences capable of providing enhanced transcription and in some instances can function independent of their orientation relative to another control sequence. An enhancer can function cooperatively or additively with promoters and/or other enhancer elements.

The term “heterologous” can refer to an entity that is genotypically distinct from that of the rest of the entity to which it is being compared. For example, a polynucleotide introduced by genetic engineering techniques into a plasmid or vector derived from a different species can be a heterologous polynucleotide. A promoter removed from its native coding sequence and operatively linked to a coding sequence with which it is not naturally found linked can be a heterologous promoter.

The term “MW-opsin” or “medium wavelength opsin” or “medium wavelength cone opsin” refers to Homo sapiens cone opsin 1, i.e., medium-wave sensitive OPN1MW (NCBI RefSeq Accession No. NM_000513, Version NM_000513.2), functional fragments or functional derivatives thereof, and fusion proteins comprising the same. MW-opsin has a λₘₐₓ of ˜530 nm in the green region of the electromagnetic spectrum. It is also referred to as the “green opsin”, “M opsin” or “MWS opsin.”

Ranges: throughout this disclosure, various aspects can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range. All specified ranges also include the endpoints unless otherwise stated.

### Vectors

Various viral vectors can be used in gene therapy, including adenovirus, adeno-associated virus, lentivirus, herpes simplex virus, or a retrovirus.

In some embodiments, pharmaceutical compositions and methods of the disclosure provide for delivery of a polynucleotide sequence (e.g., cDNA sequence) encoding a MW-opsin transgene to retinal cells in a human subject or patient in need thereof (e.g., a patient diagnosed with age-related macular degeneration (AMD), retinitis pigmentosa). Delivery of the polynucleotide of a therapeutic transgene to a patient using a delivery system, such as rAAV or a viral vector, is also referred to as gene therapy.

In some embodiments, delivery of a MW-opsin encoding polynucleotide sequence can be performed using any suitable “vector” (also referred to as “gene delivery” or “gene transfer vehicle”). Vector (e.g., rAAV), delivery vehicle, gene delivery vehicle or gene transfer vehicle, can encompass any suitable macromolecule or complex of molecules comprising a polynucleotide to be delivered to a target cell, e.g., retinal cells, including photoreceptor, a retinal ganglion cell, a Muller cell, a bipolar cell, an amacrine cell, a horizontal cell, or a retinal pigmented epithelium cell. In some cases, a target cell can be any cell to which the polynucleotide molecule or gene is delivered.

The composition and methods of the disclosure provide for any suitable method for delivery of a MW-opsin transgene polynucleotide sequence into an eye or retinal cells of a non-human primate or human subject. In a specific embodiment, the MW-opsin transgene has the polynucleotide sequence of SEQ ID NO: 3. SEQ ID NO: 3 has the sequence of ATGGCCCAACAATGGTCCCTTCAACGACTCGCCGGTAGACACCCACAGGACTCCTAC GAAGATTCGACCCAGTCATCCATTTTCACTTACACCAACTCCAACTCCACTCGCGGC CCCTTCGAGGGCCCGAATTATCACATTGCGCCGAGATGGGTGTACCACCTGACTAGC GTGTGGATGATCTTCGTCGTGATCGCCAGCGTGTTCACTAACGGACTGGTGCTGGCC GCGACCATGAAGTTCAAGAAGCTGAGGCACCCTCTGAACTGGATTCTTGTGAACCTG GCCGTGGCCGACCTGGCCGAAACAGTGATCGCCTCAACCATCTCCGTGGTCAACCA GGTCTACGGTTACTTTGTGCTTGGACATCCTATGTGCGTGCTCGAGGGCTACACCGT GTCGCTGTGCGGGATCACTGGATTGTGGTCCCTGGCCATTATCTCGTGGGAGCGGTG GATGGTTGTGTGCAAGCCCTTCGGCAACGTGCGCTTCGATGCAAAGCTGGCTATCGT GGGAATCGCGTTTTCCTGGATCTGGGCCGCCGTCTGGACCGCTCCCCCTATTTTCGGT TGGTCCCGGTACTGGCCCCACGGGCTCAAGACCTCCTGTGGTCCCGACGTGTTCAGC GGATCGTCGTACCCTGGGGTGCAGTCCTACATGATTGTGCTGATGGTCACTTGCTGT ATCACGCCGCTGTCTATTATCGTGCTGTGCTACCTCCAAGTCTGGTTGGCCATCCGGG CTGTGGCCAAACAGCAGAAGGAGTCCGAGAGCACCCAGAAAGCCGAAAAGGAAGT GACCCGGATGGTCGTCGTGATGGTGCTGGCATTCTGCTTCTGTTGGGGCCCGTACGC TTTCTTTGCCTGCTTTGCGGCTGCGAACCCGGGCTACCCATTCCATCCTCTCATGGCC GCCCTCCCGGCCTTCTTCGCCAAGTCCGCGACCATCTACAATCCCGTGATCTATGTGT TCATGAACCGGCAGTTCCGCAACTGCATCCTGCAACTCTTCGGAAAGAAAGTGGAC GACGGATCCGAACTGTCGAGCGCCTCAAAGACCGAAGTCAGCTCGGTGTCATCCGT GAGCCCAGCATAA. SEQ ID NO: 3 is a codon-optimized polynucleotide sequence that increases expression of human MW-opsin protein, for example, relative to expression of human MW-opsin protein encoded by a polynucleotide sequence that is not codon-optimized. In a specific embodiment, the MW-opsin transgene has the polynucleotide sequence that is at least 85%, 90%, or 95% identical to SEQ ID NO: 3, which sequence is a codon-optimized polynucleotide sequence that increases expression of human MW-opsin protein, for example, relative to expression of human MW-opsin protein encoded by a polynucleotide sequence that is not codon-optimized. In some cases, delivery of the polynucleotide, or gene therapy is formulated or adapted for intravitreal injection into an eye of a non-human primate or human subject.

In some embodiments, suitable vectors include, but are not limited to, viral vectors such as adenoviruses, adeno-associated viruses (AAV), lentiviruses, herpes simplex viruses, and retroviruses, liposomes, lipid-containing complexes, nanoparticles, and other macromolecular complexes capable of delivery of a polynucleotide to retinal cells. In some embodiments, the viral vector comprises a eukaryotic promoter operably linked to the polynucleotide e.g., a cytomegalovirus (CMV) promoter or a constitutive promoter. In one embodiment, the promoter is a chicken 3-actin promoter. In a specific embodiment, the promoter has the polynucleotide sequence that is at least 85%, 90%, 95%, or 100% identical to SEQ ID NO: 2. SEQ ID NO: 2 has the sequence of

GACATTGATTATTGACTAGTTATTAATAGTAATCAATTACGGGGTCATT
AGTTCATAGCCCATATATGGAGTTCCGCGTTACATAACTTACGGTAAAT
GGCCCGCCTGGCTGACCGCCCAACGACCCCCGCCCATTGACGTCAATAA
TGACGTATGTTCCCATAGTAACGCCAATAGGGACTTTCCATTGACGTCA
ATGGGTGGAGTATTTACGGTAAACTGCCCACTTGGCAGTACATCAAGTG
TATCATATGCCAAGTACGCCCCCTATTGACGTCAATGACGGTAAATGGC
CCGCCTGGCATTATGCCCAGTACATGACCTTATGGGACTTTCCTACTTG
GCAGTACATCTACGTATTAGTCATCGCTATTACCATGGTCGAGGTGAGC
CCCACGTTCTGCTTCACTCTCCCCATCTCCCCCCCCTCCCCACCCCCAA
TTTTGTATTTATTTATTTTTTAATTATTTTGTGCAGCGATGGGGGCGGG
GGGGGGGGGGGGGCCCCCCCCAGGCGGGGCGGGGCGGGGCGAGGGGCGG
GGCGGGGCGAGGCGGAAAGGTGCGGCGGCAGCCAATCAGAGCGGCGCGC
TCCGAAAGTTTCCTTTTATGGCGAGGCGGCGGCGGCGGCGGCCCTATAA
AAAGCGAAGCGCGCGGCGGGCGGGAGTCGTTGCGCGCTGCCTTCCCCCC
GTGCCCCGCTCCGCCGCCGCCTCGCGCCGCCCGCCCCGGCTCTGACTGA
CCGCGTTACTCCCACAGGTGAGCGGGCGGGACGGCCCTTCTCCTCCGGG
CTGTAATTAGCGCTTGGTTTAATGACGGCTTGTTTCTTTTCTGTGGCTG
CGTGAAAGCCTTGAGGGGCTCCGGGAGGGCCCTTTGTGCGGGGGGAGCG
GCTCGGGGGGTGCGTGCGTGTGTGTGTGCGTGGGGAGCGCCGCGTGCGG
CTCCGCGCTGCCCGGCGGCTGTGAGCGCTGCGGGCGCGGCGCGGGGCTT
TGTGCGCTCCGCAGTGTGCGCGAGGGGAGCGCGGCCGGGGGCGGTGCCC
CGCGGTGCGGGGGGGGCTGCGAGGGGAACAAAGGCTGCGTGCGGGGTGT
GTGCGTGGGGGGGTGAGCAGGGGGTGTGGGCGCGTCGGTCGGGCTGCAA
CCCCCCCTGCACCCCCCTCCCCGAGTTGCTGAGCACGGCCCGGCTTCGG
GTGCGGGGCTCCGTACGGGGCGTGGCGCGGGGCTCGCCGTGCCGGGCGG
GGGGTGGCGGCAGGTGGGGGTGCCGGGCGGGGCGGGGCCGCCTCGGGCC
GGGGAGGGCTCGGGGGAAGGGGCGCGGCGGCCCCCGGAGCGCCGGCGGC
TGTCGAGGCGCGGCGAGCCGCAGCCATTGCCTTTTATGGTAATCGTGCG
AGAGGGCGCAGGGACTTCCTTTGTCCCAAATCTGTGCGGAGCCGAAATC
TGGGAGGCGCCGCCGCACCCCCTCTAGCGGGCGCGGGGCGAAGCGGTGC
GGCGCCGGCAGGAAGGAAATGGGCGGGGAGGGCCTTCGTGCGTCGCCGC
GCCGCCGTCCCCTTCTCCCTCTCCAGCCTCGGGGCTGTCCGCGGGGGGA
CGGCTGCCTTCGGGGGGGACGGGGCAGGGCGGGGTTCGGCTTCTGGCGT
GTGACCGGCGGCTCTAGAGCCTCTGCTAACCATGTTCATGCCTTCTTCT
TTTTCCTACAG.

In another embodiment, the promoter is a human synapsin hSyn promoter (Berry et al. (2019) Nat. Comm. 10:1221), a CAG promoter (Miyazaki et al. (1989) Gene 79:269); a glutamate metabotropic receptor-6 (grm6) promoter (Cronin et al. (2014) EMBO Mol. Med 6:1175); a Pleiades promoter (Portales-Casamar et al. (2010) Proc. Natl. Acad Sci. USA 107:16589); a choline acetyltransferase (ChAT) promoter (Misawa et al. (1992) Biol. Chem. 267:20392); a vesicular glutamate transporter (V-glut) promoter (Zhang et al. (2011) Brain Res. 1377:1); a glutamic acid decarboxylase (GAD) promoter (Rasmussen et al. (2007) Brain Res. 1144:19; Ritter et al. (2016) J. Gene Med 18:27); a cholecystokinin (CCK) promoter (Ritter et al. (2016) J. Gene Med 18:27); a parvalbumin (PV) promoter; a somatostatin (SST) promoter; a neuropeptide Y (NPY) promoter; and a vasoactive intestinal peptide (VIP) promoter. Suitable promoters include, but are not limited to, a red cone opsin promoter, rhodopsin promoter, a rhodopsin kinase promoter, and a GluR promoter (e.g., a GluR6 promoter). Suitable promoters include, but are not limited to, a vitelliform macular dystrophy 2 (VMD2) gene promoter, and an interphotoreceptor retinoid-binding protein (IRBP) gene promoter. Also suitable for use is an L7 promoter (Oberdick et al. (1990) Science 248:223), a thy-1 promoter, a recoverin promoter (Wiechmann and Howard (2003) Curr. Eye Res. 26:25); the BEST promoter; ProB4 promoter (Juttner, J. et al., (2019) Nat. Neurosci. 22:1345-1356); ProC2 promoter; ProA18 promoter; ProB2 promoter; ProA6 promoter; ProA7 promoter; ProA1 promoter; ProA4 promoter; ProC22 promoter; ProD3 promoter; ProD4 promoter; ProD5 promoter; ProD6 promoter; ProA14 promoter; ProA36 promoter; ProD1 promoter; ProA5 promoter; ProB1 promoter; ProA27 promoter; ProC29 promoter; ProB15 promoter; ProA9 promoter; ProC8 promoter; ProA21 promoter; SNCG promoter; ProC17 promoter; SynP156 promoter; ProA18 promoter; ProB4 promoter; ProB12 promoter; and a calbindin promoter. In particular embodiments, promoters operative in mammalian cells comprise an AT-rich region located approximately 25 to 30 bases upstream from the site where transcription is initiated and/or another sequence found 70 to 80 bases upstream from the start of transcription.

In some embodiments, RNA can include a transcript of a gene ofinterest (e.g., MW-opsin), introns, untranslated regions (UTRs), termination sequences and the like. In other embodiments, DNA can include, but are not limited to, sequences such as promoter sequences, a gene of interest (e.g., MW-opsin), UTRs, enhancer sequences, intron sequences, termination sequences, ITR sequences, and the like. In a specific embodiment, the enhancer has the polynucleotide sequence of SEQ ID NO: 4. SEQ ID NO: 4 has the sequence of AATCAACCTCTGGATTACAAAAATTTGTGAAAGATTGACTGGTATTCTTAACTATGT TGCTCCTTTTACGCTATGTGGATACGCTGCTTTAATGCCTTTGTATCATGCTATTGCTT CCCGTATGGCTTTCATTTTCTCCTCCTTGTATAAATCCTGGTTGCTGTCTCTTTATGAG GAGTTGTGGCCCGTTGTCAGGCAACGTGGCGTGGTGTGCACTGTGTTTGCTGACGCA ACCCCCACTGGTTGGGGCATTGCCACCACCTGTCAGCTCCTTTCCGGGACTTTCGCTT TCCCCCTCCCTATTGCCACGGCGGAACTCATCGCCGCCTGCCTTGCCCGCTGCTGGA CAGGGGCTCGGCTGTTGGGCACTGACAATTCCGTGGTGTTGTCGGGGAAGGTCTGCT GAGACTCGGGGCTGCTCGCCTGTGTTGCCACCTGGATTCTGCGCGGGACGTCCTTCT GCTACGTCCCTTCGGCCCTCAATCCAGCGGACCTTCCTTCCCGCGGCCTGCTGCCGG CTCTGCGGCCTCTTCCGCGTCTTCGCCTTCGCCCTCAGACGAGTCGGATCTCCCTTTG GGCCGCCTCCCCGC. SEQ ID NO: 4 was synthesized to be regulatory compliant and is a variant of the wild-type WPR enhancer.

In a specific embodiment, the intron has the polynucleotide sequence of SEQ ID NO: 6. SEQ ID NO: 6 has the sequence of CTCATTTCATCTGTGACCCCTCCACTACCCTTTCTTCCTGATTCTTGGAAGCAAATCC AAGACATCACACCCTTCCCTCTGTAAATCTTTACTATGTTCCTCTAGGAGAAAAGGG CTCTTCTCAATACATAACCACAAGTCATCATCACACCGACAAGTGTAACAGTATTTC CTGAATAGCTTCAAATATCCTAGTAGTGTTCAAAAAATGTCATACGTATTTTCAGTCT GCTTGAATCAGGGCTCAAATAAGGTCCACACATTCAGATTGACTGATATGCCTTTTG ACTACCTTTGAATCTAGAGGTTCCCTTTCTATCTCCCTGCAATTTATTTGTGGAAGCA AGCAAGTCGTTCATGACGTAGCCTAACAGGCCCCTCTGACGTTGTTCATTATGATTTT TCTGTAAATTGGTAGTTGATCTGAGGATCTGGCCAGAGGCAGGTTGGATTTGTTGGT GTGTTTTGGCAAGGAGAGTGTCTCTTTTCTGGGGTGTTGGCA. SEQ ID NO: 6 was synthesized and incorporated into the recombinant expression vector to keep the transgene close to the packing size limit of 4.5-4.7 kB. In addition, the inclusion of SEQ ID NO: 6 in the recombinant expression vector reduces risk of non-sense/shuffled DNA that would give rise to cryptic splice sites and alternative coding regions. SEQ ID NO: 6 was designed to minimize GC content. Other intron polynucleotide sequences contemplated for use in the invention include any of the human SW-opsin, MW-opsin, and LW-opsin intron polynucleotide sequences shortened to keep the transgene close to the packing size limit of 4.5-4.7 kB, and/or substituted with the appropriate number and type of nucleotide residues to minimize GC content.

In a specific embodiment, the termination sequence has the polynucleotide sequence of SEQ ID NO: 5. SEQ ID NO: 5 has the sequence of

CTGTGCCTTCTAGTTGCCAGCCATCTGTTGTTTGCCCCTCCCCCG
TGCCTTCCTTGACCCTGGAAGGTGCCACTCCCACTGTCCTTTCCT
AATAAAATGAGGAAATTGCATCGCATTGTCTGAGTAGGTGTCATT
CTATTCTGGGGGGTGGGGTGGGGCAGGACAGCAAGGGGGAGGATT
GGGAAGACAATAGCAGGCATGCTGGGGA.

In a specific embodiment, the 5′ ITR has the polynucleotide sequence of SEQ ID NO: 1. SEQ ID NO: 1 has the sequence of CCTGCAGGCAGCTGCGCGCTCGCTCGCTCACTGAGGCCGCCCGGGCAAAGCCCGGG CGTCGGGCGACCTTTGGTCGCCCGGCCTCAGTGAGCGAGCGAGCGCGCAGAGAGGG AGTGGCCAACTCCATCACTAGGGGTTCCT. In another specific embodiment, the 3′ ITR has the polynucleotide sequence of SEQ ID NO: 7. SEQ ID NO: 7 has the sequence of AGGAACCCCTAGTGATGGAGTTGGCCACTCCCTCTCTGCGCGCTCGCTCGCTCACTG AGGCCGGGCGACCAAAGGTCGCCCGACGCCCGGGCTTTGCCCGGGCGGCCTCAGTG AGCGAGCGAGCGCGCAGCTGCCTGCAGG. SEQ ID NOs: 1 and 7 were designed to ensure the stability of the recombinant expression vector over several rounds of propagation/amplification.

In some embodiments, the rAAV and/or plasmid used to generate rAAV viruses comprises the following polynucleotide elements: a first ITR sequence; a promoter sequence; a sequence encoding MW-opsin; an enhancer sequence; a polyA/termination sequence, an intron sequence; and a second ITR sequence. In some embodiments, a linker sequence is used between each of these polynucleotide elements. In some embodiments, the polynucleotide elements are present in the rAAV and/or plasmid in the following 5′ to 3′ orientation: a first ITR sequence; a promoter sequence; a sequence encoding MW-opsin; an enhancer sequence; a polyA/termination sequence, an intron sequence; and a second ITR sequence. In further embodiments, the rAAV and/or plasmid comprises the polynucleotide sequence of SEQ ID NO: 8. SEQ ID NO: 8 has the sequence of

CCTGCAGGCAGCTGCGCGCTCGCTCGCTCACTGAGGCCGCCCGGGCA
AAGCCCGGGCGTCGGGCGACCTTTGGTCGCCCGGCCTCAGTGAGCGA
GCGAGCGCGCAGAGAGGGAGTGGCCAACTCCATCACTAGGGGTTCCT
TGTAGTTAATACGCATGGAGCTAGTTATTAATAGTAATCAATTACGG
GGTCATTAGTTCATAGCCCATATATGGAGTTCCGGGTACCGACATTG
ATTATTGACTAGTTATTAATAGTAATCAATTACGGGGTCATTAGTTC
ATAGCCCATATATGGAGTTCCGCGTTACATAACTTACGGTAAATGGC
CCGCCTGGCTGACCGCCCAACGACCCCCGCCCATTGACGTCAATAAT
GACGTATGTTCCCATAGTAACGCCAATAGGGACTTTCCATTGACGTC
AATGGGTGGAGTATTTACGGTAAACTGCCCACTTGGCAGTACATCAA
GTGTATCATATGCCAAGTACGCCCCCTATTGACGTCAATGACGGTAA
ATGGCCCGCCTGGCATTATGCCCAGTACATGACCTTATGGGACTTTC
CTACTTGGCAGTACATCTACGTATTAGTCATCGCTATTACCATGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGGGCCCCCCCCAGGCGGGGCGGGG
CGGGGCGAGGGGCGGGGCGGGGCGAGGCGGAAAGGTGCGGCGGCAGC
CAATCAGAGCGGCGCGCTCCGAAAGTTTCCTTTTATGGCGAGGCGGC
GGCGGCGGCGGCCCTATAAAAAGCGAAGCGCGCGGCGGGCGGGAGTC
GTTGCGCGCTGCCTTCCCCCCGTGCCCCGCTCCGCCGCCGCCTCGCG
CCGCCCGCCCCGGCTCTGACTGACCGCGTTACTCCCACAGGTGAGCG
GGCGGGACGGCCCTTCTCCTCCGGGCTGTAATTAGCGCTTGGTTTAA
TGACGGCTTGTTTCTTTTCTGTGGCTGCGTGAAAGCCTTGAGGGGCT
CCGGGAGGGCCCTTTGTGCGGGGGGAGCGGCTCGGGGGGTGCGTGCG
TGTGTGTGTGCGTGGGGAGCGCCGCGTGCGGCTCCGCGCTGCCCGGC
GGCTGTGAGCGCTGCGGGCGCGGCGCGGGGCTTTGTGCGCTCCGCAG
TGTGCGCGAGGGGAGCGCGGCCGGGGGCGGTGCCCCGCGGTGCGGGG
GGGGCTGCGAGGGGAACAAAGGCTGCGTGCGGGGTGTGTGCGTGGGG
GGGTGAGCAGGGGGTGTGGGCGCGTCGGTCGGGCTGCAACCCCCCCT
GCACCCCCCTCCCCGAGTTGCTGAGCACGGCCCGGCTTCGGGTGCGG
GGCTCCGTACGGGGCGTGGCGCGGGGCTCGCCGTGCCGGGCGGGGGG
TGGCGGCAGGTGGGGGTGCCGGGCGGGGCGGGGCCGCCTCGGGCCGG
GGAGGGCTCGGGGGAAGGGGCGCGGCGGCCCCCGGAGCGCCGGCGGC
TGTCGAGGCGCGGCGAGCCGCAGCCATTGCCTTTTATGGTAATCGTG
CGAGAGGGCGCAGGGACTTCCTTTGTCCCAAATCTGTGCGGAGCCGA
AATCTGGGAGGCGCCGCCGCACCCCCTCTAGCGGGCGCGGGGCGAAG
CGGTGCGGCGCCGGCAGGAAGGAAATGGGCGGGGAGGGCCTTCGTGC
GTCGCCGCGCCGCCGTCCCCTTCTCCCTCTCCAGCCTCGGGGCTGTC
CGCGGGGGGACGGCTGCCTTCGGGGGGGACGGGGCAGGGCGGGGTTC
GGCTTCTGGCGTGTGACCGGCGGCTCTAGAGCCTCTGCTAACCATGT
TCATGCCTTCTTCTTTTTCCTACAGCTCCTGGGCAACGTGCTGGTTA
TTGTGCTGTCTCATCATTTTGGCAAAGAATTCTGGCCACCATGGCCC
AACAATGGTCCCTTCAACGACTCGCCGGTAGACACCCACAGGACTCC
TACGAAGATTCGACCCAGTCATCCATTTTCACTTACACCAACTCCAA
CTCCACTCGCGGCCCCTTCGAGGGCCCGAATTATCACATTGCGCCGA
GATGGGTGTACCACCTGACTAGCGTGTGGATGATCTTCGTCGTGATC
GCCAGCGTGTTCACTAACGGACTGGTGCTGGCCGCGACCATGAAGTT
CAAGAAGCTGAGGCACCCTCTGAACTGGATTCTTGTGAACCTGGCCG
TGGCCGACCTGGCCGAAACAGTGATCGCCTCAACCATCTCCGTGGTC
AACCAGGTCTACGGTTACTTTGTGCTTGGACATCCTATGTGCGTGCT
CGAGGGCTACACCGTGTCGCTGTGCGGGATCACTGGATTGTGGTCCC
TGGCCATTATCTCGTGGGAGCGGTGGATGGTTGTGTGCAAGCCCTTC
GGCAACGTGCGCTTCGATGCAAAGCTGGCTATCGTGGGAATCGCGTT
TTCCTGGATCTGGGCCGCCGTCTGGACCGCTCCCCCTATTTTCGGTT
GGTCCCGGTACTGGCCCCACGGGCTCAAGACCTCCTGTGGTCCCGAC
GTGTTCAGCGGATCGTCGTACCCTGGGGTGCAGTCCTACATGATTGT
GCTGATGGTCACTTGCTGTATCACGCCGCTGTCTATTATCGTGCTGT
GCTACCTCCAAGTCTGGTTGGCCATCCGGGCTGTGGCCAAACAGCAG
AAGGAGTCCGAGAGCACCCAGAAAGCCGAAAAGGAAGTGACCCGGAT
GGTCGTCGTGATGGTGCTGGCATTCTGCTTCTGTTGGGGCCCGTACG
CTTTCTTTGCCTGCTTTGCGGCTGCGAACCCGGGCTACCCATTCCAT
CCTCTCATGGCCGCCCTCCCGGCCTTCTTCGCCAAGTCCGCGACCAT
CTACAATCCCGTGATCTATGTGTTCATGAACCGGCAGTTCCGCAACT
GCATCCTGCAACTCTTCGGAAAGAAAGTGGACGACGGATCCGAACTG
TCGAGCGCCTCAAAGACCGAAGTCAGCTCGGTGTCATCCGTGAGCCC
AGCATAAGCGGAAGCTTCCGTAATCAACCTCTGGATTACAAAAATTT
GTGAAAGATTGACTGGTATTCTTAACTATGTTGCTCCTTTTACGCTA
TGTGGATACGCTGCTTTAATGCCTTTGTATCATGCTATTGCTTCCCG
TATGGCTTTCATTTTCTCCTCCTTGTATAAATCCTGGTTGCTGTCTC
TTTATGAGGAGTTGTGGCCCGTTGTCAGGCAACGTGGCGTGGTGTGC
ACTGTGTTTGCTGACGCAACCCCCACTGGTTGGGGCATTGCCACCAC
CTGTCAGCTCCTTTCCGGGACTTTCGCTTTCCCCCTCCCTATTGCCA
CGGCGGAACTCATCGCCGCCTGCCTTGCCCGCTGCTGGACAGGGGCT
CGGCTGTTGGGCACTGACAATTCCGTGGTGTTGTCGGGGAAGGTCTG
CTGAGACTCGGGGCTGCTCGCCTGTGTTGCCACCTGGATTCTGCGCG
GGACGTCCTTCTGCTACGTCCCTTCGGCCCTCAATCCAGCGGACCTT
CCTTCCCGCGGCCTGCTGCCGGCTCTGCGGCCTCTTCCGCGTCTTCG
CCTTCGCCCTCAGACGAGTCGGATCTCCCTTTGGGCCGCCTCCCCGC
CAGCCTGCTAGCCGACTGTGCCTTCTAGTTGCCAGCCATCTGTTGTT
TGCCCCTCCCCCGTGCCTTCCTTGACCCTGGAAGGTGCCACTCCCAC
TGTCCTTTCCTAATAAAATGAGGAAATTGCATCGCATTGTCTGAGTA
GGTGTCATTCTATTCTGGGGGGTGGGGTGGGGCAGGACAGCAAGGGG
GAGGATTGGGAAGACAATAGCAGGCATGCTGGGGAGAACGCGACCGG
TGATCTGCTCATTTCATCTGTGACCCCTCCACTACCCTTTCTTCCTG
ATTCTTGGAAGCAAATCCAAGACATCACACCCTTCCCTCTGTAAATC
TTTACTATGTTCCTCTAGGAGAAAAGGGCTCTTCTCAATACATAACC
ACAAGTCATCATCACACCGACAAGTGTAACAGTATTTCCTGAATAGC
TTCAAATATCCTAGTAGTGTTCAAAAAATGTCATACGTATTTTCAGT
CTGCTTGAATCAGGGCTCAAATAAGGTCCACACATTCAGATTGACTG
ATATGCCTTTTGACTACCTTTGAATCTAGAGGTTCCCTTTCTATCTC
CCTGCAATTTATTTGTGGAAGCAAGCAAGTCGTTCATGACGTAGCCT
AACAGGCCCCTCTGACGTTGTTCATTATGATTTTTCTGTAAATTGGT
AGTTGATCTGAGGATCTGGCCAGAGGCAGGTTGGATTTGTTGGTGTG
TTTTGGCAAGGAGAGTGTCTCTTTTCTGGGGTGTTGGCATGTCGACC
TGATTTTGTATAACCACTTGCGGTGATCTAGAGCATGGCTATGTAGA
TAAGTAGCATGGCGGGTTAATCATTAACTACAAGGAACCCCTAGTGA
TGGAGTTGGCCACTCCCTCTCTGCGCGCTCGCTCGCTCACTGAGGCC
GGGCGACCAAAGGTCGCCCGACGCCCGGGCTTTGCCCGGGCGGCCTC
AGTGAGCGAGCGAGCGCGCAGCTGCCTGCAGG.

In further embodiments, the rAAV and/or plasmid comprises the polynucleotide sequence of SEQ ID NO: 9. SEQ ID NO: 9 has the sequence of

CCTGCAGGCAGCTGCGCGCTCGCTCGCTCACTGAGGCCGCCCGGGCA
AAGCCCGGGCGTCGGGCGACCTTTGGTCGCCCGGCCTCAGTGAGCGA
GCGAGCGCGCAGAGAGGGAGTGGCCAACTCCATCACTAGGGGTTCCT
TGTAGTTAATACGCATGGAGCTAGTTATTAATAGTAATCAATTACGG
GGTCATTAGTTCATAGCCCATATATGGAGTTCCGGGTACCGACATTG
ATTATTGACTAGTTATTAATAGTAATCAATTACGGGGTCATTAGTTC
ATAGCCCATATATGGAGTTCCGCGTTACATAACTTACGGTAAATGGC
CCGCCTGGCTGACCGCCCAACGACCCCCGCCCATTGACGTCAATAAT
GACGTATGTTCCCATAGTAACGCCAATAGGGACTTTCCATTGACGTC
AATGGGTGGAGTATTTACGGTAAACTGCCCACTTGGCAGTACATCAA
GTGTATCATATGCCAAGTACGCCCCCTATTGACGTCAATGACGGTAA
ATGGCCCGCCTGGCATTATGCCCAGTACATGACCTTATGGGACTTTC
CTACTTGGCAGTACATCTACGTATTAGTCATCGCTATTACCATGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGGGCCCCCCCCAGGCGGGGCGGGG
CGGGGCGAGGGGCGGGGCGGGGCGAGGCGGAAAGGTGCGGCGGCAGC
CAATCAGAGCGGCGCGCTCCGAAAGTTTCCTTTTATGGCGAGGCGGC
GGCGGCGGCGGCCCTATAAAAAGCGAAGCGCGCGGCGGGCGGGAGTC
GTTGCGCGCTGCCTTCCCCCCGTGCCCCGCTCCGCCGCCGCCTCGCG
CCGCCCGCCCCGGCTCTGACTGACCGCGTTACTCCCACAGGTGAGCG
GGCGGGACGGCCCTTCTCCTCCGGGCTGTAATTAGCGCTTGGTTTAA
TGACGGCTTGTTTCTTTTCTGTGGCTGCGTGAAAGCCTTGAGGGGCT
CCGGGAGGGCCCTTTGTGCGGGGGGAGCGGCTCGGGGGGTGCGTGCG
TGTGTGTGTGCGTGGGGAGCGCCGCGTGCGGCTCCGCGCTGCCCGGC
GGCTGTGAGCGCTGCGGGCGCGGCGCGGGGCTTTGTGCGCTCCGCAG
TGTGCGCGAGGGGAGCGCGGCCGGGGGCGGTGCCCCGCGGTGCGGGG
GGGGCTGCGAGGGGAACAAAGGCTGCGTGCGGGGTGTGTGCGTGGGG
GGGTGAGCAGGGGGTGTGGGCGCGTCGGTCGGGCTGCAACCCCCCCT
GCACCCCCCTCCCCGAGTTGCTGAGCACGGCCCGGCTTCGGGTGCGG
GGCTCCGTACGGGGCGTGGCGCGGGGCTCGCCGTGCCGGGCGGGGGG
TGGCGGCAGGTGGGGGTGCCGGGCGGGGCGGGGCCGCCTCGGGCCGG
GGAGGGCTCGGGGGAAGGGGCGCGGCGGCCCCCGGAGCGCCGGCGGC
TGTCGAGGCGCGGCGAGCCGCAGCCATTGCCTTTTATGGTAATCGTG
CGAGAGGGCGCAGGGACTTCCTTTGTCCCAAATCTGTGCGGAGCCGA
AATCTGGGAGGCGCCGCCGCACCCCCTCTAGCGGGCGCGGGGCGAAG
CGGTGCGGCGCCGGCAGGAAGGAAATGGGCGGGGAGGGCCTTCGTGC
GTCGCCGCGCCGCCGTCCCCTTCTCCCTCTCCAGCCTCGGGGCTGTC
CGCGGGGGGACGGCTGCCTTCGGGGGGGACGGGGCAGGGCGGGGTTC
GGCTTCTGGCGTGTGACCGGCGGCTCTAGAGCCTCTGCTAACCATGT
TCATGCCTTCTTCTTTTTCCTACAGCTCCTGGGCAACGTGCTGGTTA
TTGTGCTGTCTCATCATTTTGGCAAAGAATTCTGGCCACCATGGCCC
AACAATGGTCCCTTCAACGACTCGCCGGTAGACACCCACAGGACTCC
TACGAAGATTCGACCCAGTCATCCATTTTCACTTACACCAACTCCAA
CTCCACTCGCGGCCCCTTCGAGGGCCCGAATTATCACATTGCGCCGA
GATGGGTGTACCACCTGACTAGCGTGTGGATGATCTTCGTCGTGATC
GCCAGCGTGTTCACTAACGGACTGGTGCTGGCCGCGACCATGAAGTT
CAAGAAGCTGAGGCACCCTCTGAACTGGATTCTTGTGAACCTGGCCG
TGGCCGACCTGGCCGAAACAGTGATCGCCTCAACCATCTCCGTGGTC
AACCAGGTCTACGGTTACTTTGTGCTTGGACATCCTATGTGCGTGCT
CGAGGGCTACACCGTGTCGCTGTGCGGGATCACTGGATTGTGGTCCC
TGGCCATTATCTCGTGGGAGCGGTGGATGGTTGTGTGCAAGCCCTTC
GGCAACGTGCGCTTCGATGCAAAGCTGGCTATCGTGGGAATCGCGTT
TTCCTGGATCTGGGCCGCCGTCTGGACCGCTCCCCCTATTTTCGGTT
GGTCCCGGTACTGGCCCCACGGGCTCAAGACCTCCTGTGGTCCCGAC
GTGTTCAGCGGATCGTCGTACCCTGGGGTGCAGTCCTACATGATTGT
GCTGATGGTCACTTGCTGTATCACGCCGCTGTCTATTATCGTGCTGT
GCTACCTCCAAGTCTGGTTGGCCATCCGGGCTGTGGCCAAACAGCAG
AAGGAGTCCGAGAGCACCCAGAAAGCCGAAAAGGAAGTGACCCGGAT
GGTCGTCGTGATGGTGCTGGCATTCTGCTTCTGTTGGGGCCCGTACG
CTTTCTTTGCCTGCTTTGCGGCTGCGAACCCGGGCTACCCATTCCAT
CCTCTCATGGCCGCCCTCCCGGCCTTCTTCGCCAAGTCCGCGACCAT
CTACAATCCCGTGATCTATGTGTTCATGAACCGGCAGTTCCGCAACT
GCATCCTGCAACTCTTCGGAAAGAAAGTGGACGACGGATCCGAACTG
TCGAGCGCCTCAAAGACCGAAGTCAGCTCGGTGTCATCCGTGAGCCC
AGCATAAGCGGAAGCTTCCGTAATCAACCTCTGGATTACAAAAATTT
GTGAAAGATTGACTGGTATTCTTAACTATGTTGCTCCTTTTACGCTA
TGTGGATACGCTGCTTTAATGCCTTTGTATCATGCTATTGCTTCCCG
TATGGCTTTCATTTTCTCCTCCTTGTATAAATCCTGGTTGCTGTCTC
TTTATGAGGAGTTGTGGCCCGTTGTCAGGCAACGTGGCGTGGTGTGC
ACTGTGTTTGCTGACGCAACCCCCACTGGTTGGGGCATTGCCACCAC
CTGTCAGCTCCTTTCCGGGACTTTCGCTTTCCCCCTCCCTATTGCCA
CGGCGGAACTCATCGCCGCCTGCCTTGCCCGCTGCTGGACAGGGGCT
CGGCTGTTGGGCACTGACAATTCCGTGGTGTTGTCGGGGAAGGTCTG
CTGAGACTCGGGGCTGCTCGCCTGTGTTGCCACCTGGATTCTGCGCG
GGACGTCCTTCTGCTACGTCCCTTCGGCCCTCAATCCAGCGGACCTT
CCTTCCCGCGGCCTGCTGCCGGCTCTGCGGCCTCTTCCGCGTCTTCG
CCTTCGCCCTCAGACGAGTCGGATCTCCCTTTGGGCCGCCTCCCCGC
CAGCCTGCTAGCCGACTGTGCCTTCTAGTTGCCAGCCATCTGTTGTT
TGCCCCTCCCCCGTGCCTTCCTTGACCCTGGAAGGTGCCACTCCCAC
TGTCCTTTCCTAATAAAATGAGGAAATTGCATCGCATTGTCTGAGTA
GGTGTCATTCTATTCTGGGGGGTGGGGTGGGGCAGGACAGCAAGGGG
GAGGATTGGGAAGACAATAGCAGGCATGCTGGGGAGAACGCGACCGG
TGATCTGCTCATTTCATCTGTGACCCCTCCACTACCCTTTCTTCCTG
ATTCTTGGAAGCAAATCCAAGACATCACACCCTTCCCTCTGTAAATC
TTTACTATGTTCCTCTAGGAGAAAAGGGCTCTTCTCAATACATAACC
ACAAGTCATCATCACACCGACAAGTGTAACAGTATTTCCTGAATAGC
TTCAAATATCCTAGTAGTGTTCAAAAAATGTCATACGTATTTTCAGT
CTGCTTGAATCAGGGCTCAAATAAGGTCCACACATTCAGATTGACTG
ATATGCCTTTTGACTACCTTTGAATCTAGAGGTTCCCTTTCTATCTC
CCTGCAATTTATTTGTGGAAGCAAGCAAGTCGTTCATGACGTAGCCT
AACAGGCCCCTCTGACGTTGTTCATTATGATTTTTCTGTAAATTGGT
AGTTGATCTGAGGATCTGGCCAGAGGCAGGTTGGATTTGTTGGTGTG
TTTTGGCAAGGAGAGTGTCTCTTTTCTGGGGTGTTGGCATGTCGACC
TGATTTTGTATAACCACTTGCGGTGATCTAGAGCATGGCTATGTAGA
TAAGTAGCATGGCGGGTTAATCATTAACTACAAGGAACCCCTAGTGA
TGGAGTTGGCCACTCCCTCTCTGCGCGCTCGCTCGCTCACTGAGGCC
GGGCGACCAAAGGTCGCCCGACGCCCGGGCTTTGCCCGGGCGGCCTC
AGTGAGCGAGCGAGCGCGCAGCTGCCTGCAGGCCCCCCCCCCCCCCC
CCGGCGATTCTCTTGTTTGCTCCAGACTCTCAGGCAATGACCTGATA
GCCTTTGTAGAGACCTCTCAAAAATAGCTACCCTCTCCGGCATGAAT
TTATCAGCTAGAACGGTTGAATATCATATTGATGGTGATTTGACTGT
CTCCGGCCTTTCTCACCCGTTTGAATCTTTACCTACACATTACTCAG
GCATTGCATTTAAAATATATGAGGGTTCTAAAAATTTTTATCCTTGC
GTTGAAATAAAGGCTTCTCCCGCAAAAGTATTACAGGGTCATAATGT
TTTTGGTACAACCGATTTAGCTTTATGCTCTGAGGCTTTATTGCTTA
ATTTTGCTAATTCTTTGCCTTGCCTGTATGATTTATTGGATGTTGGA
ATCGCCTGATGCGGTATTTTCTCCTTACGCATCTGTGCGGTATTTCA
CACCGCATATGGTGCACTCTCAGTACAATCTGCTCTGATGCCGCATA
GTTAAGCCAGCCCCGACACCCGCCAACACTATGGTGCACTCTCAGTA
CAATCTGCTCTGATGCCGCATAGTTAAGCCAGCCCCGACACCCGCCA
ACACCCGCTGACGCGCCCTGACGGGCTTGTCTGCTCCCGGCATCCGC
TTACAGACAAGCTGTGACCGTCTCCGGGAGCTGCATGTGTCAGAGGT
TTTCACCGTCATCACCGAAACGCGCGAGACGAAAGGGCCTCGTGATA
CGCCTATTTTTATAGGTTAATGTCATGATAATAATGGTTTCTTAGAC
GTCAGGTGGCACTTTTCGGGGAAATGTGCGCGGAACCCCTATTTGTT
TATTTTTCTAAATACATTCAAATATGTATCCGCTCATGAGACAATAA
CCCTGATAAATGCTTCAATAATATTGAAAAAGGAAGAGTATGAGCCA
TATTCAACGGGAAACGTCGAGGCCGCGATTAAATTCCAACATGGATG
CTGATTTATATGGGTATAAATGGGCTCGCGATAATGTCGGGCAATCA
GGTGCGACAATCTATCGCTTGTATGGGAAGCCCGATGCGCCAGAGTT
GTTTCTGAAACATGGCAAAGGTAGCGTTGCCAATGATGTTACAGATG
AGATGGTCAGACTAAACTGGCTGACGGAATTTATGCCACTTCCGACC
ATCAAGCATTTTATCCGTACTCCTGATGATGCATGGTTACTCACCAC
TGCGATCCCCGGAAAAACAGCGTTCCAGGTATTAGAAGAATATCCTG
ATTCAGGTGAAAATATTGTTGATGCGCTGGCAGTGTTCCTGCGCCGG
TTGCACTCGATTCCTGTTTGTAATTGTCCTTTTAACAGCGATCGCGT
ATTTCGCCTCGCTCAGGCGCAATCACGAATGAATAACGGTTTGGTTG
ATGCGAGTGATTTTGATGACGAGCGTAATGGCTGGCCTGTTGAACAA
GTCTGGAAAGAAATGCATAAACTTTTGCCATTCTCACCGGATTCAGT
CGTCACTCATGGTGATTTCTCACTTGATAACCTTATTTTTGACGAGG
GGAAATTAATAGGTTGTATTGATGTTGGACGAGTCGGAATCGCAGAC
CGATACCAGGATCTTGCCATCCTATGGAACTGCCTCGGTGAGTTTTC
TCCTTCATTACAGAAACGGCTTTTTCAAAAATATGGTATTGATAATC
CTGATATGAATAAATTGCAGTTTCATTTGATGCTCGATGAGTTTTTC
TAACTGTCAGACCAAGTTTACTCATATATACTTTAGATTGATTTAAA
ACTTCATTTTTAATTTAAAAGGATCTAGGTGAAGATCCTTTTTGATA
ATCTCATGACCAAAATCCCTTAACGTGAGTTTTCGTTCCACTGAGCG
TCAGACCCCGTAGAAAAGATCAAAGGATCTTCTTGAGATCCTTTTTT
TCTGCGCGTAATCTGCTGCTTGCAAACAAAAAAACCACCGCTACCAG
ACCAACTCTTTTTCCGAAGGTAACTGGCTTCAGCAGAGCGCAGATAC
CACGGTGGTTTGTTTGCCGGATCAAGAGCTAATACTGTTCTTCTAGT
GTAGCCGTAGTTAGGCCACCACTTCAAGAACTCTGTAGCACCGCCTA
CATACCTCGCTCTGCTAATCCTGTTACCAGTGGCTGCTGCCAGTGGC
GATAAGTCGTGTCTTACCGGGTTGGACTCAAGACGATAGTTACCGGA
TAAGGCGCAGCGGTCGGGCTGAACGGGGGGTTCGTGCACACAGCCCA
GCTTGGAGCGAACGACCTACACCGAACTGAGATACCTACAGCGTGAG
CTATGAGAAAGCGCCACGCTTCCCGAAGGGAGAAAGGCGGACAGGTA
TCCGGTAAGCGGCAGGGTCGGAACAGGAGAGCGCACGAGGGAGCTTC
CAGGGGGAAACGCCTGGTATCTTTATAGTCCTGTCGGGTTTCGCCAC
CTCTGACTTGAGCGTCGATTTTTGTGATGCTCGTCAGGGGGGCGGAG
CCTATGGAAAAACGCCAGCAACGCGGCCTTTTTACGGTTCCTGGCCT
TTTGCTGGCCTTTTGCTCACATGTTCTTTCCTGCGTTATCCCCTGAT
TCTGTGGATAACCGTATTACCGCCTTTGAGTGAGCTGATACCGCTCG
CCGCAGCCGAACGACCGAGCGCAGCGAGTCAGTGAGCGAGGAAGCGG
AAGAGCGCCCAATACGCAAACCGCCTCTCCCCGCGCGTTGGCCGATT
CATTAATGCAGCTGGCGTAATAGCGAAGAGGCCCGCACCGATCGCCC
TTCCCAACAGTTGCGCAGCCTGAATGGCGAATGGCGATTCCGTTGCA
ATGGCTGGCGGTAATATTGTTCTGGATATTACCAGCAAGGCCGATAG
TTTGAGTTCTTCTACTCAGGCAAGTGATGTTATTACTAATCAAAGAA
GTATTGCGACAACGGTTAATTTGCGTGATGGACAGACTCTTTTACTC
GGTGGCCTCACTGATTATAAAAACACTTCTCAGGATTCTGGCGTACC
GTTCCTGTCTAAAATCCCTTTAATCGGCCTCCTGTTTAGCTCCCGCT
CTGATTCTAACGAGGAAAGCACGTTATACGTGCTCGTCAAAGCAACC
ATAGTACGCGCCCTGTAGCGGCGCATTAAGCGCGGCGGGTGTGGTGG
TTACGCGCAGCGTGACCGCTACACTTGCCAGCGCCCTAGCGCCCGCT
CCTTTCGCTTTCTTCCCTTCCTTTCTCGCCACGTTCGCCGGCTTTCC
CCGTCAAGCTCTAAATCGGGGGCTCCCTTTAGGGTTCCGATTTAGTG
CTTTACGGCACCTCGACCCCAAAAAACTTGATTAGGGTGATGGTTCA
CGTAGTGGGCCATCGCCCTGATAGACGGTTTTTCGCCCTTTGACGTT
GGAGTCCACGTTCTTTAATAGTGGACTCTTGTTCCAAACTGGAACAA
CACTCAACCCTATCTCGGTCTATTCTTTTGATTTATAAGGGATTTTG
CCGATTTCGGCCTATTGGTTAAAAAATGAGCTGATTTAACAAAAATT
TAACGCGAATTTTAACAAAATATTAACGCTTACAATTTAAATATTTG
CTTATACAATCTTCCTGTTTTTGGGGCTTTTCTGATTATCAACCGGG
GTACATATGATTGACATGCTAGTTTTACGATTACCGTTCATCGCC.

In some embodiments, the recombinant expression vector comprises a first inverted terminal repeat (ITR) polynucleotide sequence, a promoter polynucleotide sequence operably linked to a polynucleotide sequence encoding a medium-wavelength cone opsin (MW-opsin) transgene, an enhancer polynucleotide sequence, a polyA polynucleotide sequence, an intron polynucleotide sequence, and a second ITR polynucleotide sequence. In some embodiments, the recombinant expression vector further comprises a polynucleotide that confers resistance to an antibiotic. In yet further embodiments, that antibiotic is ampicillin and/or kanamycin.

In some embodiments, the present disclosure provides a recombinant virus, such as recombinant adeno-associated virus (rAAV) as a vector for delivery and expression of MW-opsin, or any functional fragment or variant thereof, in a subject.

In some embodiments, any suitable viral vectors can be engineered or optimized for use with the compositions and methods of the disclosure. For example, recombinant viral vectors derived from adenovirus (Ad) or adeno-associated virus (AAV) can be altered such that it is replication-defective in human or primate subjects. In some embodiments, hybrid viral vector systems can be obtained using methods known to one skilled in the art and used to deliver a polynucleotide encoding MW-opsin to retinal cells. In some embodiments, a viral delivery system or gene therapy can integrate a polynucleotide sequence comprising a MW-opsin transgene into the target cell genome (e.g., genome of retinal cells) and result in stable gene expression of the gene over time. In some embodiments, the MW-opsin transgene is not integrated into the target cell genome, and is expressed from a plasmid or vector introduced into the targeted cells.

In one embodiment, the recombinant expression vector is a recombinant viral vector. In specific embodiments, the recombinant viral vector is an adeno-associated viral vector, a lentiviral vector, a herpes simplex vector, or a retroviral vector. In some embodiments, a suitable viral vector for delivering a polynucleotide sequence of MW-opsin to retinal cells is AAV or rAAV, which are small non-enveloped single-stranded DNA viruses. rAAV are non-pathogenic human parvoviruses and can be made to be dependent on helper viruses, including adenovirus, herpes simplex virus, vaccinia virus and CMV, for replication. Exposure to wild-type (wt) AAV is not associated or known to cause any human pathologies and is common in the general population, making AAV or rAAV a suitable delivery system for gene therapy. AAV and rAAV used for gene therapy for delivery of a therapeutic transgene, e.g., MW-opsin, can be of any serotype. In some embodiments, pharmaceutical compositions and methods of the disclosure provide for use of any suitable AAV serotype, including AAV1, AAV2, AAV2.5, AAV3, AAV4, AAV5, AAV6, AAV7, AAV8, AAV9, AAV10, AAV 11, AAV12, rh10, AAV-DJ, and any hybrid or chimeric AAV thereof. In some embodiments, the serotype used is based on tropism of the virus, or infectivity of a target cell of interest. In some embodiments, AAV2 or rAAV2 is used to deliver a polynucleotide sequence encoding MW-opsin into an eye or retinal cells of a subject via intraocular, intravitreal, or subretinal injection.

In some embodiments, AAV or rAAV viruses, particles, or virions comprising a variant capsid protein having increased infectivity of target cells, e.g. retinal cells, are used to increase transduction of retinal cells or to increase targeting of gene delivery to retinal cells in a subject. In some embodiments, the rAAV virion comprises an amino acid modification in a capsid protein GH loop/loop IV of the AAV capsid protein. In some cases, the site of modification is a solvent-accessible portion of the GH loop/loop IV of the AAV capsid protein. In some embodiments, a rAAV virion comprises a variant AAV capsid protein that comprises an insertion of from 5 amino acids to 11 amino acids, e.g., 7 amino acid sequence, in the GH loop of a capsid protein relative to a corresponding parental AAV capsid protein, and wherein the variant capsid protein confers increased infectivity of a retinal cell compared to the infectivity of the retinal cell by an AAV virion comprising the corresponding parental or unmodified AAV capsid protein. Several AAV capsid variants are known, including the 7m8 variant. Other AAV capsid variants are disclosed in WO 2018/022905, herein incorporated by reference in their entirety. For example, AAV capsid variants contemplated for use in the invention include those have protein sequences of (1) LAKDATKNA (SEQ ID NO:10); (2) PAHQDTTKNA (SEQ ID NO: 11); (3) LAHQDTTKNA (SEQ ID NO:12); (4) LATTSQNKPA (SEQ ID NO:13); (5) LAISDQTKHA (SEQ ID NO:14); (6) IARGVAPSSA (SEQ ID NO:15); (7) LAPDSTTRSA (SEQ ID NO:16); (8) LAKGTELKPA (SEQ ID NO:17); (9) LAIIDATKNA (SEQ ID NO:18); (10) LAVDGAQRSA (SEQ ID NO:19); (11) PAPQDTTKKA (SEQ ID NO:20); (12) LPHQDTTKNA (SEQ ID NO:21); (13) LAKDATKTIA (SEQ ID NO:22); (14) LAKQQSASTA (SEQ ID NO:23); (15) LAKSDQSKPA (SEQ ID NO:24); (16) LSHQDTTKNA (SEQ ID NO:25); (17) LAANQPSKPA (SEQ ID NO:26); (18) LAVSDSTKAA (SEQ ID NO:27); (19) LAAQGTAKKPA (SEQ ID NO:28); (20) LAPDQTTRNA (SEQ ID NO:29); (21) LAASDSTKAA (SEQ ID NO:30); (22) LAPQDTTKNA (SEQ ID NO:31); (23) LAKADETRPA (SEQ ID NO:32); (24) LAHQDTAKNA (SEQ ID NO:33); (25) LAHQDTKKNA (SEQ ID NO:34); (26) LAHQDTTKHA (SEQ ID NO:35); (27) LAHQDTTKKA (SEQ ID NO:36); (28) LAHQDTTRNA (SEQ ID NO:37); (29) LAHQDTTNA (SEQ ID NO:38); (30) LAHQGTTKNA (SEQ ID NO:39); (31) LAHQVTTKNA (SEQ ID NO:40); (32) LAISDQSKPA (SEQ ID NO:41); (33) LADATKTA (SEQ ID NO:42); (34) LAKDTTKNA (SEQ ID NO:43); (35) LAKSDQSRPA (SEQ ID NO:44); (36) LAPQDTKKNA (SEQ ID NO:45); (37) LATSDSTKAA (SEQ ID NO:46); (38) LAVDGSQRSA (SEQ ID NO:47); (39) LPISDQTKHA (SEQ ID NO:48); (40) LPKDATKTIA (SEQ ID NO:49); (41) LPPQDTTKNA (SEQ ID NO:50); (42) PAPQDTTKNA (SEQ ID NO:51); (43) QAHQDTTKNA (SEQ ID NO:52); (44) LAHETSPRPA (SEQ ID NO:53); (45) LAKSTSTAPA (SEQ ID NO:54); (46) LADQDTTKNA (SEQ ID NO:55); (47) LAESDQSKPA (SEQ ID NO:56); (48) LAHKDTTKNA (SEQ ID NO:57); (49) LAHKTQQKM (SEQ ID NO:58); (50) LAHQDTTENA (SEQ ID NO:59); (51) LAHQDTTINA (SEQ ID NO:60); (52) LAHQDTTKKT (SEQ ID NO:61); (53) LAHQDTTKND (SEQ ID NO:62); (54) LAHQDTTKNT (SEQ ID NO:63); (55) LAHQDTTKNV (SEQ ID NO: 64); (56) LAHQDTTK™ (SEQ ID NO: 65); (57) LAHQNTTKNA (SEQ ID NO: 66); (58) LAHRDTTKNA (SEQ ID NO: 67); (59) LAISDQTNHA (SEQ ID NO: 68); (60) LAKQKSASTA (SEQ ID NO: 69); (61) LAKSDQCKPA (SEQ ID NO: 70); (62) LAKSDQSKPD (SEQ ID NO: 71); (63) LAKSDQSNPA (SEQ ID NO: 72); (64) LAKSYQSKPA (SEQ ID NO: 73); (65) LANQDTTKNA (SEQ ID NO: 74); (66) LAPQNTTKNA (SEQ ID NO: 75); (67) LAPSSIQKPA (SEQ ID NO: 76); (68) LAQQDTTKNA (SEQ ID NO: 77); (69) LAYQDTTKNA (SEQ ID NO: 78); (70) LDHQDTTKNA (SEQ ID NO: 79); (71) LDHQDTTKSA (SEQ ID NO: 80); (72) LGHQDTTKNA (SEQ ID NO:81); (73) LPHQDTTKND (SEQ ID NO: 82); (74) LPHQDTTKNT (SEQ ID NO: 83); (75) LPHQDTTNNA (SEQ ID NO: 84); (76) LTHQDTTKNA (SEQ ID NO: 85); (77) LTKDATKTIA (SEQ ID NO: 86); (78) LTPQDTTKNA (SEQ ID NO:87); (79) LVHQDTTKNA (SEQ ID NO: 88). Other AAV capsid variants contemplated for use in the invention include those have protein sequences of (1) KDATKN (SEQ ID NO:89); (2) HQDTTKN (SEQ ID NO:90); (3) HQDTTKN (SEQ ID NO:91); (4) TTSQNKP (SEQ ID NO:92); (5) ISDQTKH (SEQ ID NO:93); (6) RGVAPSS (SEQ ID NO:94); (7) PDSTTRS (SEQ ID NO:95); (8) KGTELKP (SEQ ID NO:96); (9) IIDATKN (SEQ ID NO:97); (10) VDGAQRS (SEQ ID NO:98); (11) PQDTTKK (SEQ ID NO:99); (12) HQDTTKN (SEQ ID NO:100); (13) KDATKTI (SEQ ID NO:101); (14) KQQSAST (SEQ ID NO:102); (15) KSDQSKP (SEQ ID NO:103); (16) HQDTTKN (SEQ ID NO:104); (17) ANQPSKP (SEQ ID NO:105); (18) VSDSTKA (SEQ ID NO:106); (19) AQGTAKKP (SEQ ID NO:107); (20) PDQTTRN (SEQ ID NO:108); (21) ASDSTKA (SEQ ID NO:109); (22) PQDTTKN (SEQ ID NO:110); (23) KADETRP (SEQ ID NO:111); (24) HQDTAKN (SEQ ID NO:112); (25) HQDTKKN (SEQ ID NO:113); (26) HQDTTKH (SEQ ID NO:114); (27) HQDTTKK (SEQ ID NO:115); (28) HQDTTRN (SEQ ID NO:116); (29) HQDTTN (SEQ ID NO:117); (30) HQGTTKN (SEQ ID NO:118); (31) HQVTTKN (SEQ ID NO:119); (32) ISDQSKP (SEQ IDNO:120); (33) DATKT (SEQ IDNO:121); (34) KDTTKN (SEQ IDNO:122); (35) KSDQSRP (SEQ ID NO:123); (36) PQDTKKN (SEQ ID NO:124); (37) TSDSTKA (SEQ ID NO:125); (38) VDGSQRS (SEQ ID NO:126); (39) ISDQTKH (SEQ ID NO:127); (40) KDATKTI (SEQ ID NO:128); (41) PQDTTKN (SEQ ID NO:129); (42) PQDTTKN (SEQ ID NO:130); (43) HQDTTKN (SEQ ID NO:131); (44) HETSPRP (SEQ ID NO:132); (45) KSTSTAP (SEQ ID NO:133); (46) DQDTTKN (SEQ ID NO:134); (47) ESDQSKP (SEQ ID NO:135); (48) HKDTTKN (SEQ ID NO:136); (49) HKTQQK (SEQ ID NO:137); (50) HQDTTEN (SEQ ID NO:138); (51) HQDTTIN (SEQ ID NO:139); (52) HQDTTKK (SEQ ID NO:140); (53) HQDTTKN (SEQ ID NO:141); (54) HQDTTKN (SEQ ID NO:142); (55) HQDTTKN (SEQ ID NO: 143); (56) HQDTTKT (SEQ ID NO: 144); (57) HQNTTKN (SEQ ID NO: 145); (58) HRDTTKN (SEQ ID NO: 146); (59) ISDQTNH (SEQ ID NO: 147); (60) KQKSAST (SEQ ID NO: 148); (61) KSDQCKP (SEQ ID NO: 149); (62) KSDQSKP (SEQ ID NO: 150); (63) KSDQSNP (SEQ ID NO: 151); (64) KSYQSKP (SEQ ID NO: 152); (65) NQDTTKN (SEQ ID NO: 153); (66) PQNTTKN (SEQ ID NO: 154); (67) PSSIQKP (SEQ ID NO: 155); (68) QQDTTKN (SEQ ID NO: 156); (69) YQDTTKN (SEQ ID NO: 157); (70) HQDTTKN (SEQ ID NO: 158); (71) HQDTTKS (SEQ ID NO: 159); (72) HQDTTKN (SEQ ID NO:160); (81) HQDTTKN (SEQ ID NO: 161); (74) HQDTTKN (SEQ ID NO: 162); (75) HQDTTNN (SEQ ID NO: 163); (76) HQDTTKN (SEQ ID NO: 164); (77) KDATKTI (SEQ ID NO: 165); (78) PQDTTKN (SEQ ID NO: 166); and (79) HQDTTKN (SEQ ID NO: 167). Thus, in a further embodiment, the variant capsid polypeptide has a sequence selected from the group consisting of SEQ ID NOs: 10-167.

Yet other AAV capsid variants contemplated for use in the invention are disclosed in WO 2021/243085, herein incorporated by reference in their entirety. For example, AAV capsid variants contemplated for use in the invention include those have protein sequences of (1) HQDTTKN (SEQ ID NO:168); (2) LGETTRA (SEQ ID NO:169); (3) HQDTTRP (SEQ ID NO:170); (4) RQDTTKN (SEQ ID NO:171); (5) HQDSTKN (SEQ ID NO:172); (6) HQDATKN (SEQ ID NO:173); (7) HQDTKKP (SEQ ID NO:174); (8) LSETTRP (SEQ ID NO:175); (9) HQDTTKK (SEQ ID NO:176); (10) LGEATRP (SEQ ID NO:177); (11) LGETTRT (SEQ ID NO:178); (12) LSEATRP (SEQ ID NO:179); (13) KDETKNS (SEQ ID NO:180); (14) LGETTKP (SEQ ID NO:181); (15) HQATTKN (SEQ ID NO:182); (16) LAHQDTTKNS (SEQ ID NO:183); (17) LALGETTRAA (SEQ ID NO: 184); (18) LAHQDTTRPA (SEQ ID NO: 185); (19) LARQDTTKNA (SEQ ID NO: 186); (20) LAHQDSTKNA (SEQ ID NO: 187); (21) LAHQDATKNA (SEQ ID NO: 188); (22) LAHQDTKKPA (SEQ ID NO: 189); (23) ILSETTRPA (SEQ ID NO: 190); (24) LAHQDTTKKC (SEQ ID NO: 191); (25) LALGEATRPA (SEQ ID NO: 192); (26) LALGETTRTA (SEQ ID NO: 193); (27) LALSEATRPA (SEQ ID NO: 194); (28) LAKDETKNSA (SEQ ID NO: 195); (29) LALGETTKPA (SEQ ID NO: 196); and (30) LAHQATTKNA (SEQ ID NO: 197). Thus, in a further embodiment, the variant capsid polypeptide has a sequence selected from the group consisting of SEQ ID NOs: 168-197.

In some embodiments, the rAAV virion can comprise a deletion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids in a capsid protein relative to a corresponding parental AAV capsid protein. In some embodiments, the rAAV virion can comprise a deletion of at least 20, at least 25, at least 30, at least 35, at least 40, at least 45, at least 50, at least 55, at least 60, at least 65, at least 70, at least 75, at least 80, at least 85, at least 90, at least 95 or at least 100 amino acids in a capsid protein. In some embodiments, the rAAV virion can comprise a deletion of at most about 100 amino acids, at most about 200, at most about 300, or at most about 400 amino acids in a capsid protein. In some embodiments, the rAAV virion can comprise a deletion of from about 1 to about 100, from about 1 to about 90, from about 1 to about 80, from about 1 to about 70, from about 1 to about 60, from about 1 to about 50, from about 1 to about 40, from about 1 to about 30, from about 1 to about 20, from about 1 to about 15, from about 1 to about 10, or from about 1 to about 5 amino acids in a capsid protein. In some embodiments, the rAAV virion can comprise a deletion of from about 5 amino acids to about 20 amino acids, from about 5 amino acids to about 19 amino acids, from about 5 amino acids to about 18 amino acids, from about 5 amino acids to about 17 amino acids, from about 5 amino acids to about 16 amino acids, from about 5 amino acids to about 15 amino acids, from about 5 amino acids to about 14 amino acids, from about 5 amino acids to about 15 amino acids, from about 5 amino acids to about 12 amino acids, from about 5 amino acids to about 11 amino acids, from about 5 amino acids to about 10 amino acids, from about 5 amino acids to about 9 amino acids, front about 5 amino acids to about 8 amino acids, from about 5 amino acids to about 7 amino acids, or from about 5 amino acids to about 6 amino acids in a capsid protein.

In some embodiments, the rAAV virion can comprise an insertion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids in a capsid protein relative to a corresponding parental AAV capsid protein. In some embodiments, the rAAV virion can comprise an insertion of at least 20, at least 25, at least 30, at least 35, at least 40, at least 45, at least 50, at least 55, at least 60, at least 65, at least 70, at least 75, at least 80, at least 85, at least 90, at least 95 or at least 100 amino acids in a capsid protein. In some embodiments, the rAAV virion ran comprise an insertion of at most about 100 amino acids, at most about 200, at most about 300, or at most about 400 amino acids in a capsid protein. In some embodiments, the rAAV virion can comprise an insertion of from about 1 to about 100, from about 1 to about 90, from about 1 to about 80, from about 1 to about 70, from about 1 to about 60, from about 1 to about 50, from about 1 to about 40, from about 1 to about 30, from about 1 to about 20, from about 1 to about 15, from about 1 to about 10, or from about 1 to about 5 amino acids in a capsid protein. In some embodiments, the rAAV virion can comprise an insertion of from about 5 amino acid acids to about 20 amino acids, from about 5 amino acids to about 19 amino acids, from about 5 amino acids to about 18 amino acids, from about 5 amino acids to about 17 amino acids, from about 5 amino acids to about 16 amino acids, from about 5 amino acids to about 15 amino acids, from about 5 amino acids to about 14 amino acids, from about 5 amino acids to about 13 amino acids, from about 5 amino acids to about 12 amino acids, from about 5 amino acids to about 11 amino acids, from about 5 amino acids to about 10 amino acids, from about 5 amino acids to about 9 amino acids, from about 5 amino acids to about 8 amino acids, from about 5 amino acids to about 7 amino acids, or from about 5 amino acids to about 6 amino acids in a capsid protein.

In some embodiments, the rAAV virion can comprise a substitution of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids in a capsid protein relative to a corresponding parental AAV capsid protein. In some embodiments, the rAAV virion can comprise a substitution of from about 1 amino acids to about 20 amino acids, from about 1 amino acids to about 19 amino acids, from about 1 amino acids to about 18 amino acids, from about 1 amino acids to about 17 amino acids, from about 1 amino acids to about 16 amino acids, from about 1 amino acids to about 15 amino acids, from about 1 amino acids to about 14 amino acids, from about 1 amino acids to about 13 amino acids, from about 1 amino acids to about 12 amino acids, from about 1 amino acids to about 11 amino acids, from about 1 amino acids to about 10 amino acids, from about 1 amino acids to about 9 amino acids, from about 1 amino acids to about 8 amino acids, from about 1 amino acids to about 7 amino acids, from about 1 amino acids to about 6 amino acids, from about 1 to about 5 amino acids, from about 1 to about 4 amino acids, from about 1 to about 3 amino acids, or from about 1 to about 2 amino acids in a capsid protein.

In some embodiments, the rAAV virion can comprise at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 total amino acid insertions, deletions or substitutions in a capsid protein relative to a corresponding parental, unmodified capsid protein. In some embodiments, the rAAV virion can comprise at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 total amino acid insertions, deletions or substitutions in a capsid protein relative to a corresponding parental, unmodified capsid protein. In some embodiments, the rAAV virion can comprise at least about 100, at least about 200, at least about 300, or at least about 400 total amino acid insertions, deletions or substitutions in a capsid protein relative to a corresponding parental, unmodified capsid protein.

In some embodiments, the rAAV virion comprises a variant capsid protein with an amino acid sequence that is at least about 50%, at least about 55%, at least about 60%, at least about 65%, at least about 70%, at least about 80%, at least about at 85%, at least about 86%, at least about 87%, at least about 88%, at least about 89%, at least about 90%, at least about 91%, at least about 92%; at least about 93%, at least about 94%, at least about 95%, at least about 96%, at least about 97%, at least about 98%, or at least about 99% homologous to a capsid protein of a parental, unmodified AAV capsid protein.

In some cases, the modification can be after amino acid 587 of AAV2, or the corresponding residue of a capsid subunit of another AAV serotype. It should be noted that the residue 587 is based on an AAV2 capsid protein. A modification can also be incorporated at a corresponding site in an AAV serotype other than AAV2 (e.g., AAV8, AAV9, etc.). Those skilled in the art would know, based on a comparison of the amino acid sequences of capsid proteins of various AAV serotypes, where a modification site corresponding to amino acid 587 of AAV2 would be in a capsid protein of any given AAV serotype. See, e.g., GenBank Accession No. NP_049542 for AAV1; GenBank Accession No. AAD13756 for AAV5; GenBank Accession No. AAB95459 for AAV6; GenBank Accession No. YP_077178 for AAV7; GenBank Accession No. YP_077180 for AAV8; GenBank Accession No AAS99264 for AAV9 and GenBank Accession No. AAT46337 for AAV10.

In some embodiments, an amino acid modification of a capsid protein described herein can confer an increase in infectivity of an ocular cell compared to the infectivity of the retinal cell by an AAV virion comprising the corresponding parental or unmodified AAV capsid protein. In some cases, the ocular cell can be a retinal ganglion cell (RGC). In some cases, the retinal cell can be a retinal pigment epithelium (RPE) cell. In some cases, the ocular cell can be a Muller cell. In some cases, the ocular cell can be an astrocyte. In some cases, the retinal cells can include amacrine cells, bipolar cells, or horizontal cells. Viral vectors for use in the disclosure can include those that exhibit low toxicity and/or low immunogenicity in a subject and express therapeutically effective quantities of a MW-opsin transgene in a subject, e.g., human patient.

In some embodiments, the increase in retinal cell infectivity of rAAV variant is at least 5%, at least 10%, at least 20%, at least 30%, at least 40%, at least 50%, at least 60%, at least 70%, at least 80%, at least 90%, or at least 100% as compared to an AAV virion comprising the corresponding parental or unmodified AAV capsid protein. In some embodiments, the increase in infectivity of animal cells is an increase of between 5% to 100%, between 5% to 95%, between 5% to 90%; between 5% to 85%, between 5% to 80%, between 5% to 75%, between 5% to 70%, between 5% to 65%, between 5% to 60%, between 5% to 55%, between 5% to 50%, between 5% to 45%, between 5% to 40%, between 5% to 35%, between 5% to 30%, between 5% to 25%, between 5% to 20%, between 5% to 15%, between 5% to 10% as compared to an AAV virion comprising the corresponding parental or unmodified AAV capsid protein.

In some embodiments, the increase in retinal cell infectivity of a rAAV variant is at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, or at least 2-fold compared to an AAV virion comprising the corresponding parental or unmodified AAV capsid protein. In some embodiments, the increase in infectivity is at least 2-fold, at least 3-fold, at least 4-fold, at least 5-fold, at least 6-fold, at least 7-fold, at least 8-fold, at least 9-fold, or at least 10-fold as compared to an AAV virion comprising the corresponding parental AAV capsid protein. In some embodiments, the increase in infectivity is at least 15-fold, at least 20-fold, at least 25-fold, at least 30-fold, at least 35-fold, at least 40-fold, at least 45-fold, at least 50-fold, at least 55-fold, at least 60-fold, at least 65-fold, at least 70-fold, at least 75-fold, at least 80-fold, at least 85-fold, at least 90-fold, or at least 100-fold compared to an AAV virion comprising the corresponding parental or unmodified AAV capsid protein.

In some embodiments, the increase in retinal cell infectivity is between 10-fold to 100-fold, between 10-fold to 95-fold, between 10-fold to 90-fold, between 10-fold to 85-fold, between 10-fold to 80-fold, between 10-fold to 75-fold, between 10-fold to 70-fold, between 10-fold to 65-fold, between 10-fold to 60-fold, between 10-fold to 55-fold, between 10-fold to 50-fold, between 10-fold to 45-fold, between 10-fold to 40-fold, between 10-fold to 35-fold, between 10-fold to 30-fold, between, 10-fold to 25-fold, between 10-fold to 20-fold, or between 10-fold to 15-fold as compared to an AAV virion comprising the corresponding parental or unmodified AAV capsid protein.

In some embodiments, the increase in retinal cell infectivity is between 2-fold to 20-fold, between 2-fold to 19-fold, between 2-fold to 18-fold, between 2-fold to 17-fold, between 2-fold to 16-fold, between 2-fold to 15-fold, between 2-fold to 14-fold, between 2-fold to 13-fold, between 2-fold to 12-fold, between 2-fold to 11-fold, between 2-fold to 10-fold, between 2-fold to 9-fold, between 2-fold to 8-fold, between 2-fold to 7-fold, between 2-fold to 6-fold, between 2-fold to 5-fold, between 2-fold to 4-fold, or between 2-fold to 3-fold as compared to an AAV virion comprising the corresponding parental or unmodified AAV capsid protein.

In some embodiments, an amino acid modification of a capsid protein described herein can confer an increase in an ability to cross an internal limiting membrane (ILM) in an eye of a primate or human subject as compared to the ability of an AAV virion comprising the corresponding parental or unmodified AAV capsid protein to cross the ILM in the eye of the subject. In some embodiments, the increase in the ability to cross the ILM is an increase of at least 5%, at least 10%, at least 20%, at least 30%, at least 40%, at least 50%, at least 60%, at least 70%, at least 80%, at least 90%, or at least 100% as compared to an AAV virion comprising the corresponding parental or unmodified AAV capsid protein. In some embodiments, the increase in the ability to cross the ILM is an increase of between 5% to 100%, between 5% to 95%, between 5% to 90%, between 5% to 85%, between 5% to 80%, between 5% to 75%, between 5% to 70%, between 5% to 65%, between 5% to 60%, between 5% to 55%, between 5% to 50%, between 5% to 45%, between 5% to 40%, between 5% to 35%, between 5% to 30%, between 5% to 25%, between 5% to 20%, between 5% to 15%, or between 5% to 10% as compared to the parental or unmodified AAV capsid protein.

In some embodiments, the increase in the ability to cross the ILM is at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, or at least 2-fold compared to an AAV virion comprising the corresponding parental AAV capsid protein. In some embodiments, the increase in the ability to cross the ILM is at least 2-fold, at least 3-fold, at least 4-fold, at least 5-fold, at least 6-fold, at least 7-fold, at least 8-fold, at least 9-fold, or at least 10-fold as compared to an AAV virion comprising the corresponding parental AAV capsid protein. In some embodiments, the increase in the ability to cross the ILM is at least 15-fold, at least 20-fold, at least 25-fold, at least 30-fold, at least 35-fold, at least 40-fold, at least 45-fold, at least 50-fold, at least 55-fold, at least 60-fold, at least 65-fold, at least 70-fold, at least 75-fold, at least 80-fold, at least 85-fold, at least 90-fold, or at least 100-fold compared to an AAV virion comprising the corresponding parental or unmodified AAV capsid protein.

In some embodiments, the increase in the ability to cross the TLM is between 10-fold to 100-fold, between 10-fold to 95-fold, between 10-fold to 90-fold, between 10-fold to 85-fold, between 10-fold to 80-fold, between 10-fold to 75-fold, between 10-fold to 70-fold, between 10-fold to 65-fold, between 10-fold to 60-fold, between 10-fold to 55-fold, between 10-fold to 50-fold, between 10-fold to 45-fold, between 10-fold to 40-fold, between 10-fold to 35-fold, between 10-fold to 30-fold, between, 10-fold to 25-fold, between 10-fold to 20-fold, or between 10-fold to 15-fold as compared to an AAV virion comprising the corresponding parental or unmodified AAV capsid protein.

In some embodiments, the increase in the ability to cross the ILM is between 2-fold to 20-fold, between 2-fold to 19-fold, between 2-fold to 18-fold, between 2-fold to 17-fold, between 2-fold to 16-fold, between 2-fold to 15-fold, between 2-fold to 14-fold, between 2-fold to 13-fold, between 2-fold to 12-fold, between 2-fold to 11-fold, between 2-fold to 10-fold, between 2-fold to 9-fold, between 2-fold to 8-fold, between 2-fold to 7-fold, between 2-fold to 6-fold, between 2-fold to 5-fold, between 2-fold to 4-fold, or between 2-fold to 3-fold as compared to an AAV virion comprising the corresponding parental or unmodified AAV capsid protein.

In some embodiments, the viral vector of the disclosure is measured as vector genomes. In some cases, a unit dose of recombinant viruses of this disclosure comprise between 1×10¹⁰ to 2×10¹⁰, between 2×10¹⁰ to 3×10¹⁰, between 3×10¹⁰ to 4×10¹⁰, between 4×10¹⁰ to 5×10¹⁰, between 5×10¹⁰ to 6×10¹⁰, between 6×10¹⁰ to 7×10¹⁰, between 7×10¹⁰ to 8×10¹⁰, between 8×10¹⁰ to 9×10¹⁰, between 9×10¹⁰ to 10×10¹⁰, between 1×10¹¹ to 2×10¹¹, between 2×10¹¹ to 3×10¹¹, between 3×10¹¹ to 4×10¹¹, between 4×10¹¹ to 5×10¹¹, between 5×10¹¹ to 6×10¹¹, between 6×10¹¹ to 7×10¹¹, between 7×10¹¹ to 8×10¹¹, between 8×10¹¹ to 9×10¹¹, between 9×10¹¹ to 10×10¹¹, between 1×10¹² to 2×10¹², between 2×10¹² to 3×10¹², between 3×10¹² to 4×10¹², between 4×10¹² to 5×10¹², between 5×10¹² to 6×10¹², between 6×10¹² to 7×10¹², between 7×10¹² to 8×10¹², between 8×10¹² to 9×10¹², between 9×10¹² to 10×10¹², between 1×10¹³ to 2×10¹³, between 2×10¹³ to 3×10¹³, between 3×10¹³ to 4×10¹³, between 4×10¹³ to 5×10¹³, between 5×10¹³ to 6×10¹³, between 6×10¹³ to 7×10¹³, between 7×10¹³ to 8×10¹³, between 8×10¹³ to 9×10¹³, or between 9×10¹³ to 10×10¹³ vector genomes. In some embodiments, the rAAV of this disclosure is between 10¹⁰ to 10¹³, between 10¹⁰ to 10¹⁴, between 2×10¹¹ to 4×10¹¹, between 3×10¹¹ to 5×10¹¹, between 4×10¹¹ to 6×10¹¹, between 5×10¹¹ to 7×10¹¹, between 6×10¹¹ to 8×10¹¹, between 7×10¹¹ to 9×10¹¹, between 8×10¹¹ to 10×10¹¹, between 1×10¹² to 3×10¹², between 2×10² to 4×10¹, between 3×10² to 5×10¹, between 4×10² to 6×10¹, between 5×10¹² to 7×10¹², between 6×10¹² to 8×10¹², between 7×10¹² to 9×10¹², between 8×10¹² to 10×10¹², between 1×10¹³ to 5×10¹³, between 5×10¹³ to 10×10¹³, between 10¹² to 5×10¹², or between 5×10¹² to 10×10¹² vector genomes.

In some cases, recombinant viruses of this disclosure are of about 1×E10, about 1.5×E10, about 2×E10, about 2.5×E10, about 3×E10, about 3.5×E10, about 4×E10, about 4.5×E10, about 5×E10, about 5.5×E10, about 6×E10, about 6.5×E10, about 7×E10, about 7.5×E10, about 8×E10, about 8.5×E10, about 9×E10, about 9.5×E10, about 10×E10, about 1×E11, about 1.5×E11, about 2×E11, about 2.5×E11, about 3×E11, about 3.5×E11, about 4×E11, about 4.5×E11, about 5×E11, about 5.5×E11, about 6×E11, about 6.5×E11, about 7×E11, about 7.5×E11, about 8×E11, about 8.5×E11, about 9×E11, about 9.5×E11, about 10×E11, about 1×E12, about 1.3×E12, about 1.5×E12, about 2×E12, about 2.1×E12, about 2.3×E12, about 2.5×E12, about 2.7×E12, about 2.9×E12, about 3×E12, about 3.1×E12, about 3.3×E12, about 3.5×E12, about 3.7×E12, about 3.9×E12, about 4×E12, about 4.1×E12, about 4.3×E12, about 4.5×E12, about 4.7×E12, about 4.9×E12, about 5×E12, about 5.1×E12, about 5.3×E12, about 5.5×E12, about 5.7×E12, about 5.9×E12, about 6×E12, about 6.1×E12, about 6.3×E12, about 6.5×E12, about 6.7×E12, about 6.9×E12, about 7×E12, about 7.1×E12, about 7.3×E12, about 7.5×E12, about 7.7×E12, about 7.9×E12, about 8×E12, about 8.1×E12, about 8.3×E12, about 8.5×E12, about 8.7×E12, about 8.9×E12, about 9×E12, about 9.1×E12, about 9.3×E12, about 9.5×E12, about 9.7×E12, about 9.9×E12, about 10×E12, about 10.1×E12, about 10.3×E12, about 10.5×E12, about 10.7×E12, about 10.9×E12, about 11×E12, about 11.5×E12, about 12×E12, about 12.5×E12, about 13×E12, about 13.5×E12, about 14×E12, about 14.5×E12, about 15×E12, about 15.5×E12, about 16×E12, about 16.5×E12, about 17×E12, about 17.5×E12, about 18×E12, about 18.5×E12, about 19×E12, about 19.5×E12, about 20×E12, about 20.5×E12, about 30×E12, about 30.5×E12, about 40×E12, about 40.5×E12, about 50×E12, about 50.5×E12, about 60×E12, about 60.5×E12, about 70×E12, about 70.5×E12, about 80×E12, about 80.5×E12, about 90×E12, about 95×E12, or about 100×E12 vector genomes, wherein E is a short-hand for base 10 for exponentiation, and xEy refers to x multiplied by 10 to the y power/exponent. In some embodiments, the recombinant viruses comprise 1×E13 vector genomes.

In some embodiments, pharmaceutical compositions disclosed herein comprise recombinant viruses of at least 5×E11, at least 5.5×E11, at least 6×E11, at least 6.5×E11, at least 7×E11, at least 7.5×E11, at least 8×E11, at least 8.5×E11, at least 9×E11, at least 9.5×E11, at least 10×E11, at least 1×E12, at least 1.31×E12, at least 1.51×E12, at least 2×E12, at least 2.1×E12, at least 2.3×E12, at least 2.5×E12, at least 2.7×E12, at least 2.9×E12, at least 3×E12, at least 3.1×E12, at least 3.3×E12, at least 3.5×E12, at least 3.7×E12, at least 3.9×E12, at least 4×E12, at least 4.1×E12, at least 4.3×E12, at least 4.5×E12, at least 4.7×E12, at least 4.9×E12, at least 5×E12, at least 5.1×E12, at least 5.3×E12, at least 5.5×E12, at least 5.7×E12, at least 5.9×E12, at least 6×E12, at least 6.1×E12, at least 6.3×E12, at least 6.5×E12, at least 6.7×E12, at least 6.9×E12, at least 7×E12, at least 7.1×E12, at least 7.1×E12, at least 7.3×E12, at least 7.5×E12, at least 7.7×E12, at least 7.9×E12, at least 8×E12, at least 8.1×E12, at least 8.3×E12, at least 8.5×E12, at least 8.7×E12, at least 8.9×E12, at least 9×E12, at least 9.1×E12, at least 9.1×E12, at least 9.3×E12, at least 9.5×E12, at least 9.7×E12, at least 9.9×E12, at least 10×E12, at least 10.1×E12, at least 10.3×E12, at least 10.5×E12, at least 10.7×E12, at least 10.9×E12, at least 11×E12, at least 11.5×E12, at least 12×E12, at least 12.5×E12, at least 13×E12, at least 13.5×E12, at least 14×E12, at least 14.5×E12, at least 15×E12, at least 15.5×E12, at least 16×E12, at least 16.5×E12, at least 17×E12, at least 17.5×E12, at least 18×E12, at least 18.5×E12, at least 19×E12, at least 19.5×E12, at least 20×E12, at least 20.5×E12, at least 30×E12, at least 30.5×E12, at least 40×E12, at least 40.5×E12, at least 50×E12, at least 50.5×E12, at least 60×E12, at least 60.5×E12, at least 70×E12, at least 70.5×E12, at least 80×E12, at least 80.5×E12, at least 90×E12, at least 95×E12, or at least 100×E12 vector genomes, wherein E is a short-hand for base 10 for exponentiation, and wherein xEy refers to x multiplied by base 10 to the y power/exponent. In some embodiments, pharmaceutical composition disclosed herein comprise recombinant viruses of about 1×E13 vector genomes.

In some embodiments, the viral vector of the disclosure is measured using multiplicity of infection (MOI). In some cases, MOI refers to the ratio, or multiple of vector or viral genomes to the cells to which the polynucleotide can be delivered. In some cases, the MOI is 1×10⁶. In some cases, recombinant viruses of the disclosure can be at least 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷ and 1×10¹⁸ MOI. In some cases, recombinant viruses of this disclosure can be from 1×10⁸ to 1×10¹⁵ MOI. In some cases, recombinant viruses of the disclosure can be at most 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI.

In some embodiments, the polynucleotide may be delivered without the use of a virus (i.e., with a non-viral vector), and may be measured as the quantity of polynucleotide. Generally, any suitable amount of polynucleotide may be used with the pharmaceutical compositions and methods of this disclosure.

In some embodiments, a self-complementary vector (sc) can be used. The use of self-complementary AAV vectors may bypass the requirement for viral second-strand DNA synthesis and may lead to greater rate of expression of the transgene protein, as provided by Wu, Hum Gene Ther. 2007, 18(2):171-82, incorporated by reference herein.

In some embodiments, the vector can be a retroviral vector. Retroviral vectors can include Moloney murine leukemia viruses and HIV-based viruses. In some embodiments, a HIV-based viral vector can be used, wherein the HIV-based viral vector comprises at least two vectors wherein the gag and pol gens are from an HIV genome and the env gene is from another virus. In some embodiments, DNA viral vectors may be used. These vectors can include pox vectors such as orthopox or avipox vectors, herpesvirus vectors such as a herpes simples I virus (HSV) vector [Geller, A. I. et al., J. Neurochem, 64:487 (1995); Lim, F., et al., in DNA Cloning: Mammalian Systems, D. Glover, Ed. (Oxford Univ. Press. Oxford England) (1995); Geller. A. I. et al., Proc Natl. Acad, Sci.: U.S.A.; 90:7603 (1993); Geller, A. I., et al., Proc Natl. Acad. Sci. USA: 87; 1149 (1990)], Adenovirus Vectors [LeGal LaSalle et al., Science. 259:988 (1993); Davidson, et al., Nat. Genet. 3:219(1993); Yang, et al., J. Virol 69:2004 (1995)] and Adeno-associated Virus Vectors [Kaplitt, M. G. et al., Nat Genet. 8:148 (1994)], incorporated by reference here in their entirety.

In some embodiments, the vector can be a lentiviral vector. Lentiviral vectors for use in the disclosure may be derived from human and non-human (including SIV) lentiviruses. Examples of lentiviral vectors can include polynucleotide sequences required for vector propagation as well as a tissue-specific promoter operably linked to MW-opsin transgene. Polynucleotide sequences may include the viral LTRs, a primer binding site, a polypurine tract, att sites, and an encapsidation site.

In some embodiments, the vector can be an alphavirus vector. Alphavirus-based vectors such as those made from semliki forest virus (SFV) and sindbis virus (SIN) may also be used in the disclosure. Use of alphaviruses is described in Lundstrom, K., Intervirology 43:247-257, 2000 and Perri et al., Journal of Virology 74:9802-9807, 2000, incorporated by reference herein in their entirety.

In some embodiments, the vector can be a pox viral vector. Pox viral vectors may introduce a gene into the cell's cytoplasm. Avipox virus vectors may result in only a short term expression of the gene or polynucleotide. Adenovirus vectors, adeno-associated virus vectors and herpes simplex virus (HSV) vectors may be used with the compositions and methods of the disclosure. The adenovirus vector may result in a shorter term expression (e.g., less than about a month) than adeno-associated virus, in some aspects, and may exhibit much longer expression. The particular vector chosen may depend upon the target cell and the condition being treated.

In some embodiments, vectors, e.g., naked DNA or a plasmid, can be delivered into a cell, tissue, or subject using micelles: microemulsions; liposomes; nanospheres; nanoparticles; nanocapsules; solid lipid nanoparticles; dendrimers; polyethylenimine derivative and single-walled carbon nanotubes; and other macromolecular complexes capable of mediating delivery of a polynucleotide to a target cell. In some cases, a vector may be an organic or inorganic molecule. In some cases, a vector is a small molecule (i.e., <5 kD), or a macromolecule (i.e., >5 kD).

Also disclosed herein are recombinant adeno-associated virus (rAAV) virions adapted for gene therapy for restoring or enhancing visual function comprising: (a) a variant AAV capsid protein, wherein the variant capsid protein confers an increase in an infectivity of retinal cells relative to an AAV virion that comprises a corresponding non-variant or unmodified AAV capsid protein; (b) a heterologous polynucleotide sequence encoding a MW-opsin polypeptide or therapeutic transgene. In some embodiments, the rAAV used for gene therapy is rAAV2.

In some embodiments, disclosed herein are recombinant adeno-associated virus (rAAV) virions for restoring or enhancing visual function comprising: (a) a variant AAV capsid protein, where the variant AAV capsid protein comprises an amino acid modification in a solvent-exposed region of the capsid protein and shows an increased infectivity of retinal cells relative to a corresponding non-variant AAV capsid protein; and (b) a heterologous polynucleotide comprising a polynucleotide sequence encoding MW-opsin, and where an administration of an effective amount of the rAAV by intraocular, intravitreal or subretinal injection in an eye of a primate or human subject results in restoring or enhancing visual function in the eye.

Also disclosed herein are recombinant adeno-associated virus (rAAV) virions for restoring or enhancing visual function comprising: (a) a variant AAV capsid protein, where the variant AAV capsid protein comprises an amino acid modification in a solvent-exposed region of the AAV capsid protein, and wherein the variant capsid protein confers an increased ability to cross an internal limiting membrane (ILM) in an eye; and (b) a heterologous polynucleotide comprising a polynucleotide sequence encoding MW-opsin, and where an administration of an effective amount of the rAAV by intraocular, intravitreal, or subretinal injection in an eye of a primate or human subject results in restoring or enhancing visual function in the eye.

Also disclosed herein are gene therapy compositions in a unit dose form for treating an ocular condition or disease, comprising: (a) a recombinant adeno-associated virus (rAAV) virion comprising: (i) a variant AAV capsid protein, wherein the variant AAV capsid protein comprises an amino acid modification in a solvent-exposed region of the capsid protein and shows an increased infectivity of retinal cells relative to a corresponding non-variant AAV capsid protein; and (ii) a heterologous polynucleotide comprising a polynucleotide sequence encoding MW-opsin, wherein the MW-opsin when transduced restores or enhances visual function in an eye of a primate or human subject; and (b) a pharmaceutically acceptable excipient; where the rAAV virion is in an amount sufficient to at least partially restore or enhance visual function when administered by intraocular, intravitreal, or subretinal injection in the eye of the primate as a unit dose.

### Therapeutic Agents

In some embodiments, a gene therapy is used to deliver a therapeutic transgene having MW-opsin activity that is suitable for or adapted for administration to an eye or vitreous of an eye of a non-human primate or a human subject. In some embodiments, rAAV comprising a capsid variant described herein comprises a heterologous polynucleotide sequence that encodes MW-opsin is used to deliver the sequence of the MW-opsin transgene into retinal cells upon intraocular, intravitreal, or subretinal injection to a subject. In some embodiments, the rAAV comprising the MW-opsin transgene is formulated for gene therapy and intravitreal injection. In some embodiments, the MW-opsin transgene refers to a functional fragment or a variant thereof. In some embodiments, the polynucleotide sequence of the MW-opsin transgene is SEQ ID NO: 3. In some embodiments, the polynucleotide sequence and/or the amino acid sequence the MW-opsin transgene (e.g., SEQ ID NO: 3) is further modified to enhance its activity, expression, stability, and/or solubility in vivo.

In some embodiments, the MW-opsin transgene polynucleotide sequence used in a gene therapy or rAAV disclosed herein is compared to the corresponding polynucleotide sequence of SEQ ID NO: 3, and shows at least 75%, 80%, 85%, 90%, 91%, 92%, 93%, 94%, 95%, 96%, 97%, 98%, 99%, 99.9%, 99.99% or 100% sequence homology. In some embodiments, the polynucleotide sequence used in a gene therapy or rAAV disclosed herein is compared to the polynucleotide sequence of SEQ ID NO: 8 or SEQ ID NO: 9, and shows at least 75%, 80%, 85%, 90%, 91%, 92%, 93%, 94%, 95%, 96%, 97%, 98%, 99%, 99.9%, 99.99% or 100% sequence homology.

The present disclosure contemplates methods and pharmaceutical compositions as disclosed herein comprising one or more therapeutic agents. In some embodiments, the therapeutic agent is a MW-opsin polypeptide. In some embodiments, the MW-opsin polypeptide is expressed from a rAAV vector or gene therapy, which is delivered into a target cell, tissue, or a subject in vivo. Gene therapy has the advantage of providing the therapeutic agent, e.g., MW-opsin polypeptide, for a prolonged period of time in vivo, which decreases the need for repeated injections as compared to administration of a protein-based therapy. Such advantage of gene therapy can lead to a more sustained delivery and expression of the therapeutic agent in vivo, which provides an improvement over the current standard of care. Additionally, a gene therapy can also provide a more targeted delivery of the therapeutic agent in vivo, e.g., to target cells, and minimize off-target effects. In some embodiments, the gene product disclosed herein is a MW-opsin polypeptide that when expressed can result in restoring or enhancing visual function in an eye of a subject. In some embodiments, a gene product disclosed herein can be a MW-opsin polypeptide or a fragment thereof that can restore or enhance visual function in an individual.

### Indications

In some cases, rAAV virion of any serotype comprising a variant capsid protein and a therapeutic transgene, or a pharmaceutical composition thereof as described herein, can at least partially ameliorate an eye condition or disease associated with loss of rod and cone photoreceptors. In some embodiments, a rAAV virion comprising a capsid variant protein is used to deliver a MW-opsin transgene into an eye of a human subject. Individuals suitable for treatment with a method of the present disclosure include individuals having a retinal degeneration condition in which the natural light sensitivity is lost and vision is therefore compromised, but where neurons late in the retinal circuit (e.g. bipolar cells or amacrine interneurons or ganglion cells that output to the brain) are spared and can be made directly sensitive to light by introduction of the cone opsin(s).

Indications described herein include geographic atrophy, age-related macular degeneration (AMD), macular edema following retinal vein occlusion (RVO), diabetic macular edema (DME), retinal vein occlusion, and diabetic retinopathy (DR) in patients with DME. Other embodiments described herein include retinitis pigmentosa, macular degeneration, retinoschisis, Leber's Congenital Amaurosis, cone rod dystrophies, Stargardt disease, Bardet Biedl syndrome, choroideremia, Usher syndrome, and Bietti crystalline dystrophy. In some cases, methods and pharmaceutical compositions disclosed herein can be used to prevent or treat an eye condition or disease for which a MW-opsin transgene is approved or indicated for. In some embodiments, a gene therapy (e.g., rAAV based gene therapy) is used to treat or prevent an eye condition or disease that is responsive to at least one current standard of care for the eye condition/disease, including, but not limited to, AMD, macular edema following RVO, DME, and diabetic retinopathy in patients with DME. In some embodiments, a rAAV gene therapy is used to treat or prevent any eye condition or disorder characterized by loss of rod and cone photoreceptors. In another aspect, the present disclosure provides pharmaceutical compositions provided herein for the treatment of diseases such as, for example: AMD, retinitis pigmentosa, macular degeneration, retinoschisis, Leber's Congenital Amaurosis, cone rod dystrophies, Bardet Biedl syndrome, choroideremia, Usher syndrome, Bietti crystalline dystrophy, and the like. In some embodiments, the eye condition can be retinitis pigmentosa. In other embodiments, the individual has experienced retinal detachment or photoreceptor loss due to trauma, head injury, or as a complication of another disease (e.g., diabetic retinopathy).

In some embodiments, the eye condition can be AMD. AMD can create deterioration of central vision. Other symptoms which can occur include color disturbances, and metamorphopsia (distortions in which straight lines appears wavy). In some embodiments, methods and pharmaceutical compositions as disclosed herein are used to treat AMD. The term “AMD,” if not otherwise specified, can be either dry AMD or wet AMD. The present disclosure contemplates treatment or prevention of AMD, wet AMD and/or dry AMD. In some embodiments, methods and pharmaceutical compositions as disclosed herein are used to treat AMD.

### Methods of Use

In some embodiments, the present disclosure provides a method for treating an ocular disease, comprising administering a pharmaceutically effective amount of the pharmaceutical compositions provided herein to a human subject in need of such treatment. In some embodiments, the disease is selected from the group of ocular diseases including retinitis pigmentosa, macular degeneration, geographic atrophy, age-related macular degeneration, retinoschisis, Leber's Congenital Amaurosis, cone rod dystrophies, Bardet Biedl syndrome, choroideremia, Usher syndrome, Bietti crystalline dystrophy, and other inherited retinal dystrophies such as Stargardt disease. In other embodiments, the individual has experienced retinal detachment or photoreceptor loss due to trauma, head injury, or as a complication of another disease (e.g., diabetic retinopathy). For example, in some cases, the individual has experienced retinal detachment resulting from blunt trauma, such as a blast injury (e.g., in a military battle), or resulting from an impact to the head, e.g., in the course of an auto accident or other accident resulting in impact to the head. In some instances, the photoreceptors are lost due to traumatic detachment of the retina from the underlying RPE, but the inner retinal neurons are intact. Individuals suitable for treatment with a method of the present disclosure include individuals having photoreceptor loss due to acute light damage, laser exposure, or chemical toxicity.

In some embodiments, pharmaceutical compositions comprising a rAAV comprising a variant capsid protein (e.g., rAAV.7m8) and a polynucleotide sequence that encodes a MW-opsin polypeptide is used to treat or prevent AMD, including dry AMD and wet AMD. In some embodiments, pharmaceutical compositions comprising a rAAV comprising a variant capsid protein (e.g., rAAV.7m8) and a polynucleotide sequence that encodes MW-opsin polypeptide is used to treat or prevent retinitis pigmentosa, macular degeneration, retinoschisis, Leber's Congenital Amaurosis, cone rod dystrophies, Bardet Biedl syndrome, choroideremia, Usher syndrome, and Bietti crystalline dystrophy.

In some cases, risks are lower in gene therapy because it requires only one injection in a patient's lifetime, or is given not more than once in at least 2, 5, 10, 20, 30, 40, or 50 years. In some cases, treatment with the MW-opsin gene therapy as disclosed herein can be more cost-effective than protein-based injections because a gene therapy's therapeutic effects can last longer and the cost of a one-time gene therapy injection may be lower than the combined cost of multiple, repeated injections of a protein.

Also, by not requiring repeated injections, gene therapy addresses the patient compliance and adherence challenge associated with the therapies that require repeated injections, as non-compliance (e.g., when a patient forgets or misses one or more scheduled injection) can result in vision loss and deterioration of the eye disease or condition. The rate of non-compliance and non-adherence to treatment regimens that require repeated or frequent trips to medical offices for administration is higher among elderly patients, who are most impacted by AMD. Therefore, delivering a MW-opsin transgene into an eye of a patient via gene therapy, e.g., as a one-time intravitreal injection, can provide a more convenient treatment option for patients and improve patient outcomes by addressing the non-compliance and non-adherence problem.

In some embodiments, method of use of the MW-opsin gene therapy described herein includes reconstituting a lyophilized form of the pharmaceutical composition described herein (e.g., rAAV comprising a MW-opsin polynucleotide sequence) according to the drug label and administering said reconstituted MW-opsin gene therapy to a subject or human patient.

In some embodiments, rAAV-MW-opsin virion can be administered via intraocular injection, by intravitreal injection, by subretinal injection, or by any other convenient mode or route of administration into an eye of an individual. Other convenient modes or routes of administration can include, e.g., topical, eye drops, periocular, intraocular, intravitreal, subconjunctive, retrobulbar, into the sclera, and intercameral etc. In some embodiments, methods and pharmaceutical compositions disclosed herein involve administration by intravitreal injection.

A “therapeutically effective amount” as described herein can be a relatively broad range that can be determined through clinical trials. For injection directly into the eye or intravitreal injection, a therapeutically effective dose can be on the order of from 10¹⁰ to 10¹³ vector genomes of MW-opsin gene therapy.

Also disclosed herein are methods of treating an eye condition or disease comprising administering a rAAV virion adapted for gene therapy and in vivo delivery of a polynucleotide sequence for expressing MW-opsin, as described herein to an eye of a human subject; where the human subject has been previously diagnosed with an eye condition associated with a retinal condition in which the natural light sensitivity is lost and is vision is therefore compromised. In some embodiments, the gene therapy is administered to a patient pretreated with at least one of the approved therapies, e.g., anti-VEGF agent. In some embodiments, the gene therapy disclosed herein is administered to a patient who was pre-treated with at least one of the approved therapies, e.g., anti-VEGF agent injections, and failed to show improvement. In some embodiments, patients who receive the gene therapy disclosed herein have one or more risk factors that disfavor treating the patient with therapies that require multiple, repeated injection to an eye, e.g., increased risk of inflammation, infection, elevated intraocular pressure, and/or other adverse effects.

In some embodiments, MW-opsin gene therapy or a pharmaceutical composition thereof can be administered as a single dose or a one-time dose. In some embodiments, more than one administration may be employed to achieve the desired level of gene expression over a sustained period of various intervals, e.g., not more than once in at least 2 years, or at least 3, 4, 5, 6, 7, 8, 9, 10, or more years. In some embodiments, intravitreal injection of MW-opsin gene therapy obviates a patient's need to receive an approved protein injection for at least 1 year or 2, 3, 4, 5, 6, 7, 8, 9, 10, 20, 30 or more years.

With delivery of a MW-opsin transgene in vivo gene therapy, one would be able to administer the pharmaceutical composition comprising a polynucleotide sequence that encodes MW-opsin, in a single dose or a one-time dose. In some embodiments, the total number of doses of a gene therapy administered to a subject is not more than once in at least 1.5 years, in at least 2 years, at least 3 years, at least 4 years, at least 5 years, at least 6 years, at least 7 years, at least 8 years, at least 9 years, or at least 10 years. In some embodiments, administration of a gene therapy comprising a polynucleotide sequence encoding MW-opsin is only one time or once in the life of a patient. In some embodiments, one-time administration of a gene therapy comprising a polynucleotide sequence encoding MW-opsin can produce a therapeutic effect in a patient that lasts for more than 1 year, for more than 2, 3, 4, 5, 6, 7, 8, 9, 10, 15, 20, 25, 30, 35, 40, 45, 50 or more years. In some embodiments, a gene therapy comprising a polynucleotide sequence encoding MW-opsin is administered not more than once to a patient in at least 2 or more, at least 3 or more, at least 4 or more, at least 5 or more, at least 6 or more, at least 7 or more, at least 8 or more, at least 9 or more, or at least 10 or more years. In some embodiments, a gene therapy comprising a polynucleotide sequence encoding MW-opsin is administered to a patient who was initially responsive to at least one current standard of care or at least one existing therapy, e.g., anti-VEGFT agent. In some embodiments, the gene therapy is administered to patients who received a pre-treatment with anti-VEGF therapy before receiving the gene therapy.

In some embodiments, the one-time administration of a gene therapy comprising a polynucleotide sequence encoding MW-opsin obviates the need for the patient to receive an anti-VEGF agent or any other protein-based therapeutics or standard of care treatments in the eye for more than a year, for more than 1.5 years, or for more than 2, 3, 4, 5, 6, 7, 8, 9, 10, 15, 20, 25, 30, 35, 40, 45, 50 or more years. In some embodiments, a patient who receives an injection of a gene therapy comprising a polynucleotide sequence encoding MW-opsin does not need any additional injections of an anti-VEGF agent or any other protein-based therapeutics or standard of care treatments in the eye for the remainder of the patient's life. In other embodiments, a patient who receives a one-time injection of MW-opsin gene therapy can commence therapy after anti-VEGF agent therapy and/or any other approved therapeutics, as needed, after at least 1.5, 2, 5, 10, 15, 20, 25, 30, 35, 40, 45, 50 or more years have lapsed after receiving the gene therapy.

The present disclosure provides a method of enhancing or restoring visual function in an individual, the method comprising administering a pharmaceutical composition comprising a recombinant viral vector of the present disclosure (or a viral particle comprising the recombinant viral vector) to the eye of an individual. Following administration of the recombinant viral vector (or viral particle comprising the recombinant viral vector), the MW-opsin is produced in the retinal cell. Production of the MW-opsin in the retinal cell provides for enhanced or restored visual function in the individual.

Expression of an MW-opsin polypeptide in a retinal cell in an individual provides for patterned vision and image recognition by the individual. Image recognition can be of a static image and/or of a moving image.

Expression of an MW-opsin polypeptide in a retinal cell in an individual provides for image recognition at a light intensity of from about 10⁻⁴ W/cm² to about 10 W/cm². For example, in some cases, expression of an MW-opsin polypeptide in a retinal cell in an individual provides for image recognition at a light intensity of from about 10⁻² W/cm² to about 10⁻⁴ W/cm², from about 10⁻⁴ W/cm² to about 1 W/cm², from about 10⁻⁴ W/cm² to about 10⁻¹ W/cm², or from about 10⁻⁴ W/cm² to about 5×10⁻¹ W/cm². In some cases, expression of an MW-opsin polypeptide in a retinal cell in an individual provides for image recognition at a light intensity of from about 10⁻⁴ W/cm² to about 10⁻³ W/cm², from about 10⁻³ W/cm² to about 10⁻² W/cm², from about 10⁻² W/cm² to about 10⁻¹ W/cm², or from about 10⁻¹ W/cm² to about 1 W/cm². In some cases, expression of an MW-opsin polypeptide in a retinal cell in an individual provides for image recognition at a light intensity of up to 2 W/cm² up to 3 W/cm², up to 4 W/cm², up to 5 W/cm², or up to 10 W/cm². Expression of an MW-opsin polypeptide in a retinal cell in an individual provides for image recognition at a light intensity of less than 5 W/cm², less than 4 W/cm², less than 3 W/cm², or less than 2 W/cm².

Expression of an MW-opsin polypeptide in a retinal cell in an individual provides for image recognition by the individual at a light intensity that is at least 10-fold lower than the light intensity required to provide for image recognition by an individual expressing a channelrhodopsin polypeptide in a retinal cell. For example, expression of an MW-opsin polypeptide in a retinal cell in an individual provides for image recognition by the individual at a light intensity that is at least 10-fold lower, at least 25-fold lower, at least 50-fold lower, at least 100-fold lower, at least 150-fold lower, at least 200-fold lower, at least 300-fold lower, at least 400-fold lower, or at least 500-fold lower, than the light intensity required to provide for image recognition by an individual expressing a channel rbodopsin polypeptide in a retinal cell.

Expression of an MW-opsin polypeptide in a retinal cell provides for kinetics that are at least 2-fold faster than the kinetics conferred on a retinal cell by a rhodopsin polypeptide. For example, expression of an MW-opsin polypeptide in a retinal cell provides for kinetics that are at least 2-fold, at least 5-fold, at least 10-fold, at least 15-fold, at least 20-fold, at least 25-fold, at least 30-fold, at least 50-fold, at least 100-fold, or more than 100-fold, faster than the kinetics conferred on a retinal cell by a rhodopsin polypeptide.

A recombinant expression vector comprising a polynucleotide sequence encoding an MW-opsin polypeptide is administered in an amount effective to increase visual function in an individual by at least 10%, at least 15%, at least 20%, at least 25%, at least 30%, at least 40%, at least 50%, at least 2-fold, at least 5-fold, at least 10-fold, or more than 10-fold, compared with the visual function before administration of the recombinant expression vector. Tests for visual function are known in the art, and any known test can be applied to assess visual function.

The present disclosure provides compositions comprising one or more recombinant expression vectors comprising a polynucleotide sequence encoding MW-opsin. When the composition is administered to an individual in need thereof, the polynucleotide sequence encoding the MW-opsin polypeptide is expressed in an eye of a subject in need thereof, such that the MW-opsin is produced in the eye of the subject, one or more beneficial clinical outcomes results. For example, when the composition is administered to an eye of an individual in need thereof, the one or more nucleotide sequences encoding the MW-opsin is expressed in an eye of a subject in need thereof, such that the MW-opsin is produced in the eye of the subject, one or more beneficial clinical outcomes results. When the polynucleotide sequence encoding the MW-opsin is expressed in an eye of a subject in need thereof, such that the one or more cone opsins are produced in the eye of the subject, one or more beneficial clinical outcomes results.

Beneficial clinical outcomes include: 1) the subject can distinguish between an image comprising a vertical line and an image comprising a horizontal line in a spatial pattern discrimination assay; 2) the subject can distinguish between an image comprising a static line and an image comprising a moving line in a spatial pattern discrimination assay; 3) the subject can distinguish between flashing light and constant light in a temporal light pattern assay; 4) the subject can recognize an image at a light intensity of from about 104 W/cm² to about 10 W/cm in an image recognition assay; and 5) subject can distinguish between an area with white light and an area without white light in a light avoidance assay.

Whether a composition provides one or more of the above-noted beneficial clinical outcomes can be determined using tests that are known in the art. See e.g., Leinonen and Tanila (2017) Behavioural Brain Research pii: S0166-4328(17)30870-7; Caporale et al. (2011) Molecular Therapy 19, 1212-9; Gaub et al. (2014) Proc. Natl. Acad. Sci. USA 111, E5574-83; Gaub et al. (2015) Molecular Therapy 23:1562; and Berr et al. (2017) Nat. Commun. 8:1862.

The present disclosure composition comprising recombinant expression vectors comprising a polynucleotide sequence encoding MW-opsin, wherein, when: i) the composition is administered to an individual in need thereof; or ii) the composition is administered to an eye of an individual in need thereof, such that the polynucleotide sequence is expressed in an eye of a subject in need thereof (such that the MW-opsin is produced in the eye of the subject), the subject can distinguish between an image comprising a vertical line and an image comprising a horizontal line in a spatial pattern discrimination assay. The present disclosure composition comprising recombinant expression vectors comprising a polynucleotide sequence encoding MW-opsin, wherein, when the polynucleotide sequence is expressed in an eye of a subject in need thereof (such that the MW-opsin is produced in the eye of the subject), the subject can distinguish between an image comprising a vertical line and an image comprising a horizontal line in a spatial pattern discrimination assay.

The present disclosure composition comprising recombinant expression vectors comprising a polynucleotide sequence encoding MW-opsin, wherein, when: i) the composition is administered to an individual in need thereof; or ii) the composition is administered to an eye of an individual in need thereof, such that the polynucleotide sequence is expressed in an eye of a subject in need thereof (such that the MW-opsin is produced in the eye of the subject), the subject can distinguish between an image comprising a static line and an image comprising a moving line in a spatial pattern discrimination assay. The present disclosure provides a composition comprising recombinant expression vectors comprising a polynucleotide sequence encoding MW-opsin, wherein, when said polynucleotide sequence is expressed in an eye of a subject in need thereof (such that the MW-opsin is produced in the eye of the subject), the subject can distinguish between an image comprising a static line and an image comprising a moving line in a spatial pattern discrimination assay.

The present disclosure composition comprising recombinant expression vectors comprising a polynucleotide sequence encoding M-W-opsin, wherein, when: i) the composition is administered to an individual in need thereof: or ii) the composition is administered to an eye of an individual in need thereof, such that the polynucleotide sequence is expressed in an eye of a subject in need thereof (such that the MW-opsin is produced in the eye of the subject), the subject can distinguish between flashing light and constant light in a temporal light pattern assay. The present disclosure provides a composition comprising recombinant expression vectors comprising the polynucleotide sequence encoding the MW-opsin, wherein, when said polynucleotide sequence is expressed in an eye of a subject in need thereof (such that the MW-opsin is produced in the eye of the subject), the subject can distinguish between flashing light and constant light in a temporal light pattern assay.

The present disclosure composition comprising recombinant expression vectors comprising a polynucleotide sequence encoding MW-opsin, wherein, when: i) the composition is administered to an individual in need thereof; or ii) the composition is administered to an eye of an individual in need thereof, such that the polynucleotide sequence is expressed in an eye of a subject in need thereof (such that the MW-opsin is produced in the eye of the subject), the subject can recognize an image at a light intensity of from about 104 W/cm² to about 10 V/cm² in an image recognition assay. The present disclosure provides a composition comprising recombinant expression vectors comprising a polynucleotide sequence encoding MW-opsin, wherein, when said the polynucleotide sequence is expressed in an eye of a subject in need thereof (such that the MW-opsin is produced in the eye of the subject), the subject can recognize an image at a light intensity of from about 10⁻⁴ W/cm² to about 10 W/cm² (e.g., a light intensity of from about 10⁻⁴ W/cm² to about 10⁻³ W/cm², from about 10⁻³ W/cm² to about 10⁻² W/cm², from about 10⁻² W/cm² to about 10⁻¹ W/cm², or from about 10⁻¹ W/cm² to about 1 W/cm². In some cases, expression of an MW-opsin polypeptide in a retinal cell in an individual provides for image recognition at a light intensity of up to 2 W/cm² up to 3 W/cm², up to 4 W/cm², up to 5 W/cm², or up to 10 W/cm²) in an image recognition assay.

The present disclosure composition comprising recombinant expression vectors comprising a polynucleotide sequence encoding MW-opsin, wherein, when: i) the composition is administered to an individual in need thereof: or ii) the composition is administered to an eye of an individual in need thereof, such that the MW-opsin is expressed in an eye of a subject in need thereof (such that the MW-opsin is produced in the eye of the subject), the subject can distinguish between an area with white light and an area without white light in a light avoidance assay. The present disclosure provides a composition comprising recombinant expression vectors comprising a polynucleotide sequence encoding MW-opsin, wherein, when the polynucleotide sequence is expressed in an eye of a subject in need thereof (such that the MW-opsin is produced in the eye of the subject), the subject can distinguish between an area with white light and an area without white light in a light avoidance assay.

In another aspect, the present disclosure provides the recombinant expression vector as described above or the pharmaceutical composition as described above for use in treating a subject in need thereof. In one embodiment, the recombinant expression vector as described above or the pharmaceutical composition as described above restores or enhances visual function in a subject.

### Kits for Therapeutic Use

The present disclosure also provides kits for use of the compositions described herein. For example, the present disclosure provides kits comprising a gene therapy system as described herein; a viral particle or set of viral particles comprising a gene therapy system as described herein; and/or a polynucleotide or set of polynucleotides comprising a gene therapy system as described herein.

In some embodiments, the kit can additionally comprise instructions for use in any of the methods described herein. The included instructions may comprise a description of: (i) the delivery of a gene therapy system as described herein; a viral particle or set of viral particles comprising a gene therapy system as described herein; and/or a polynucleotide or set of polynucleotides comprising a gene therapy system as described herein.

The kit may further comprise a description of selecting a subject suitable for treatment based on identifying whether the subject is in need of the treatment. The instructions may include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an injector. A kit may have a sterile access port (for example, a vial having a stopper pierceable by a hypodermic injection needle). In some embodiments, optionally, the kit further comprises a device for administration, such as a syringe, filter needle, extension tubing, cannula, or subretinal injector.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Polynucleotide Hybridization (B. D. Hames & S. J. Higgins eds. (1985; Transcription and Translation (B. D. Hames & S. J. Higgins, eds. (1984; Animal Cell Culture (R. I. Freshney, ed. (1986; Immobilized Cells and Enzymes (IRL Press, (1986; and B. Perbal, A practical Guide To Molecular Cloning (1984); F. M. Ausubel et al. (eds.).

### Pharmaceutical Compositions

The pharmaceutical compositions according to the present disclosure are formulated according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

In some embodiments, the composition can further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more preferably, the poly-L-glutamate is present in the composition of the present disclosure at a concentration less than 6 mg/ml. The transfection facilitating agent can also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid can also be used administered in conjunction with the genetic construct. In some embodiments, the pharmaceutical composition comprising the rAAV virion and/or recombinant expression vector encoding the MW-opsin polypeptide can also include a transfection facilitating agent such as lipids, liposomes, including lecithin liposomes or other liposomes known in the art, as a DNA-liposome mixture (see, e.g., WO 9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid. The compositions of the disclosure can optionally comprise medicinal agents, pharmaceutical agents, carriers, adjuvants, dispersing agents, diluents, and the like.

In some embodiments, the polynucleotide constructs of the disclosure can be formulated for administration in a pharmaceutical carrier in accordance with known techniques. See, e.g., Remington, The Science and Practice of Pharmacy (21th Ed. 2005). In the manufacture of a pharmaceutical composition, the rAAV virions and/or polynucleotide constructs are typically admixed with, inter alia, an acceptable carrier. The carrier can be a solid (including a powder) or a liquid, or both, and is preferably formulated with the compound as a unit-dose composition, for example, which can contain from 0.01 or 0.5% to 95% or 99% by weight of the compound. One or more compounds can be incorporated in the compositions of the disclosure, which can be prepared by any of the well-known techniques of pharmacy.

A carrier can be a solvent or dispersion medium containing, for example, water, saline, ethanol, a polyol (for example, glycerol, propylene glycol, and liquid polyethylene glycol, and the like), and any combination thereof. The proper fluidity can be maintained, for example, by the use of a coating such as lecithin, by the maintenance of the required particle size in the case of dispersion and by the use of surfactants such as polysorbates (e.g., Tween™, polysorbate 20, polysorbate 80), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol (Triton X100™), N,N-dimethyldodecylamine-N-oxide, hexadecyltrimethylammonium bromide (HTAB), polyoxyl 10 lauryl ether, Brij 721™, bile salts (sodium, deoxycholate, sodium cholate), pluronic acids (F-68, F-127), polyoxyl castor oil (Cremophor™) nonylphenol ethoxylate (Tergitol™), cyclodextrins and, ethylbenzethonium chloride (Hyamine™) Prevention of the action of microorganisms can be achieved by various antibacterial and antifungal agents, for example, parabens, chlorobutanol, phenol, cresol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin. In some embodiments, the pharmaceutical carrier includes sodium phosphate, sodium chloride, polysorbate, and sucrose. In some embodiments, a pharmaceutical composition comprises a surfactant, e.g., non-ionic surfactant such as polysorbate, poloxamer, or pluronic. In some embodiments, the addition of a non-ionic surfactant reduces aggregation in a suspension or solution. For intravitreal administration, suitable carriers include physiological saline, bacteriostatic water, phosphate buffered saline (PBS), and/or an isotonic agent, e.g., glycerol.

In all cases, the pharmaceutical composition must be sterile and should be fluid to the extent that easy syringability or injectability exists. It must be stable under the conditions of manufacture and storage and must be preserved against the contaminating action of microorganisms such as bacteria and fungi. In some embodiments, the pharmaceutical composition can include an isotonic agent, such as a salt or glycerol. In some embodiments, a surfactant or a stabilizer is added to the pharmaceutical composition to prevent aggregation. Additionally, cryoprotectants, such as alcohols, DMSO, glycerol, and PEG can be used as a stabilizer under the freezing or drying conditions of lyophilization, or be used as a stabilizer for making a refrigerated suspension.

In one embodiment, the pharmaceutical composition comprises the rAAV virions disclosed herein, one or more pharmaceutically acceptable salts at a molar concentration of from about 10 mM to about 200 mM. In some embodiments, the one or more pharmaceutically acceptable salts in the pharmaceutical composition are at a concentration of about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, or more than 200 mM. In one embodiment, the pharmaceutical composition comprises the rAAV virions disclosed herein, one or more polymers at a concentration of from about 0.0001% to about 0.01%. In some embodiments, the polymer(s) in the pharmaceutical composition are at a concentration of about 0.0001%, about 0.0002%, about 0.0003%, about 0.0004%, about 0.0005%, about 0.0006%, about 0.0007%, about 0.0008%, about 0.0009%, about 0.001%, about 0.002%, 0.003%, 0.004%, about 0.005%, about 0.006%, about 0.007%, about 0.008%, about 0.009%, or about 0.01%. In one embodiment, the pharmaceutical composition has a pH of from about 4.5 to about 7.5. In some embodiments, the pH of the pharmaceutical composition has a pH of about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In one embodiment, the pharmaceutical composition comprises the rAAV virions disclosed herein, 10 mM sodium phosphate, 180 mM sodium chloride, 0.005% poloxamer 188 and has a pH of 7.3.

In some embodiments, a lower amount or range of vector genomes is selected for a unit dose to avoid aggregation. In some embodiments, a higher amount or range of vector genomes is selected for a unit dose so that a smaller volume can be used for injection. Smaller volume (e.g., less than 50, 40, 30, 20, 10, or 5 μL) of injection can help to reduce changes in ocular pressure and other adverse effects associated with intravitreal injection. In some embodiments, a higher concentration of rAAV also helps to ensure efficient delivery of the therapeutic transgene into target cells.

In some embodiments, pharmaceutical compositions disclosed herein are designed, engineered, or adapted for administration to a primate (e.g., non-human primate and human subjects) via intraocular, intravitreal, or subretinal injection. In some embodiments, a pharmaceutical composition comprising rAAV virions comprising a polynucleotide sequence that encodes a MW-opsin polypeptide is formulated for intravitreal injection into an eye of a subject. In some embodiments, the pharmaceutical composition is formulated to a concentration that allows intravitreal injection of a volume not more than about 2, 2.5, 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL. In some embodiments, methods of treatment disclosed herein comprises intravitreal injection of a volume of about 2, 5, 10, 15, 20, 25, 30, 35, 40, 45, 50, 55, 60, 65, 70, 75, 80, 85, 90, 95, 100, 110, 120, 130, 140, 150 μL of a solution or suspension comprising a rAAV comprising a polynucleotide sequence that encodes a MW-opsin polypeptide as disclosed herein.

As discussed above, a further aspect of the disclosure is a method of treating subjects in vivo, comprising administering to a subject a pharmaceutical composition comprising polynucleotide constructs of the disclosure in a pharmaceutically acceptable carrier, wherein the pharmaceutical composition is administered in a therapeutically effective amount. Administration of the compounds of the present disclosure to a eukaryotic subject in need thereof can be by any means known in the art for administering compounds. Another aspect of the invention provides a pharmaceutical composition comprising the recombinant expression vector described above and a pharmaceutically acceptable excipient. In an embodiment, the pharmaceutically acceptable excipient comprises saline. In a further embodiment, the composition is sterile.

In another aspect, the present disclosure provides the recombinant expression vector as described above or the pharmaceutical composition as described above for use in the manufacture of a medicament.

Compositions of the present disclosure suitable for intraocular, intravitreous, or subretinal administration comprise sterile aqueous and non-aqueous injection solutions of the rAAV virions and/or polynucleotide constructs described herein, which preparations are preferably isotonic with the vitreous of the intended recipient. These preparations can contain anti-oxidants, buffers, bacteriostats and solutes, which render the composition isotonic with the vitreous of the intended recipient. Aqueous and non-aqueous sterile suspensions can include suspending agents and thickening agents. The compositions can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present disclosure, there is provided an injectable, stable, sterile composition comprising a polynucleotide or rAAV of the disclosure, in a unit dosage form in a sealed container. In some embodiments, a lyophilized form of the pharmaceutical composition is provided in a kit with a solution or buffer for reconstituting the pharmaceutical composition before administration. In some embodiments, the pharmaceutical compositions disclosed herein are supplied as a solution, a homogeneous solution, a suspension, or a refrigerated suspension. In some embodiments, a pharmaceutically acceptable excipient comprises a surfactant that prevents aggregation in the pharmaceutical composition disclosed herein. In some embodiments, the refrigerated suspension is provided in a kit, which can include a syringe and/or buffer for dilution. In some embodiments, the refrigerated suspension is provided as a pre-filled syringe. In some embodiments, method of treatment or prevention of an eye disease or condition as disclosed herein comprises warming the refrigerated suspension to room temperature and/or agitating the suspension to ensure even distribution before administering or intravitreal injection to a patient. In some embodiments, the suspension is diluted before administering to a patient.

In some embodiments, the lyophilized or a suspension of the pharmaceutical composition comprising a MW-opsin gene therapy as disclosed herein has a volume (or reconstituted volume) of about 10, 20, 30, 40, 50, 60, 70, 80, 90, 100, 200, 300, 400, 500, 600, 700, 800, 900, or 1000 μL. In some embodiments, the lyophilized or suspension form of the pharmaceutical composition comprising the MW-opsin gene therapy as disclosed herein has a volume of between 0.1 to 0.5 mL, between 0.1 to 0.2 mL, between 0.3 to 0.5 mL, between 0.5-1.0 mL, between 0.5-0.7 mL, between 0.6 to 0.8 mL, between 0.8 to 1 mL, between 0.9 to 1.1 mL, between 1.0 to 1.2, or between 1.0 to 1.5 mL. In other embodiments, the reconstituted volume is no more than about 0.1, 0.2, 0.3, 0.4, 0.5, 0.6, 0.7, 0.8, 0.9, 1.0, 1.1, 1.2, 1.3, 1.4, or 1.5 mL. In some embodiments, the reconstituted solution or suspension is filtered before administration. In some embodiments, a filter or a filter syringe is used for filtering the pharmaceutical composition before administration to a patient.

Further, the present disclosure provides liposomal formulations of the polynucleotide constructs of the disclosure disclosed herein. The technology for forming liposomal suspensions is well known in the art. As aqueous-soluble material, using conventional liposome technology, the polynucleotide constructs of the disclosure can be incorporated into lipid vesicles. In such an instance, due to the water solubility of the compound, the compound will be substantially entrained within the hydrophilic center or core of the liposomes. The lipid layer employed can be of any conventional composition and can either contain cholesterol or can be cholesterol-free. The liposomes, which are produced, can be reduced in size through the use of, for example, standard sonication and homogenization techniques. The liposomal compositions containing the compound disclosed herein can be lyophilized to produce a lyophilizate, which can be reconstituted with a pharmaceutically acceptable carrier, such as water, to regenerate a liposomal suspension.

In some embodiments, the pharmaceutical composition comprising the polynucleotide constructs of the disclosure can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. In some embodiments, hydrochloric acid and sodium hydroxide are used to adjust the pH of the solution. In some embodiments, the suspension is at a neutral pH, or at a pH between 6.5 to 7.5. In some embodiments, the pH of the suspension is slightly basic (e.g., pH about 7.5, 8, 8.2, 8.4, 8.5, or 9). In some embodiments, the pH of the suspension or solution is slightly acidic (e.g., pH about 6.5, 6.3, 6.1, 6, 5.5, or 5). In some embodiments, the suspension is refrigerated as a solution. In some embodiments, the suspension comprises refrigerated micelles. In some embodiments, the suspension is refrigerated and agitated before administration.

Further, the compositions can contain microbial preservatives. Useful microbial preservatives include methylparaben, propylparaben, and benzyl alcohol. Other additives that are well known in the art include, e.g., detackifiers, anti-foaming agents, antioxidants (e.g., ascorbyl palmitate, butyl hydroxy anisole (BHA), butyl hydroxy toluene (BHT) and tocopherols, e.g., α-tocopherol (vitamin E)), preservatives, chelating agents (e.g., EDTA and/or EGTA), viscomodulators, tonicifiers (e.g., a sugar such as sucrose, lactose, and/or mannitol), colorants, odorants, opacifiers, suspending agents, binders, fillers, plasticizers, lubricants, and mixtures thereof. The amounts of such additives can be readily determined by one skilled in the art, according to the particular properties desired.

In some embodiments, any suitable method can be used in the biochemical purification of recombinant viruses (e.g., rAAV) for use in a pharmaceutical composition as described herein. Recombinant AAV viruses can be harvested directly from cells, or from the culture media comprising cells. Virus can be purified using various biochemical means, such as gel filtration, filtration, chromatography, affinity purification, gradient ultracentrifugation, or size exclusion methods before lyophilizing or making a suspension of the rAAV viruses.

In some embodiments, a pharmaceutical composition disclosed herein is adapted for gene therapy or for intravitreal delivery of a MW-opsin polypeptide as the therapeutic agent in human patients or non-human primates. In some embodiments, a unit dose of the pharmaceutical composition comprises between 1×10¹⁰ to 1×10¹³ viral genomes (vg). In some embodiments, a unit dose comprises about 2.1×10¹¹, about 2.1×10¹², or about 2.1×10¹³ vector genome. In some embodiments, the unit dose of the pharmaceutical composition of the disclosure is 1×10¹⁰ to 3×10¹² vector genomes. In some cases, the unit dose of the pharmaceutical composition of the disclosure is 1×10⁹ to 3×10¹³ vector genomes. In some cases, the unit dose of the pharmaceutical composition of the disclosure is 1×10¹⁰ to 1×10¹¹ vector genomes. In some cases, the unit dose of the pharmaceutical composition of the disclosure is 1×10⁸ to 3×10¹⁴ vector genomes. In some cases, the unit dose of the pharmaceutical composition of the disclosure is at least 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷ and 1×10¹⁸ vector genomes. In some cases, the unit dose of the pharmaceutical composition of the disclosure is 1×10¹⁰ to 5×10¹³ vector genomes. In some cases, the unit dose of the pharmaceutical composition of the disclosure is at most about 1×10⁸, 1×10⁹, 1×10¹⁰, 1×10¹¹, 1×10¹², 1×10¹³, 1×10¹⁴, 1×10¹⁵, 1×10¹⁶, 1×10¹⁷ and 1×10¹⁸ vector genomes.

In some embodiments, the unit dose of rAAV of this disclosure is between 2×10¹¹ to 8×10¹¹ or between 2×10¹² to 8×10¹² vector genomes. In some embodiments, the unit dose of rAAV of this disclosure is between 10¹⁰ to 10¹³, between 10¹⁰ to 10¹¹, between 10¹¹ to 10¹², between 10¹² to 10¹³, or between 10¹³ to 10¹⁴ vector genomes.

In some cases, a composition comprising a recombinant viral expression vector comprising a polynucleotide sequence encoding an MW-opsin polypeptide is present in a buffered saline solution in an amount of from about 10⁸ to about 10¹⁵ viral genomes (vg) in a volume of from about 50 μL to about 100 μL, wherein the composition further comprises sodium phosphate, poloxamer 188 and has a pH of 7.3.

In particular embodiments, the polynucleotide constructs of the disclosure can be administered to the subject in a therapeutically effective amount, as that term is defined above. Dosages of pharmaceutically active compounds can be determined by methods known in the art, see, e.g., Remington, The Science And Practice of Pharmacy (21th Ed. 2005). The therapeutically effective dosage of any specific compound will vary somewhat for a given disclosed polynucleotide construct, and patient to patient, and will depend upon the condition of the patient and the route of delivery.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### Evaluation of Recombinant Expression Vector-Mediated Expression of Codon-Optimized Human MW-Opsin mRNA

A 6-well plate was seeded with 2.0×E+6 cells per well with 293T cells (passage <30) in DMEM+10% FBS. 18 h to 24 h later, each well was transfected with 2 μg of DNA or PEI-Max only (control). 3 days post-transfection the cells and media were recovered, cells were pelleted by centrifugation and stored at −80° C. until ready to process. For each sample, RNA was extracted with an RNeasy Plus kit following the manufacturer's instructions, eluted in 20 μL of water and RNA concentration was determined with Nanodrop. 2 μg of total RNA was used to generate cDNA with the High Capacity cDNA Reverse Transcription Kit with RNAse inhibitor, according to the manufacturer's instructions. For qPCR, each sample (10 ng) was run in triplicate with each MWO primers/probe pair provided below:

TABLE 1

| Description | Source | Product Details |
|---|---|---|
| Human MW-opsin | IDT | For: GTCTGAATCCACCCAGAAGG |
| primers/probe | | Rev: TGCGAAGAAGGCGTATGG |
| (hMWO) | | Probe: TGATGGTCCTGGCATTCTG |
| | | CTTCT |
| Codon-optimized | IDT | For: TCACTTACACCAACTCCAACTC |
| human MW-opsin | | Rev: GAAGATCATCCACACGCTAGTC |
| primers/probe | | Probe: TTATCACATTGCGCCGAGAT |
| pair 1 | | GGGT |
| (cohMWO pair #l) | | |
| Codon-optimized | IDT | For: GTCCGCGACCATCTACAATC |
| human MW-opsin | | Rev: ATCCGTCGTCCACTTTCTTTC |
| primers/probe | | Probe: CGAAGAGTTGCAGGATGCA |
| pair 2 | | GTTGC |
| (cohMWO pair #2) | | |

The exemplary recombinant expression vectors being tested had a first ITR sequence of SEQ ID NO: 1, a promoter sequence of SEQ TD NO: 2, a MW-opsin transgene sequence of SEQ ID NO: 3, an enhancer sequence of SEQ TD NO: 4, a polyA/termination sequence of SEQ ID NO: 5, an intron sequence of SEQ ID NO: 6, and a second ITR sequence of SEQ ID NO: 7, in the 5′ to 3′ orientation. The exemplary recombinant expression vector includes a first AAV2 ITR sequence, a CAG promoter sequence, a human MW-opsin transgene sequence, a WPRE sequence, an intron sequence derived from the human MW-opsin gene sequence, a polyA/termination sequence, a second AAV2 ITR sequence, in the 5′ to 3′ orientation. In some experiments, the recombinant expression vector further comprised a polynucleotide sequence conferring resistance to ampicillin (REV_Amp). In some experiments, the recombinant expression vector further comprised a polynucleotide sequence conferring resistance to kanamycin (REV_Kan). Following qPCR, the following average Ct values were reported:

TABLE 2

| | Average Ct values | | | |
|---|---|---|---|---|
| | | | | GAPDH |
| Sample Name | hMWO | cohMWO pair#1 | cohMWO pair#2 | (Control) |
| PEI alone | 31.10 | 31.10 | 31.10 | 17.08 |
| REV_Amp | 29.65 | 12.25 | 12.22 | 17.85 |
| REV_Kan | 29.37 | 12.19 | 12.16 | 17.65 |

The Ct (cycle threshold) is defined as the number of qPCR cycles required for the fluorescent signal to cross the threshold, i.e., exceeds background level. Here, the values of 12.25 and 12.22 indicate that codon-optimized human MW-opsin mRNA was detected in 293T cells that were transfected with the REV_Amp vector. Likewise, the values of 12.19 and 12.16 indicate that codon-optimized human MW-opsin mRNA was detected in 293T cells that were transfected with REV_Kan vector. The REV_Kan and REV_Amp vectors with the codon-optimized human MW-opsin sequence demonstrated better mRNA expression in transfected 293T cells as compared to a similar vector that relied on a mini-CAG promoter and a non-codon-optimized sequence encoding human MW-opsin (average Ct values of 17.78 with hMWO, average Ct values of 30.22 with cohMWO pair #1, and average Ct values of 30.10 with cohMWO pair #2).

### Example 2

### Evaluation of Recombinant Expression Vector-Mediated Expression of Codon-Optimized Human MW-Opsin Protein

A 24-well plate was seeded with 250,000 cells per well with 293T cells (passage <30) in DMEM+10% FBS. 18 h to 24 h later, each well was transfected with 2 μg of DNA or PEI-Max only (control). 3 days post-transfection, the media was carefully removed from the well and replaced with a fresh solution of 4% paraformaldehyde (4% PFA, 1 mL/well) for 10 minutes with gentle rotation. 4% PFA was removed and each well was washed 3 times with PBS for 5 minutes each. Cells were permeabilized in Blocking Buffer (1% BSA, 0.1% Triton-X100) for 30 min at room temperature, with gentle rotation. At the end of the incubation, Blocking Buffer was replaced with the primary antibody, diluted 1:200 in 1% BSA, 0.1% TritonX-100. Incubation with the primary antibody (Red/Green opsin, rabbit polyclonal antibody, Millipore Sigma AB5405) was allowed to proceed overnight at 4° C. with gentle rotation, in the dark. The next morning cells were rinsed with PBS, 3×5 minutes before starting the incubation with the secondary antibody (1:10,000 dilution in 1% BSA, 0.1% Triton-X100) for 2 hours at room temperature, in the dark. After 3 more rinses with PBS (3×5 minutes) the coverslips were mounted on slides with DAPI-containing mounting media and sealed with clear nail polish. Slides were imaged immediately then stored at 4° C. in the dark.

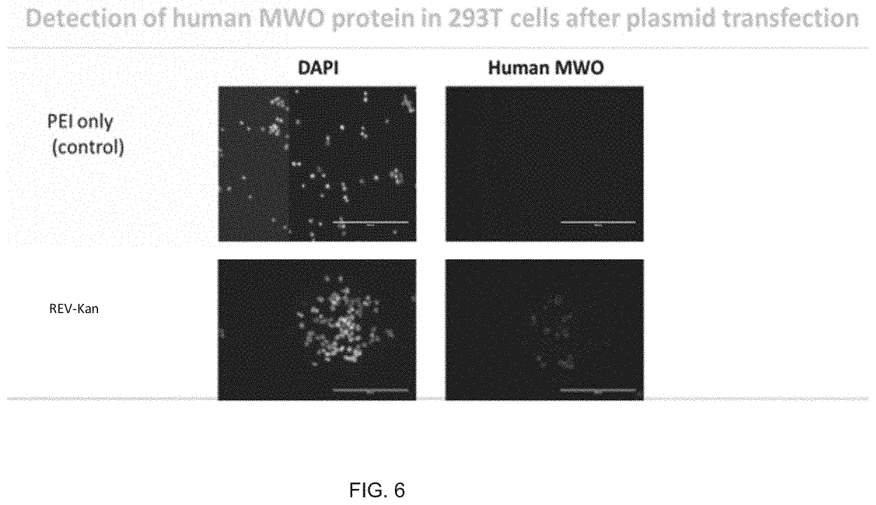

The exemplary recombinant expression vector being tested had a first ITR sequence of SEQ ID NO: 1, a promoter sequence of SEQ ID NO: 2, a MW-opsin transgene sequence of SEQ ID NO: 3, an enhancer sequence of SEQ ID NO: 4, a polyA/termination sequence of SEQ ID NO: 5, an intron sequence of SEQ ID NO: 6, and a second ITR sequence of SEQ ID NO: 7, in the 5′ to 3′ orientation. The recombinant expression vector further comprised a polynucleotide sequence conferring resistance to kanamycin (REV_Kan). The imaged results are illustrated in FIG. 6. Based on the fluorescence observed in the bottom right panel as compared to the top right panel where lower fluorescence was observed, codon-optimized human MW-opsin protein was expressed and detected by a rabbit anti-opsin polyclonal antibody.

### Example 3

### Evaluation of ITR Stability of Recombinant Expression Vector of Invention

The ITR polynucleotides of the present invention were further optimized to confer integrity and stability to the recombinant expression vectors of the present invention over recombinant expression vectors not having such ITR sequences. The following description explains the evaluation of the stability of ITRs in REV_Kan.

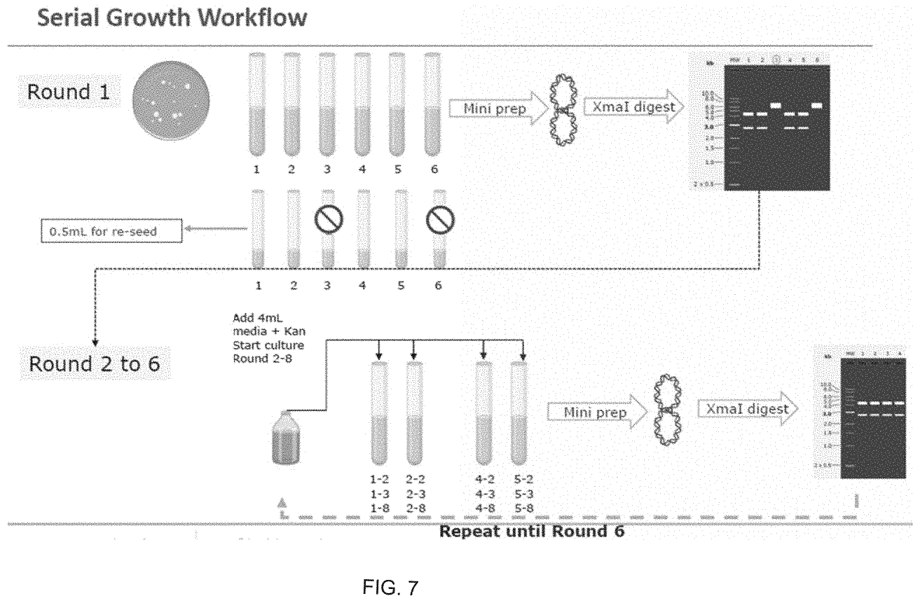

REV_Kan (1 ng) was transformed in NESbtl cells according to the manufacturer's high efficiency protocol. The bacterial cells were plated on an agar plate containing 50 mg/mL kanamycin and incubated overnight (12-16 h) at 37° C. Four colonies were picked and used to inoculate a 5 mL starter culture (LB+Kan 50 mg/mL). Each starter culture was incubated overnight (12-16 h) at 37° C. with 250 rpm shaking (Round 1). The next morning, 500 μL of each Round 1 starter culture was used to inoculate a new 5 mL starter culture (Round 2), and the remainder of the culture (4.5 mL) was pelleted and the DNA extracted from the cells using Qiagen's miniprep kit. This was repeated 4 times to complete 6 rounds of culture. See FIG. 7.

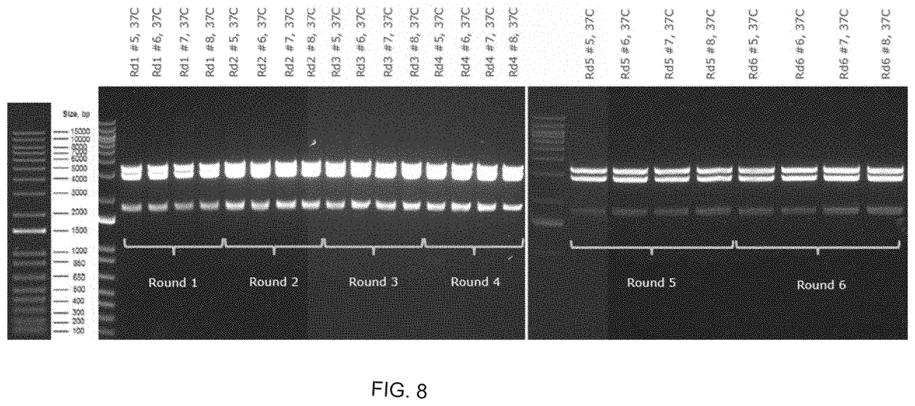

The DNA from all clones at each round was digested with XmaI enzyme for 2 hours at 37° C. Once the digest was complete, the samples were loaded on a 1% agarose gel and run from 45 min at 150V. The gel was imaged, and the banding pattern was examined. See FIG. 8. Where ITR sequences are intact after several rounds of culture, the expected DNA migration pattern includes 3257, 2798, 1734, 11, and 11 bp bands. If a deletion in the 5′ ITR occurred, the expected DNA migration pattern includes 6049, 1734, and 11 bp bands. If a deletion in the 3′ ITR occurred, the expected DNA migration pattern includes 4985, 2798, and 11 bp bands. The presence of bands at 6049 and 4985 bp indicates some recombination of the ITR has occurred.

After six rounds of culture (˜126 generations), no evidence of ITR recombination was detected for all clones, indicating that the ITRs of the recovered REV_Kan recombinant expression vector were stable.

### Example 4

Molecular Localization Studies of an Intravitreal Injection of Vector-Mediated Expression of Codon-Optimized Human MW-Cone Opsin in Cynomolgus Monkeys with a 13-Week Observation Period

The purpose of this molecular localization study was to determine ocular distribution of exemplary virion particles of the present invention, when administered via intravitreal (IVT) injection to cynomolgus macaques. Animals were injected once with 5×10¹⁰ or 4.5×10¹¹ vector genomes (vg) and observed for 4 weeks (interim sacrifice) and 13 weeks (terminal sacrifice). The exemplary recombinant expression vectors within the virion particles being tested had a first ITR sequence of SEQ ID NO: 1, a promoter sequence of SEQ ID NO: 2, a MW-opsin transgene sequence of SEQ ID NO: 3, an enhancer sequence of SEQ ID NO: 4, a polyA/termination sequence of SEQ ID NO: 5, an intron sequence of SEQ ID NO: 6, and a second ITR sequence of SEQ ID NO: 7, in the 5′ to 3′ orientation. The exemplary recombinant expression vectors include a first AAV2 ITR sequence, a CAG promoter sequence, a human MW-opsin transgene sequence, a WPRE sequence, an intron sequence derived from the human MW-opsin gene sequence, a polyA/termination sequence, a second AAV2 ITR sequence, in the 5′ to 3′ orientation. In situ hybridization for the vector sequence and immunostaining for human cone opsin and adeno associated virus (AAV) capsid proteins were performed on paraffin embedded eyes fixed in Modified Davidson's reagent from both control and exemplary vector-injected animals.

In situ hybridization detected the exemplary vector sequence in test injected eyes, but not in contralateral uninjected and in vehicle control animal eyes, of all interim and terminal necropsy animals. The exemplary nucleic acid signal was localized in multifocal areas of central retina (abundantly in macula), but more uniform distribution in peripheral retina. Signal was also present in ciliary body, iris, iridocorneal angle, lens capsule and optic nerve. In retina, the exemplary nucleic acid signal was abundant within retinal ganglion cells and nerve fiber layer, in moderate levels within inner plexiform layer, inner nuclear layer and occasionally within outer nuclear layer and photoreceptors. DNase and RNase pretreatment experiments demonstrated presence of transgene mRNA in retina. Dual in situ hybridization experiments showed localization of the exemplary nucleic acid signal in few Glutamate Metabotropic Receptor 6 (GRM6) positive bipolar cells. The exemplary nucleic acid signal was higher with high dose group (4.5×10¹¹ vg/eye) compared to low dose group (5×10¹⁰ vg/eye), but there was no significant differences between interim and terminal sacrifice animals in the same dose group.

Immunohistochemistry demonstrated the presence of AAV capsid proteins and human medium wave cone opsin confirming transduction of the exemplary vector and presence of transgene product, respectively in eye of vector-dosed animals.

In situ hybridization (ISH) was performed using Advanced Cell Diagnostics (ACD) (Hayward, Calif.) and Ventana Medical Systems (Tuczon, Ariz.) probes and reagents. Antisense and sense probes for transgene [codon optimized human Medium Wave-Cone Opsin (MW-Opsin)] were designed by ACD based on sequences in the exemplary vector. Positive PPIB and negative DAPB control probe sets were included to ensure mRNA quality and specificity, respectively.

The specificity of the exemplary vector sense and antisense probes was assessed with negative control and positive control cell lines that were transfected with plasmid carrying transgene-ChrimsonR-eGFP and MW-Opsin, respectively. The cell lines were processed to formalin fixed paraffin embedded (FFPE) blocks for localization experiments.

Antisense and sense ISH was performed on blocks of all study animals.

The hybridization method followed protocols established by ACD and Ventana systems using Ventana mRNA Red or Brown chromogens. Briefly, 5 m sections were baked at 60 degrees for 60 minutes and used for hybridization. The deparaffinization and rehydration protocol was performed using a Sakura Tissue-Tek DR5 stainer with the following steps: 3 times xylene for 5 minutes each; 2 times 100% alcohol for 2 minutes; air dried for 5 minutes. Off-line manual pretreatment in 1× retrieval buffer at 98 to 104 degrees C. for 15 minutes. Optimization was performed by first evaluating PPIB and DAPB hybridization signal and subsequently using the same conditions for all slides. Following pretreatment the slides were transferred to a Ventana Ultra autostainer to complete the ISH procedure including protease pretreatment; hybridization at 43 degrees C. for 2 hours followed by amplification; and detection with HRP and hematoxylin counter stain.

RNase and DNase pretreatment experiments were performed on a few animals to demonstrate the presence of the exemplary vector DNA and MW-Opsin mRNA. 5 m sections of paraffin embedded eye sections were deparaffinized and treated with hydrogen peroxidase for 10 min at room temperature, after which target retrieval was performed. Then, the slides were treated with RNase A (Qiagen catalog #19101) for 30 min at 37° C. or DNase I (Qiagen catalog #79254) for 10 min at 40° C. Slides were then processed as described above.

Dual labeling ISH experiments were performed on few animals to determine localization of the exemplary nuclei acid signal within bipolar cells of inner nuclear layer. The signal for the exemplary vector antisense and GRM6 (encodes the metabotropic glutamate receptor 6-mGluR6) probes were detected by two different chromogenic substrates-HRP-C1-Teal and AP-C2-Red, respectively.

A modified semiquantitative H-score (as described below) was adapted to score the vector nucleic acid localization in various regions of the eye.

Semiquantitative modified H-Score=(0×percentage of “Score 0” cells)+(1×percentage of “Score 1” cells)+(2×precentage of “Score 2” cells)+(3×percentage of “Score 3” cells). A score of “0” represents no signal. “1” for 1-3 dots per cell. “2” for 4-10 dots per cell, “3” for >10 dots per cell and less than 10% of cells with clusters, and “4” for >10 dots per cell or more than 10% of cells with clusters.

Immunohistochemistry (IHC) staining for opsin and AAV capsid proteins was performed on a Ventana Discovery XT autostainer using standard Ventana Discovery XT reagents (Ventana, Indianapolis, Ind.). The antibodies and concentration used for the assays were listed in Table 2. Slides were deparaffinized then submitted to heat-induced antigen retrieval by covering them with Cell Conditioning 1 (CC1/pH8) solution according to the standard Ventana retrieval protocol. Visualization was obtained by incubation with the appropriate Ventana Discovery OmniMap HRP reagent as indicated below followed by Ventana Discovery ChromoMap 3,3′-Diaminobenzidine (DAB). Counterstaining was performed using Ventana Hematoxylin and Ventana Bluing reagent for 4 minutes each. Slides were dehydrated, cleared and coverslipped with a synthetic mounting medium. Positive and negative control tissues were included in each run.

TABLE 3
In Situ Hybridization Probes

| Probe | Target | Vendor | Cat # | Comment |
|---|---|---|---|---|
| DapB | Dihydrodipicolinate | ACD | 312039 | Negative control |
| | reductase | | | probe (bacterial |
| | | | | gene) |
| Mfa-PPIB | Peptidylprolyl | ACD | 424149 | Positive control |
| | Isomerase B | | | house keeping |
| | | | | gene probe for |
| | | | | cynomolgus |
| | | | | macaque |
| Antisense | Human MW-Opsin | ACD | 1080809 | — |
| | mRNA and AAV | | | |
| | genome | | | |
| Sense | AAV genome | ACD | 1080879 | — |
| Mfa-GRM6 | Metabotropic | ACD | 1155679 | ON bipolar cell |
| | glutamate receptor 6 | | | marker |

TABLE 4
Immunohistochemistry Antibodies

| Target | Vendor | Catalog # | Comment |
|---|---|---|---|
| AAV VP1, VP2, | Progen | 65158 | — |
| VP3 | | | |
| Red/green opsin | Sigma | AB5405 | Polyclonal |
| | | | antibodies to |
| | | | medium and long |
| | | | wave opsin |

The specificity of the antisense and sense probes was assessed with known positive (transfected with MW-Opsin) and negative control cell pellets (transfected with ChrimsonR-eGFP). As expected, the positive control cells showed ample number of nucleic acid signal with antisense and sense probes, whereas the negative cells were negative for antisense and sense probes. ISH conditions were satisfactory as both the cell lines showed moderate levels of PPIB signal (endogenous control).

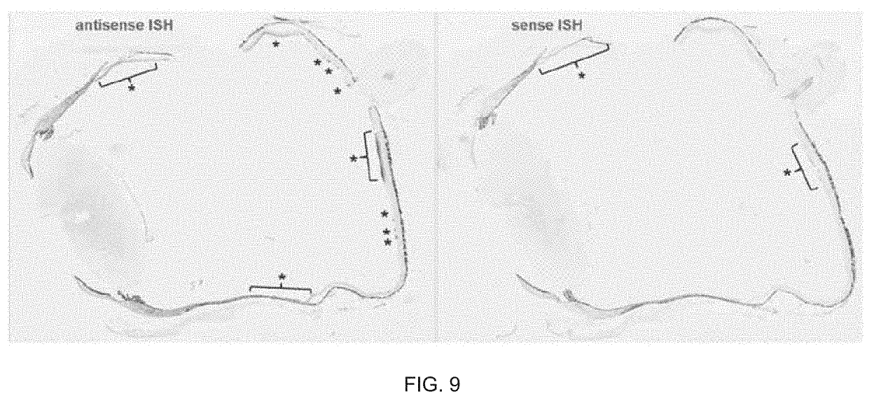

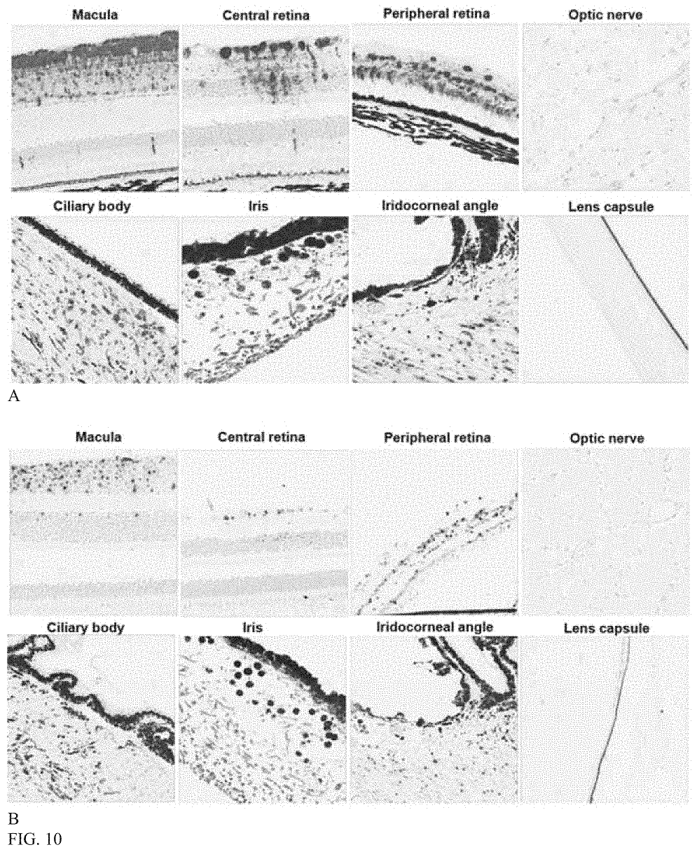

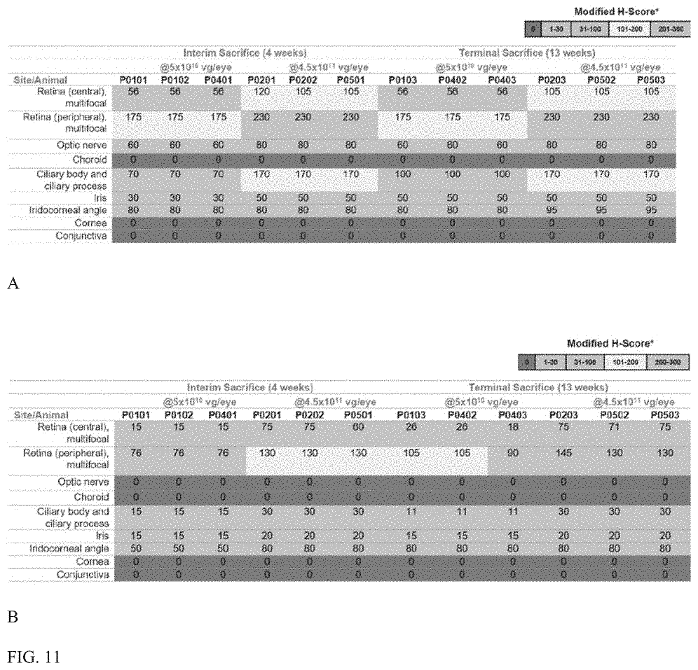

Having established the specificity of the probes, the exemplary vector nucleic acid distribution in the study eye tissues was further characterized. The eye sections used for the ISH assays showed moderate levels of PPIB and negative for DapB (a bacterial gene) mRNA signal validating the RNA integrity and ISH assay procedure. There was no exemplary vector nucleic acid signal in uninjected or in archived control/vehicle control macaque eyes. The antisense ISH signal was present in multifocal areas of central retina with abundant signal in macula, but more uniformly distributed in peripheral retina. Signal was also present in ciliary body, iris, iridocorneal angle, lens capsule and optic nerve. Localization pattern of the sense ISH signal was similar to that of the antisense ISH (FIG. 9). Within retina, ISH signal was abundant within retinal ganglia cells. Mild to moderate levels of the exemplary vector nucleic acids signal was noted in nerve fiber layer, inner plexiform layer, inner nuclear layer and occasionally within outer nuclear layer, photoreceptors and blood vessel walls (FIG. 10). There were no ISH signal in cornea, choroid and conjunctiva. Both the antisense and sense ISH signal was higher in high dose group (4.5×10¹¹ vg/eye) compared to low dose group (5×10¹⁰ vg/eye), but there was no significant differences in the ISH signal between interim (4 weeks post dose) and terminal (13 weeks post dose) sacrificed animals (FIG. 11).

Pretreatment with RNase eliminated antisense cytoplasmic signal and revealed intranuclear signal within retina (predominantly within ganglion cells), ciliary body, and iridocorneal angle consistent with transduction of the exemplary vector. DNase pretreatment retained intranuclear and intracytoplasmic ISH signal in retina, optic nerve, ciliary body, iris and iridocorneal angle, consistent with expression of MW-Opsin. ISH signal in lens capsule and nerve fiber layer were removed by DNase, but not by RNase treatment suggesting that the exemplary vector binds to these membranes.

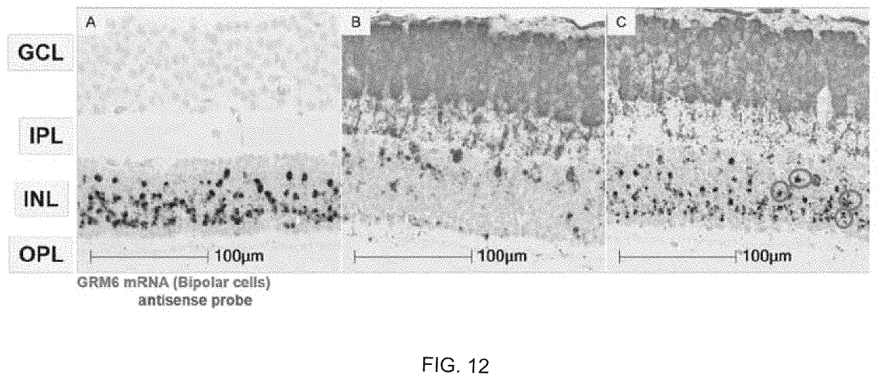

To investigate whether the exemplary vector nucleic localizes in bipolar cells of inner nuclear layer, dual antisense and GRM6 (a maker of ON bipolar cells) ISH experiments were performed. GRM6 mRNA signal was localized only in the inner nuclear layers, consistent with the published reports (Nakajima et al. 1993; Kim et al. 2008). As shown in FIG. 12, few antisense signal was colocalized with the GRM6 ISH positive bipolar cells, particularly evident in macula.

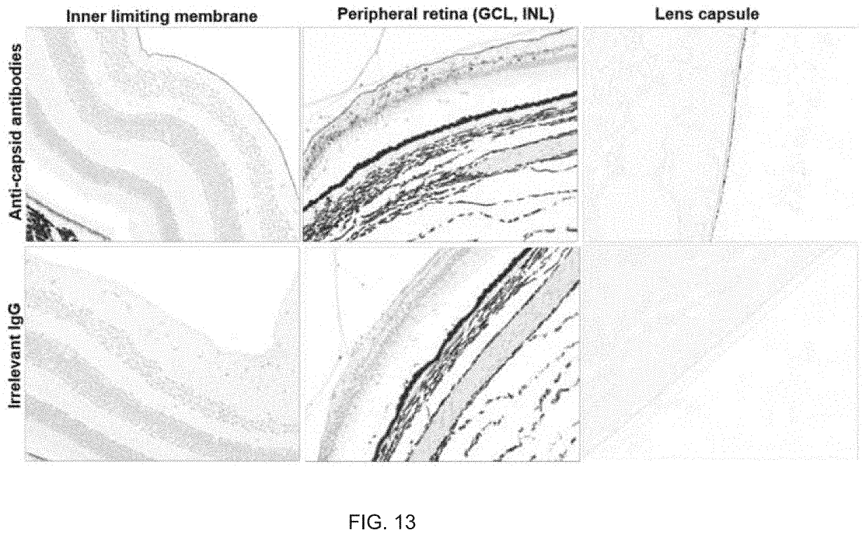

To further demonstrate the exemplary vector transduction in the study eyes, localization of AAV capsid proteins with anti-AAV VP1/VP2/VP3 antibodies was performed. As expected, positive control cynomolgus eye tissues showed a strong nuclear immunostaining in the outer nuclear layer. In the positive control study animal, AAV capsid immunostaining was noted within nuclei of ganglion cells and outer nuclear layer of peripheral retina, inner limiting membrane of retina and posterior lens capsule (FIG. 13). Nuclear staining of ganglion cells and outer nuclear layer suggest exemplary vector transduction. Presence of capsid proteins in lens capsule and inner limiting membrane suggests vector adherence to these membranes, consistent with exemplary vector nucleic acid detection. The absence of immunostaining in other areas of eye or in other study animals was attributed to the tissue fixation effects as Modified Davidson's reagent is not compatible to a variety of antibodies (Chidlow et al. 2011).

Polyclonal antibodies to human medium (a.k.a. green) and long (a.k.a. red) wave cone opsins were used to demonstrate expression of MW-Opsin, the exemplary transgene product. Positive control cells (with human MW-cone opsin transgene) were immunopositive, but negative control cells (with ChrimsonR-eGFP transgene) were immunonegative for opsin demonstrating the specificity of the antibodies.

Photoreceptors in both central (with uniform distribution) and peripheral retina (with multifocal distribution) showed strong staining in eye sections from the study animals, suggesting that the polyclonal antibodies cross-react with the cynomolgus macaque cone opsin. Except photoreceptors, the uninjected eyes of the exemplary vector dosed animals and the control animals did not show immunostaining in other areas of the eye sections. Minimal (grade 1) to mild (grade 2) immunostaining for MW-Opsin was noted in central and/or peripheral retina in all animals (Table 5) confirming the translation of transgene in the vector transduced cells.

TABLE 5
Opsin Protein Immunostaining in Retina of Exemplary Vector Injected Cynomolgus
Macaques

| | Interim Sacrifice (4 weeks) | | | | | | Terminal Sacrifice (13 weeks) | | | | | |
|---|---|---|---|---|---|---|---|---|---|---|---|---|
| | 5 × 10¹⁰ vg/eye | | | 4.5 × 10¹¹ vg/eye | | | 5 × 10¹⁰ vg/eye | | | 4.5 × 10¹¹ vg/eye | | |
| Animal/Site | P0101 | P0102 | P0401 | P0201 | P0202 | P0501 | P0103 | P0402 | P0403 | P0302 | P0502 | P0503 |
| Retina, central | 1 | 1 | 1 | 1 | 1 | 1 | 0 | 0 | 1 | 1 | 1 | 1 |
| Retina, | 2 | 2 | 1 | 2 | 2 | 1 | 1 | 1 | 1 | 2 | 2 | 2 |
| peripheral | | | | | | | | | | | | |
| Optic Nerve | 0 | 0 | 0 | 0 | 0 | 0 | 0 | 0 | 0 | 0 | 0 | 0 |
| Choroid | 0 | 0 | 0 | 0 | 0 | 0 | 0 | 0 | 0 | 0 | 0 | 0 |
| Ciliary Body | 0 | 0 | 0 | 1 | 1 | 0 | 1 | 1 | 0 | 1 | 1 | 1 |
| and Ciliary | | | | | | | | | | | | |
| Processes | | | | | | | | | | | | |
| Iris | 0 | 0 | 0 | 0 | 0 | 0 | 0 | 0 | 0 | 0 | 0 | 0 |
| Iridocorneal | 0 | 0 | 0 | 0 | 0 | 0 | 0 | 0 | 0 | 0 | 0 | 0 |
| Angle | | | | | | | | | | | | |

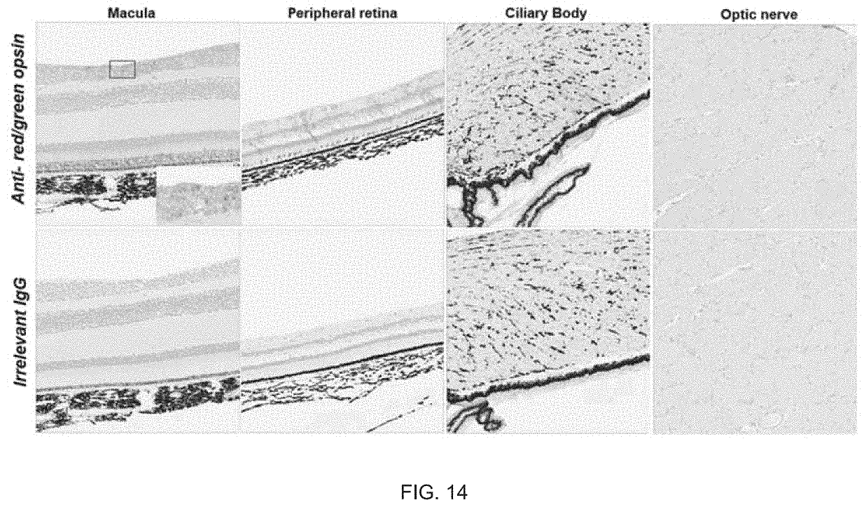

Within central retina, immunostaining was detected in ganglion cells and nerve fiber layer, whereas, peripheral retina showed immunostaining in multifocal areas spanning the entire thickness of neuroretina, resembling a Muller cell pattern (FIG. 14), consistent with exemplary vector nucleic acid localization. In addition, ciliary body and nonpigmented epithelium of ciliary process showed minimal immunostaining. There was no staining for MW-opsin in optic nerve, iris, iridocorneal angle, choroid, lens, conjunctiva and cornea. The staining intensity of MW-opsin immunostaining was lower than that of the exemplary vector nucleic acid localization and may be attributed to tissue fixation effects with Modified Davidson's reagent.

In situ hybridization with antisense and sense probes was used to demonstrate exemplary vector transduction in sections of paraffin embedded Modified Davidson's fixed cynomolgus eyes. Exemplary vector sequence was detected in test article injected eyes, but not in control animals of all interim and terminal necropsy animals. The ISH signal was localized in multifocal areas of central retina, particularly in macula. The ISH signal showed a uniform distribution in peripheral retina, ciliary body, iris, iridocorneal angle, lens capsule and optic nerve. As expected, vector transduction was abundant in the retinal ganglion cells. However, other cell types in retina (inner nuclear layer), ciliary body and iris are also transduced by the exemplary vector.

The exemplary vector nucleic acid signal was higher with high dose group (4.5×10¹¹ vg/eye) compared to low dose group (5×10¹⁰ vg/eye). However, it is interesting to note that there was no difference between the exemplary vector nucleic signal between interim and terminal sacrifice animals. This observation suggests that the vector persists for up to 13 weeks in the eye. In addition, the presence of vector nucleic acid in iridocorneal angle may suggests a clearance route of the vector.

Dual in situ hybridization experiments showed localization of exemplary vector nucleic acids in few GRM6 positive ON bipolar cells and that are particular evident in macula. In central and peripheral retina, the exemplary vector nucleic acid distribution appear to have a Mueller cell pattern.

Immunohistochemistry demonstrated the presence of AAV intranuclear capsid proteins in retina confirming transduction of the exemplary vector. Furthermore, MW-Opsin immunostaining was demonstrated in central and/or peripheral retina and ciliary body confirming translation of trangene product of the exemplary vector. Reduced sensitivity of capsid protein or MW-opsin detection in eye sections was attributed to antigen loss with the use of Modified Davidson's fixative (Chidlow et al. 2011).

What is claimed is:

1. A recombinant expression vector comprising a first ITR polynucleotide sequence, a promoter polynucleotide sequence operatively linked to a polynucleotide sequence encoding a medium-wavelength cone opsin (MW-opsin) transgene, a polyA polynucleotide sequence, an intron polynucleotide sequence, and a second ITR polynucleotide sequence.

2. A recombinant expression vector comprising a first ITR polynucleotide sequence, a promoter polynucleotide sequence operatively linked to a polynucleotide sequence encoding a medium-wavelength cone opsin (MW-opsin) transgene, an enhancer polynucleotide sequence, a polyA polynucleotide sequence, and a second ITR polynucleotide sequence.

3. A recombinant expression vector comprising a first ITR polynucleotide sequence, a promoter polynucleotide sequence operatively linked to a polynucleotide sequence encoding a medium-wavelength cone opsin (MW-opsin) transgene, an enhancer polynucleotide sequence, a polyA polynucleotide sequence, an intron polynucleotide sequence, and a second ITR polynucleotide sequence.

4. The recombinant expression vector of any of claims 1-3, wherein the first ITR polynucleotide sequence comprises the sequence of SEQ ID NO: 1.

5. The recombinant expression vector of any of claims 1-4, wherein the promoter polynucleotide sequence comprises the sequence of SEQ ID NO: 2.

6. The recombinant expression vector of any of claims 1-5, wherein the polynucleotide sequence encoding a codon-optimized MW-opsin transgene comprises a sequence that is 85% identical to the sequence of SEQ ID NO: 3.

7. The recombinant expression vector of any of claims 1-5, wherein the polynucleotide sequence encoding a MW-opsin transgene comprises a sequence that is 90% identical to the sequence of SEQ ID NO: 3.

8. The recombinant expression vector of any of claims 1-5, wherein the polynucleotide encoding a MW-opsin transgene comprises the sequence of SEQ ID NO: 3.

9. The recombinant expression vector of any of claims 2 and 4-8, wherein the enhancer polynucleotide sequence comprises the sequence of SEQ ID NO: 4.

10. The recombinant expression vector of any of claims 1-9, wherein the polyA polynucleotide sequence comprises the sequence of SEQ ID NO: 5.

11. The recombinant expression vector of any of claims 3-10, wherein the intron polynucleotide sequence comprises the sequence of SEQ ID NO: 6.

12. The recombinant expression vector of any of claims 1-11, wherein the second ITR polynucleotide sequence comprises the sequence of SEQ ID NO: 7.

13. The recombinant expression vector of any of claims 1-12, wherein the recombinant expression vector further comprises a polynucleotide sequence conferring resistance to an antibiotic.

14. The recombinant expression vector of claim 13, wherein the antibiotic is kanamycin.

15. The recombinant expression vector of any of claims 1-14, wherein the recombinant expression vector comprises the sequence of SEQ ID NO: 8.

16. The recombinant expression vector of any of claims 1-15, wherein the recombinant expression vector is a recombinant viral vector.

17. The recombinant viral vector of claim 16, wherein the recombinant viral vector is an adeno-associated viral vector, a lentiviral vector, a herpes simplex vector, or a retroviral vector.

18. The recombinant viral vector of claim 17, wherein the recombinant viral vector is an adeno-associated viral vector.

19. The recombinant viral vector of claim 18, wherein the recombinant viral vector is AAV2.

20. The recombinant viral vector of claim 18, wherein the recombinant adeno-associate viral vector comprises a nucleotide sequence encoding a variant capsid polypeptide that confers increased infectivity of a retinal cell and/or confers increased ability to cross the inner limiting membrane, as compared to a wild-type adeno-associated viral capsid.

21. The recombinant expression vector of any of claim 19 or 20, wherein the recombinant expression vector comprises the sequence of SEQ ID NO: 9.

22. The recombinant viral vector of claim 21, wherein the variant capsid polypeptide has a sequence selected from the group consisting of SEQ ID NOs: 10-197.

23. The recombinant viral vector of claim 20, wherein the variant capsid polypeptide has a sequence selected from the group consisting of SEQ ID NOs: 10-20.

24. The recombinant viral vector of claim 20, wherein the variant capsid polypeptide has the sequence of SEQ ID NO: 14.

25. The recombinant viral vector of claim 20, wherein the variant capsid polypeptide has the sequence of SEQ ID NO: 15.

26. The recombinant viral vector of claim 20, wherein the variant capsid polypeptide has the sequence of SEQ ID NO: 16.

27. A method of restoring or enhancing visual function in an individual, the method comprising administering to the individual the recombinant expression vector of any of claims 1-26, wherein said administering provides for expression of the MW-opsin transgene in a retinal cell in the individual and restoration or enhancement of visual function.

28. The method of claim 27, wherein expression of the MW-opsin transgene in the retinal cell provides for patterned vision and image recognition by the individual.

29. The method of claim 28, wherein the image recognition is of a static image or a pattern.

30. The method of claim 28, wherein the image recognition is of a moving image or a pattern.

31. The method of any of claims 27-30, wherein the expression of the MW-opsin transgene in the retinal cell provides for distinguishing between an image comprising a vertical line and an image comprising a horizontal line in a spatial pattern discrimination assay.

32. The method of any of claims 27-30, wherein the expression of the MW-opsin transgene in the retinal cell provides for distinguishing between an image comprising a static line and an image comprising a moving line in a spatial pattern discrimination assay.

33. The method of any of claims 27-30, wherein the expression of the MW-opsin transgene in the retinal cell provides for distinguishing between flashing light and constant light in a temporal light pattern assay.

34. The method of any of claims 27-30, wherein the expression of the MW-opsin transgene in the retinal cell provides for recognizing an image at a light intensity of from about 10⁴ W/cm² to about 10 W/cm² in an image recognition assay.

35. The method of any of claims 27-30, wherein the expression of the MW-opsin transgene in the retinal cell provides for distinguishing between an area with white light and an area without white light in a light avoidance assay.

36. The method of any of claims 27-30, wherein the expression of the MW-opsin transgene in the retinal cell provides for image recognition at a light intensity that is at least 10-fold lower than the light intensity required to provide for image recognition by an individual expressing a channelrhodopsin polypeptide in a retinal cell.

37. The method of any one of claims 27-30, wherein the expression of the MW-opsin transgene in the retinal cell provides for kinetics that are at least 2-fold faster than the kinetics conferred on a retinal cell by a rhodopsin polypeptide.

38. The method of any of claims 27-37, wherein said administering is via intraocular injection.

39. The method of any of claims 27-37, wherein said administering is via intravitreal injection.

40. The method of any of claims 27-37, wherein said administering is via subretinal injection.

41. The method of any of claims 27-37, wherein the individual has an ocular disease selected from retinitis pigmentosa, macular degeneration, geographic atrophy, age-related macular degeneration, retinoschisis, Leber's Congenital Amaurosis, cone rod dystrophies, Bardet Biedl syndrome, choroideremia, Usher syndrome, Stargardt disease, and Bietti crystalline dystrophy.

42. The method of any of claims 27-37, wherein the individual has experience retinal detachment or photoreceptor loss due to trauma, head injury, or as a complication of another disease.

43. A pharmaceutical composition comprising:
a) the recombinant expression vector of any of claims 1-26; and
b) a pharmaceutically acceptable excipient.

44. The pharmaceutical composition of claim 43, wherein the pharmaceutically acceptable excipient comprises saline.

45. The pharmaceutical composition of any of claims 43-44, wherein the composition is sterile.

46. The recombinant expression vector of any one of claims 1-26 or the pharmaceutical composition of any of claims 43-45 for use in treating a subject in need thereof.

47. The recombinant expression vector of any one of claims 1-26 or the pharmaceutical composition of any of claims 43-45 for use in restoring or enhancing visual function in a subject.

48. The use of the recombinant expression vector of any one of claims 1-26 or the pharmaceutical composition of any of claims 43-45, for the manufacture of a medicament for treating ocular disease.

49. The recombinant expression vector of any one of claims 1-26 or the pharmaceutical composition of any of claims 43-45, for use in restoring or enhancing visual function.

50. The recombinant expression vector of any one of claims 1-26 or the pharmaceutical composition of any of claims 43-45, for use in the treatment of ocular disease.

51. A host cell comprising the recombinant expression vector of any of claims 1-26.

52. A method of making the recombinant expression vector of any one of claims 1-26, said method comprising culturing the host cell of claim 51, lysing the cultured host cells, and extracting and purifying the recombinant expression vector from said lysed cultured host cells.

53. A method of making the pharmaceutical composition of any one of claims 43-45, said method comprising culturing the host cell of claim 51, collecting the supernatant of the cultured host cells, concentrating and purifying recombinant viral vectors from the collected supernatant, and adding pharmaceutically acceptable excipients to the purified recombinant viral vectors.

54. A method of treating an ocular disease selected from retinitis pigmentosa, macular degeneration, geographic atrophy, age-related macular degeneration, retinoschisis, Leber's Congenital Amaurosis, cone rod dystrophies, Bardet Biedl syndrome, choroideremia, Usher syndrome, Stargardt disease, or Bietti crystalline dystrophya, said method comprising administering a therapeutically effective amount of the recombinant expression vector of any of claims 1-26 or the pharmaceutical composition of claims 43-45 to a subject in need thereof.

55. The method of claim 54, wherein the ocular disease is retinitis pigmentosa.

56. The method of claim 54, wherein the ocular disease is geographic atrophy.

57. The recombinant viral vector of claim 20, wherein the variant capsid polypeptide has a sequence selected from the group consisting of SEQ ID NOs: 168-170.

58. The recombinant viral vector of claim 20, wherein the variant capsid polypeptide has the sequence of SEQ ID NO: 168.

59. The recombinant viral vector of claim 20, wherein the variant capsid polypeptide has the sequence of SEQ ID NO: 169.

60. The recombinant viral vector of claim 20, wherein the variant capsid polypeptide has the sequence of SEQ ID NO: 170.


SEQUENCE LISTING (206 sequences)

SEQ ID NO: 1 - DNA, 141 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 1677 bp, Artificial Sequence
  Description of Artificial Sequence Synthetic polynucleotide
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggccccccccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgttgcgcgctgccttccccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 3 - DNA, 1095 bp, Artificial Sequence
  Description of Artificial Sequence Synthetic polynucleotide
atggcccaacaatggtcccttcaacgactcgccggtagacacccacaggactcctacgaagattcgacccagtcatccattttcacttacaccaactccaactccactcgcggccccttcgagggcccgaattatcacattgcgccgagatgggtgtaccacctgactagcgtgtggatgatcttcgtcgtgatcgccagcgtgttcactaacggactggtgctggccgcgaccatgaagttcaagaagctgaggcaccctctgaactggattcttgtgaacctggccgtggccgacctggccgaaacagtgatcgcctcaaccatctccgtggtcaaccaggtctacggttactttgtgcttggacatcctatgtgcgtgctcgagggctacaccgtgtcgctgtgcgggatcactggattgtggtccctggccattatctcgtgggagcggtggatggttgtgtgcaagcccttcggcaacgtgcgcttcgatgcaaagctggctatcgtgggaatcgcgttttcctggatctgggccgccgtctggaccgctccccctattttcggttggtcccggtactggccccacgggctcaagacctcctgtggtcccgacgtgttcagcggatcgtcgtaccctggggtgcagtcctacatgattgtgctgatggtcacttgctgtatcacgccgctgtctattatcgtgctgtgctacctccaagtctggttggccatccgggctgtggccaaacagcagaaggagtccgagagcacccagaaagccgaaaaggaagtgacccggatggtcgtcgtgatggtgctggcattctgcttctgttggggcccgtacgctttctttgcctgctttgcggctgcgaacccgggctacccattccatcctctcatggccgccctcccggccttcttcgccaagtccgcgaccatctacaatcccgtgatctatgtgttcatgaaccggcagttccgcaactgcatcctgcaactcttcggaaagaaagtggacgacggatccgaactgtcgagcgcctcaaagaccgaagtcagctcggtgtcatccgtgagcccagcataa

SEQ ID NO: 4 - DNA, 590 bp, Artificial Sequence
  Description of Artificial Sequence Synthetic polynucleotide
aatcaacctctggattacaaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaggtctgctgagactcggggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 5 - DNA, 208 bp, Artificial Sequence
  Description of Artificial Sequence Synthetic polynucleotide
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 6 - DNA, 502 bp, Artificial Sequence
  Description of Artificial Sequence Synthetic polynucleotide
ctcatttcatctgtgacccctccactaccctttcttcctgattcttggaagcaaatccaagacatcacacccttccctctgtaaatctttactatgttcctctaggagaaaagggctcttctcaatacataaccacaagtcatcatcacaccgacaagtgtaacagtatttcctgaatagcttcaaatatcctagtagtgttcaaaaaatgtcatacgtattttcagtctgcttgaatcagggctcaaataaggtccacacattcagattgactgatatgccttttgactacctttgaatctagaggttccctttctatctccctgcaatttatttgtggaagcaagcaagtcgttcatgacgtagcctaacaggcccctctgacgttgttcattatgatttttctgtaaattggtagttgatctgaggatctggccagaggcaggttggatttgttggtgtgttttggcaaggagagtgtctcttttctggggtgttggca

SEQ ID NO: 7 - DNA, 141 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 8 - DNA, 4638 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatacgcatggagctagttattaatagtaatcaattacggggtcattagttcatagcccatatatggagttccgggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggccccccccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgttgcgcgctgccttccccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctggccaccatggcccaacaatggtcccttcaacgactcgccggtagacacccacaggactcctacgaagattcgacccagtcatccattttcacttacaccaactccaactccactcgcggccccttcgagggcccgaattatcacattgcgccgagatgggtgtaccacctgactagcgtgtggatgatcttcgtcgtgatcgccagcgtgttcactaacggactggtgctggccgcgaccatgaagttcaagaagctgaggcaccctctgaactggattcttgtgaacctggccgtggccgacctggccgaaacagtgatcgcctcaaccatctccgtggtcaaccaggtctacggttactttgtgcttggacatcctatgtgcgtgctcgagggctacaccgtgtcgctgtgcgggatcactggattgtggtccctggccattatctcgtgggagcggtggatggttgtgtgcaagcccttcggcaacgtgcgcttcgatgcaaagctggctatcgtgggaatcgcgttttcctggatctgggccgccgtctggaccgctccccctattttcggttggtcccggtactggccccacgggctcaagacctcctgtggtcccgacgtgttcagcggatcgtcgtaccctggggtgcagtcctacatgattgtgctgatggtcacttgctgtatcacgccgctgtctattatcgtgctgtgctacctccaagtctggttggccatccgggctgtggccaaacagcagaaggagtccgagagcacccagaaagccgaaaaggaagtgacccggatggtcgtcgtgatggtgctggcattctgcttctgttggggcccgtacgctttctttgcctgctttgcggctgcgaacccgggctacccattccatcctctcatggccgccctcccggccttcttcgccaagtccgcgaccatctacaatcccgtgatctatgtgttcatgaaccggcagttccgcaactgcatcctgcaactcttcggaaagaaagtggacgacggatccgaactgtcgagcgcctcaaagaccgaagtcagctcggtgtcatccgtgagcccagcataagcggaagcttccgtaatcaacctctggattacaaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaggtctgctgagactcggggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagcctgctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagaacgcgaccggtgatctgctcatttcatctgtgacccctccactaccctttcttcctgattcttggaagcaaatccaagacatcacacccttccctctgtaaatctttactatgttcctctaggagaaaagggctcttctcaatacataaccacaagtcatcatcacaccgacaagtgtaacagtatttcctgaatagcttcaaatatcctagtagtgttcaaaaaatgtcatacgtattttcagtctgcttgaatcagggctcaaataaggtccacacattcagattgactgatatgccttttgactacctttgaatctagaggttccctttctatctccctgcaatttatttgtggaagcaagcaagtcgttcatgacgtagcctaacaggcccctctgacgttgttcattatgatttttctgtaaattggtagttgatctgaggatctggccagaggcaggttggatttgttggtgtgttttggcaaggagagtgtctcttttctggggtgttggcatgtcgacctgattttgtataaccacttgcggtgatctagagcatggctatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 9 - DNA, 8270 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatacgcatggagctagttattaatagtaatcaattacggggtcattagttcatagcccatatatggagttccgggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggccccccccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgttgcgcgctgccttccccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctggccaccatggcccaacaatggtcccttcaacgactcgccggtagacacccacaggactcctacgaagattcgacccagtcatccattttcacttacaccaactccaactccactcgcggccccttcgagggcccgaattatcacattgcgccgagatgggtgtaccacctgactagcgtgtggatgatcttcgtcgtgatcgccagcgtgttcactaacggactggtgctggccgcgaccatgaagttcaagaagctgaggcaccctctgaactggattcttgtgaacctggccgtggccgacctggccgaaacagtgatcgcctcaaccatctccgtggtcaaccaggtctacggttactttgtgcttggacatcctatgtgcgtgctcgagggctacaccgtgtcgctgtgcgggatcactggattgtggtccctggccattatctcgtgggagcggtggatggttgtgtgcaagcccttcggcaacgtgcgcttcgatgcaaagctggctatcgtgggaatcgcgttttcctggatctgggccgccgtctggaccgctccccctattttcggttggtcccggtactggccccacgggctcaagacctcctgtggtcccgacgtgttcagcggatcgtcgtaccctggggtgcagtcctacatgattgtgctgatggtcacttgctgtatcacgccgctgtctattatcgtgctgtgctacctccaagtctggttggccatccgggctgtggccaaacagcagaaggagtccgagagcacccagaaagccgaaaaggaagtgacccggatggtcgtcgtgatggtgctggcattctgcttctgttggggcccgtacgctttctttgcctgctttgcggctgcgaacccgggctacccattccatcctctcatggccgccctcccggccttcttcgccaagtccgcgaccatctacaatcccgtgatctatgtgttcatgaaccggcagttccgcaactgcatcctgcaactcttcggaaagaaagtggacgacggatccgaactgtcgagcgcctcaaagaccgaagtcagctcggtgtcatccgtgagcccagcataagcggaagcttccgtaatcaacctctggattacaaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaggtctgctgagactcggggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagcctgctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagaacgcgaccggtgatctgctcatttcatctgtgacccctccactaccctttcttcctgattcttggaagcaaatccaagacatcacacccttccctctgtaaatctttactatgttcctctaggagaaaagggctcttctcaatacataaccacaagtcatcatcacaccgacaagtgtaacagtatttcctgaatagcttcaaatatcctagtagtgttcaaaaaatgtcatacgtattttcagtctgcttgaatcagggctcaaataaggtccacacattcagattgactgatatgccttttgactacctttgaatctagaggttccctttctatctccctgcaatttatttgtggaagcaagcaagtcgttcatgacgtagcctaacaggcccctctgacgttgttcattatgatttttctgtaaattggtagttgatctgaggatctggccagaggcaggttggatttgttggtgtgttttggcaaggagagtgtctcttttctggggtgttggcatgtcgacctgattttgtataaccacttgcggtgatctagagcatggctatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgcc

SEQ ID NO: 10 - protein, 9 aa, Artificial Sequence
  Description of Artificial Sequence Synthetic peptide
LAKDATKNA

SEQ ID NO: 11 - protein, 10 aa, Artificial Sequence
  Description of Artificial Sequence Synthetic peptide
PAHQDTTKNA

SEQ ID NO: 12 - protein, 10 aa, Artificial Sequence
  Description of Artificial Sequence Synthetic peptide
LAHQDTTKNA

SEQ ID NO: 13 - protein, 10 aa, Artificial Sequence
  Description of Artificial Sequence Synthetic peptide
LATTSQNKPA

SEQ ID NO: 14 - protein, 10 aa, Artificial Sequence
  Description of Artificial Sequence Synthetic peptide
LAISDQTKHA

SEQ ID NO: 15 - protein, 10 aa, Artificial Sequence
  Description of Artificial Sequence Synthetic peptide
IARGVAPSSA

SEQ ID NO: 16 - protein, 10 aa, Artificial Sequence
  Description of Artificial Sequence Synthetic peptide
LAPDSTTRSA

SEQ ID NO: 17 - protein, 10 aa, Artificial Sequence
  Description of Artificial Sequence Synthetic peptide
LAKGTELKPA

SEQ ID NO: 18 - protein, 10 aa, Artificial Sequence
  Description of Artificial Sequence Synthetic peptide
LAIIDATKNA

SEQ ID NO: 19 - protein, 10 aa, Artificial Sequence
  Description of Artificial Sequence Synthetic peptide
LAVDGAQRSA

SEQ ID NO: 20 - protein, 10 aa, Artificial Sequence
  Description of Artificial Sequence Synthetic peptide
PAPQDTTKKA

SEQ ID NO: 21 - protein, 10 aa, Artificial Sequence
  Description of Artificial Sequence Synthetic peptide
LPHQDTTKNA

SEQ ID NO: 22 - protein, 10 aa, Artificial Sequence
  Description of Artificial Sequence Synthetic peptide
LAKDATKTIA

SEQ ID NO: 23 - protein, 10 aa, Artificial Sequence
  Description of Artificial Sequence Synthetic peptide
LAKQQSASTA

SEQ ID NO: 24 - protein, 10 aa, Artificial Sequence
  Description of Artificial Sequence Synthetic peptide
LAKSDQSKPA

SEQ ID NO: 25 - protein, 10 aa, Artificial Sequence
  Description of Artificial Sequence Synthetic peptide
LSHQDTTKNA

SEQ ID NO: 26 - protein, 10 aa, Artificial Sequence
  Description of Artificial Sequence Synthetic peptide
LAANQPSKPA

SEQ ID NO: 27 - protein, 10 aa, Artificial Sequence
  Description of Artificial Sequence Synthetic peptide
LAVSDSTKAA

SEQ ID NO: 28 - protein, 11 aa, Artificial Sequence
  Description of Artificial Sequence Synthetic peptide
LAAQGTAKKPA

SEQ ID NO: 29 - protein, 10 aa, Artificial Sequence
  Description of Artificial Sequence Synthetic peptide
LAPDQTTRNA

SEQ ID NO: 30 - protein, 10 aa, Artificial Sequence
  Description of Artificial Sequence Synthetic peptide
LAASDSTKAA

SEQ ID NO: 31 - protein, 10 aa, Artificial Sequence
  Description of Artificial Sequence Synthetic peptide
LAPQDTTKNA

SEQ ID NO: 32 - protein, 10 aa, Artificial Sequence
  Description of Artificial Sequence Synthetic peptide
LAKADETRPA

SEQ ID NO: 33 - protein, 10 aa, Artificial Sequence
  Description of Artificial Sequence Synthetic peptide
LAHQDTAKNA

SEQ ID NO: 34 - protein, 10 aa, Artificial Sequence
  Description of Artificial Sequence Synthetic peptide
LAHQDTKKNA

SEQ ID NO: 35 - protein, 10 aa, Artificial Sequence
  Description of Artificial Sequence Synthetic peptide
LAHQDTTKHA

SEQ ID NO: 36 - protein, 10 aa, Artificial Sequence
  Description of Artificial Sequence Synthetic peptide
LAHQDTTKKA

SEQ ID NO: 37 - protein, 10 aa, Artificial Sequence
  Description of Artificial Sequence Synthetic peptide
LAHQDTTRNA

SEQ ID NO: 38 - protein, 9 aa, Artificial Sequence
  Description of Artificial Sequence Synthetic peptide
LAHQDTTNA

SEQ ID NO: 39 - protein, 10 aa, Artificial Sequence
  Description of Artificial Sequence Synthetic peptide
LAHQGTTKNA

SEQ ID NO: 40 - protein, 10 aa, Artificial Sequence
  Description of Artificial Sequence Synthetic peptide
LAHQVTTKNA

SEQ ID NO: 41 - protein, 10 aa, Artificial Sequence
  Description of Artificial Sequence Synthetic peptide
LAISDQSKPA

SEQ ID NO: 42 - protein, 8 aa, Artificial Sequence
  Description of Artificial Sequence Synthetic peptide
LADATKTA

SEQ ID NO: 43 - protein, 9 aa, Artificial Sequence
  Description of Artificial Sequence Synthetic peptide
LAKDTTKNA

SEQ ID NO: 44 - protein, 10 aa, Artificial Sequence
  Description of Artificial Sequence Synthetic peptide
LAKSDQSRPA

SEQ ID NO: 45 - protein, 10 aa, Artificial Sequence
  Description of Artificial Sequence Synthetic peptide
LAPQDTKKNA

SEQ ID NO: 46 - protein, 10 aa, Artificial Sequence
  Description of Artificial Sequence Synthetic peptide
LATSDSTKAA

SEQ ID NO: 47 - protein, 10 aa, Artificial Sequence
  Description of Artificial Sequence Synthetic peptide
LAVDGSQRSA

SEQ ID NO: 48 - protein, 10 aa, Artificial Sequence
  Description of Artificial Sequence Synthetic peptide
LPISDQTKHA

SEQ ID NO: 49 - protein, 10 aa, Artificial Sequence
  Description of Artificial Sequence Synthetic peptide
LPKDATKTIA

SEQ ID NO: 50 - protein, 10 aa, Artificial Sequence
  Description of Artificial Sequence Synthetic peptide
LPPQDTTKNA

SEQ ID NO: 51 - protein, 10 aa, Artificial Sequence
  Description of Artificial Sequence Synthetic peptide
PAPQDTTKNA

SEQ ID NO: 52 - protein, 10 aa, Artificial Sequence
  Description of Artificial Sequence Synthetic peptide
QAHQDTTKNA

SEQ ID NO: 53 - protein, 10 aa, Artificial Sequence
  Description of Artificial Sequence Synthetic peptide
LAHETSPRPA

SEQ ID NO: 54 - protein, 10 aa, Artificial Sequence
  Description of Artificial Sequence Synthetic peptide
LAKSTSTAPA

SEQ ID NO: 55 - protein, 10 aa, Artificial Sequence
  Description of Artificial Sequence Synthetic peptide
LADQDTTKNA

SEQ ID NO: 56 - protein, 10 aa, Artificial Sequence
  Description of Artificial Sequence Synthetic peptide
LAESDQSKPA

SEQ ID NO: 57 - protein, 10 aa, Artificial Sequence
  Description of Artificial Sequence Synthetic peptide
LAHKDTTKNA

SEQ ID NO: 58 - protein, 9 aa, Artificial Sequence
  Description of Artificial Sequence Synthetic peptide
LAHKTQQKM

SEQ ID NO: 59 - protein, 10 aa, Artificial Sequence
  Description of Artificial Sequence Synthetic peptide
LAHQDTTENA

SEQ ID NO: 60 - protein, 10 aa, Artificial Sequence
  Description of Artificial Sequence Synthetic peptide
LAHQDTTINA

SEQ ID NO: 61 - protein, 10 aa, Artificial Sequence
  Description of Artificial Sequence Synthetic peptide
LAHQDTTKKT

SEQ ID NO: 62 - protein, 10 aa, Artificial Sequence
  Description of Artificial Sequence Synthetic peptide
LAHQDTTKND

SEQ ID NO: 63 - protein, 10 aa, Artificial Sequence
  Description of Artificial Sequence Synthetic peptide
LAHQDTTKNT

SEQ ID NO: 64 - protein, 10 aa, Artificial Sequence
  Description of Artificial Sequence Synthetic peptide
LAHQDTTKNV

SEQ ID NO: 65 - protein, 10 aa, Artificial Sequence
  Description of Artificial Sequence Synthetic peptide
LAHQDTTKTM

SEQ ID NO: 66 - protein, 10 aa, Artificial Sequence
  Description of Artificial Sequence Synthetic peptide
LAHQNTTKNA

SEQ ID NO: 67 - protein, 10 aa, Artificial Sequence
  Description of Artificial Sequence Synthetic peptide
LAHRDTTKNA

SEQ ID NO: 68 - protein, 10 aa, Artificial Sequence
  Description of Artificial Sequence Synthetic peptide
LAISDQTNHA

SEQ ID NO: 69 - protein, 10 aa, Artificial Sequence
  Description of Artificial Sequence Synthetic peptide
LAKQKSASTA

SEQ ID NO: 70 - protein, 10 aa, Artificial Sequence
  Description of Artificial Sequence Synthetic peptide
LAKSDQCKPA

SEQ ID NO: 71 - protein, 10 aa, Artificial Sequence
  Description of Artificial Sequence Synthetic peptide
LAKSDQSKPD

SEQ ID NO: 72 - protein, 10 aa, Artificial Sequence
  Description of Artificial Sequence Synthetic peptide
LAKSDQSNPA

SEQ ID NO: 73 - protein, 10 aa, Artificial Sequence
  Description of Artificial Sequence Synthetic peptide
LAKSYQSKPA

SEQ ID NO: 74 - protein, 10 aa, Artificial Sequence
  Description of Artificial Sequence Synthetic peptide
LANQDTTKNA

SEQ ID NO: 75 - protein, 10 aa, Artificial Sequence
  Description of Artificial Sequence Synthetic peptide
LAPQNTTKNA

SEQ ID NO: 76 - protein, 10 aa, Artificial Sequence
  Description of Artificial Sequence Synthetic peptide
LAPSSIQKPA

SEQ ID NO: 77 - protein, 10 aa, Artificial Sequence
  Description of Artificial Sequence Synthetic peptide
LAQQDTTKNA

SEQ ID NO: 78 - protein, 10 aa, Artificial Sequence
  Description of Artificial Sequence Synthetic peptide
LAYQDTTKNA

SEQ ID NO: 79 - protein, 10 aa, Artificial Sequence
  Description of Artificial Sequence Synthetic peptide
LDHQDTTKNA

SEQ ID NO: 80 - protein, 10 aa, Artificial Sequence
  Description of Artificial Sequence Synthetic peptide
LDHQDTTKSA

SEQ ID NO: 81 - protein, 10 aa, Artificial Sequence
  Description of Artificial Sequence Synthetic peptide
LGHQDTTKNA

SEQ ID NO: 82 - protein, 10 aa, Artificial Sequence
  Description of Artificial Sequence Synthetic peptide
LPHQDTTKND

SEQ ID NO: 83 - protein, 10 aa, Artificial Sequence
  Description of Artificial Sequence Synthetic peptide
LPHQDTTKNT

SEQ ID NO: 84 - protein, 10 aa, Artificial Sequence
  Description of Artificial Sequence Synthetic peptide
LPHQDTTNNA

SEQ ID NO: 85 - protein, 10 aa, Artificial Sequence
  Description of Artificial Sequence Synthetic peptide
LTHQDTTKNA

SEQ ID NO: 86 - protein, 10 aa, Artificial Sequence
  Description of Artificial Sequence Synthetic peptide
LTKDATKTIA

SEQ ID NO: 87 - protein, 10 aa, Artificial Sequence
  Description of Artificial Sequence Synthetic peptide
LTPQDTTKNA

SEQ ID NO: 88 - protein, 10 aa, Artificial Sequence
  Description of Artificial Sequence Synthetic peptide
LVHQDTTKNA

SEQ ID NO: 89 - protein, 6 aa, Artificial Sequence
  Description of Artificial Sequence Synthetic peptide
KDATKN

SEQ ID NO: 90 - protein, 7 aa, Artificial Sequence
  Description of Artificial Sequence Synthetic peptide
HQDTTKN

SEQ ID NO: 91 - protein, 7 aa, Artificial Sequence
  Description of Artificial Sequence Synthetic peptide
HQDTTKN

SEQ ID NO: 92 - protein, 7 aa, Artificial Sequence
  Description of Artificial Sequence Synthetic peptide
TTSQNKP

SEQ ID NO: 93 - protein, 7 aa, Artificial Sequence
  Description of Artificial Sequence Synthetic peptide
ISDQTKH

SEQ ID NO: 94 - protein, 7 aa, Artificial Sequence
  Description of Artificial Sequence Synthetic peptide
RGVAPSS

SEQ ID NO: 95 - protein, 7 aa, Artificial Sequence
  Description of Artificial Sequence Synthetic peptide
PDSTTRS

SEQ ID NO: 96 - protein, 7 aa, Artificial Sequence
  Description of Artificial Sequence Synthetic peptide
KGTELKP

SEQ ID NO: 97 - protein, 7 aa, Artificial Sequence
  Description of Artificial Sequence Synthetic peptide
IIDATKN

SEQ ID NO: 98 - protein, 7 aa, Artificial Sequence
  Description of Artificial Sequence Synthetic peptide
VDGAQRS

SEQ ID NO: 99 - protein, 7 aa, Artificial Sequence
  Description of Artificial Sequence Synthetic peptide
PQDTTKK

SEQ ID NO: 100 - protein, 7 aa, Artificial Sequence
  Description of Artificial Sequence Synthetic peptide
HQDTTKN

SEQ ID NO: 101 - protein, 7 aa, Artificial Sequence
  Description of Artificial Sequence Synthetic peptide
KDATKTI

SEQ ID NO: 102 - protein, 7 aa, Artificial Sequence
  Description of Artificial Sequence Synthetic peptide
KQQSAST

SEQ ID NO: 103 - protein, 7 aa, Artificial Sequence
  Description of Artificial Sequence Synthetic peptide
KSDQSKP

SEQ ID NO: 104 - protein, 7 aa, Artificial Sequence
  Description of Artificial Sequence Synthetic peptide
HQDTTKN

SEQ ID NO: 105 - protein, 7 aa, Artificial Sequence
  Description of Artificial Sequence Synthetic peptide
ANQPSKP

SEQ ID NO: 106 - protein, 7 aa, Artificial Sequence
  Description of Artificial Sequence Synthetic peptide
VSDSTKA

SEQ ID NO: 107 - protein, 8 aa, Artificial Sequence
  Description of Artificial Sequence Synthetic peptide
AQGTAKKP

SEQ ID NO: 108 - protein, 7 aa, Artificial Sequence
  Description of Artificial Sequence Synthetic peptide
PDQTTRN

SEQ ID NO: 109 - protein, 7 aa, Artificial Sequence
  Description of Artificial Sequence Synthetic peptide
ASDSTKA

SEQ ID NO: 110 - protein, 7 aa, Artificial Sequence
  Description of Artificial Sequence Synthetic peptide
PQDTTKN

SEQ ID NO: 111 - protein, 7 aa, Artificial Sequence
  Description of Artificial Sequence Synthetic peptide
KADETRP

SEQ ID NO: 112 - protein, 7 aa, Artificial Sequence
  Description of Artificial Sequence Synthetic peptide
HQDTAKN

SEQ ID NO: 113 - protein, 7 aa, Artificial Sequence
  Description of Artificial Sequence Synthetic peptide
HQDTKKN

SEQ ID NO: 114 - protein, 7 aa, Artificial Sequence
  Description of Artificial Sequence Synthetic peptide
HQDTTKH

SEQ ID NO: 115 - protein, 7 aa, Artificial Sequence
  Description of Artificial Sequence Synthetic peptide
HQDTTKK

SEQ ID NO: 116 - protein, 7 aa, Artificial Sequence
  Description of Artificial Sequence Synthetic peptide
HQDTTRN

SEQ ID NO: 117 - protein, 6 aa, Artificial Sequence
  Description of Artificial Sequence Synthetic peptide
HQDTTN

SEQ ID NO: 118 - protein, 7 aa, Artificial Sequence
  Description of Artificial Sequence Synthetic peptide
HQGTTKN

SEQ ID NO: 119 - protein, 7 aa, Artificial Sequence
  Description of Artificial Sequence Synthetic peptide
HQVTTKN

SEQ ID NO: 120 - protein, 7 aa, Artificial Sequence
  Description of Artificial Sequence Synthetic peptide
ISDQSKP

SEQ ID NO: 121 - protein, 5 aa, Artificial Sequence
  Description of Artificial Sequence Synthetic peptide
DATKT

SEQ ID NO: 122 - protein, 6 aa, Artificial Sequence
  Description of Artificial Sequence Synthetic peptide
KDTTKN

SEQ ID NO: 123 - protein, 7 aa, Artificial Sequence
  Description of Artificial Sequence Synthetic peptide
KSDQSRP

SEQ ID NO: 124 - protein, 7 aa, Artificial Sequence
  Description of Artificial Sequence Synthetic peptide
PQDTKKN

SEQ ID NO: 125 - protein, 7 aa, Artificial Sequence
  Description of Artificial Sequence Synthetic peptide
TSDSTKA

SEQ ID NO: 126 - protein, 7 aa, Artificial Sequence
  Description of Artificial Sequence Synthetic peptide
VDGSQRS

SEQ ID NO: 127 - protein, 7 aa, Artificial Sequence
  Description of Artificial Sequence Synthetic peptide
ISDQTKH

SEQ ID NO: 128 - protein, 7 aa, Artificial Sequence
  Description of Artificial Sequence Synthetic peptide
KDATKTI

SEQ ID NO: 129 - protein, 7 aa, Artificial Sequence
  Description of Artificial Sequence Synthetic peptide
PQDTTKN

SEQ ID NO: 130 - protein, 7 aa, Artificial Sequence
  Description of Artificial Sequence Synthetic peptide
PQDTTKN

SEQ ID NO: 131 - protein, 7 aa, Artificial Sequence
  Description of Artificial Sequence Synthetic peptide
HQDTTKN

SEQ ID NO: 132 - protein, 7 aa, Artificial Sequence
  Description of Artificial Sequence Synthetic peptide
HETSPRP

SEQ ID NO: 133 - protein, 7 aa, Artificial Sequence
  Description of Artificial Sequence Synthetic peptide
KSTSTAP

SEQ ID NO: 134 - protein, 7 aa, Artificial Sequence
  Description of Artificial Sequence Synthetic peptide
DQDTTKN

SEQ ID NO: 135 - protein, 7 aa, Artificial Sequence
  Description of Artificial Sequence Synthetic peptide
ESDQSKP

SEQ ID NO: 136 - protein, 7 aa, Artificial Sequence
  Description of Artificial Sequence Synthetic peptide
HKDTTKN

SEQ ID NO: 137 - protein, 6 aa, Artificial Sequence
  Description of Artificial Sequence Synthetic peptide
HKTQQK

SEQ ID NO: 138 - protein, 7 aa, Artificial Sequence
  Description of Artificial Sequence Synthetic peptide
HQDTTEN

SEQ ID NO: 139 - protein, 7 aa, Artificial Sequence
  Description of Artificial Sequence Synthetic peptide
HQDTTIN

SEQ ID NO: 140 - protein, 7 aa, Artificial Sequence
  Description of Artificial Sequence Synthetic peptide
HQDTTKK

SEQ ID NO: 141 - protein, 7 aa, Artificial Sequence
  Description of Artificial Sequence Synthetic peptide
HQDTTKN

SEQ ID NO: 142 - protein, 7 aa, Artificial Sequence
  Description of Artificial Sequence Synthetic peptide
HQDTTKN

SEQ ID NO: 143 - protein, 7 aa, Artificial Sequence
  Description of Artificial Sequence Synthetic peptide
HQDTTKN

SEQ ID NO: 144 - protein, 7 aa, Artificial Sequence
  Description of Artificial Sequence Synthetic peptide
HQDTTKT

SEQ ID NO: 145 - protein, 7 aa, Artificial Sequence
  Description of Artificial Sequence Synthetic peptide
HQNTTKN

SEQ ID NO: 146 - protein, 7 aa, Artificial Sequence
  Description of Artificial Sequence Synthetic peptide
HRDTTKN

SEQ ID NO: 147 - protein, 7 aa, Artificial Sequence
  Description of Artificial Sequence Synthetic peptide
ISDQTNH

SEQ ID NO: 148 - protein, 7 aa, Artificial Sequence
  Description of Artificial Sequence Synthetic peptide
KQKSAST

SEQ ID NO: 149 - protein, 7 aa, Artificial Sequence
  Description of Artificial Sequence Synthetic peptide
KSDQCKP

SEQ ID NO: 150 - protein, 7 aa, Artificial Sequence
  Description of Artificial Sequence Synthetic peptide
KSDQSKP

SEQ ID NO: 151 - protein, 7 aa, Artificial Sequence
  Description of Artificial Sequence Synthetic peptide
KSDQSNP

SEQ ID NO: 152 - protein, 7 aa, Artificial Sequence
  Description of Artificial Sequence Synthetic peptide
KSYQSKP

SEQ ID NO: 153 - protein, 7 aa, Artificial Sequence
  Description of Artificial Sequence Synthetic peptide
NQDTTKN

SEQ ID NO: 154 - protein, 7 aa, Artificial Sequence
  Description of Artificial Sequence Synthetic peptide
PQNTTKN

SEQ ID NO: 155 - protein, 7 aa, Artificial Sequence
  Description of Artificial Sequence Synthetic peptide
PSSIQKP

SEQ ID NO: 156 - protein, 7 aa, Artificial Sequence
  Description of Artificial Sequence Synthetic peptide
QQDTTKN

SEQ ID NO: 157 - protein, 7 aa, Artificial Sequence
  Description of Artificial Sequence Synthetic peptide
YQDTTKN

SEQ ID NO: 158 - protein, 7 aa, Artificial Sequence
  Description of Artificial Sequence Synthetic peptide
HQDTTKN

SEQ ID NO: 159 - protein, 7 aa, Artificial Sequence
  Description of Artificial Sequence Synthetic peptide
HQDTTKS

SEQ ID NO: 160 - protein, 7 aa, Artificial Sequence
  Description of Artificial Sequence Synthetic peptide
HQDTTKN

SEQ ID NO: 161 - protein, 7 aa, Artificial Sequence
  Description of Artificial Sequence Synthetic peptide
HQDTTKN

SEQ ID NO: 162 - protein, 7 aa, Artificial Sequence
  Description of Artificial Sequence Synthetic peptide
HQDTTKN

SEQ ID NO: 163 - protein, 7 aa, Artificial Sequence
  Description of Artificial Sequence Synthetic peptide
HQDTTNN

SEQ ID NO: 164 - protein, 7 aa, Artificial Sequence
  Description of Artificial Sequence Synthetic peptide
HQDTTKN

SEQ ID NO: 165 - protein, 7 aa, Artificial Sequence
  Description of Artificial Sequence Synthetic peptide
KDATKTI

SEQ ID NO: 166 - protein, 7 aa, Artificial Sequence
  Description of Artificial Sequence Synthetic peptide
PQDTTKN

SEQ ID NO: 167 - protein, 7 aa, Artificial Sequence
  Description of Artificial Sequence Synthetic peptide
HQDTTKN

SEQ ID NO: 168 - protein, 7 aa, Artificial Sequence
  Description of Artificial Sequence Synthetic peptide
HQDTTKN

SEQ ID NO: 169 - protein, 7 aa, Artificial Sequence
  Description of Artificial Sequence Synthetic peptide
LGETTRA

SEQ ID NO: 170 - protein, 7 aa, Artificial Sequence
  Description of Artificial Sequence Synthetic peptide
HQDTTRP

SEQ ID NO: 171 - protein, 7 aa, Artificial Sequence
  Description of Artificial Sequence Synthetic peptide
RQDTTKN

SEQ ID NO: 172 - protein, 7 aa, Artificial Sequence
  Description of Artificial Sequence Synthetic peptide
HQDSTKN

SEQ ID NO: 173 - protein, 7 aa, Artificial Sequence
  Description of Artificial Sequence Synthetic peptide
HQDATKN

SEQ ID NO: 174 - protein, 7 aa, Artificial Sequence
  Description of Artificial Sequence Synthetic peptide
HQDTKKP

SEQ ID NO: 175 - protein, 7 aa, Artificial Sequence
  Description of Artificial Sequence Synthetic peptide
LSETTRP

SEQ ID NO: 176 - protein, 7 aa, Artificial Sequence
  Description of Artificial Sequence Synthetic peptide
HQDTTKK

SEQ ID NO: 177 - protein, 7 aa, Artificial Sequence
  Description of Artificial Sequence Synthetic peptide
LGEATRP

SEQ ID NO: 178 - protein, 7 aa, Artificial Sequence
  Description of Artificial Sequence Synthetic peptide
LGETTRT

SEQ ID NO: 179 - protein, 7 aa, Artificial Sequence
  Description of Artificial Sequence Synthetic peptide
LSEATRP

SEQ ID NO: 180 - protein, 7 aa, Artificial Sequence
  Description of Artificial Sequence Synthetic peptide
KDETKNS

SEQ ID NO: 181 - protein, 7 aa, Artificial Sequence
  Description of Artificial Sequence Synthetic peptide
LGETTKP

SEQ ID NO: 182 - protein, 7 aa, Artificial Sequence
  Description of Artificial Sequence Synthetic peptide
HQATTKN

SEQ ID NO: 183 - protein, 10 aa, Artificial Sequence
  Description of Artificial Sequence Synthetic peptide
LAHQDTTKNS

SEQ ID NO: 184 - protein, 10 aa, Artificial Sequence
  Description of Artificial Sequence Synthetic peptide
LALGETTRAA

SEQ ID NO: 185 - protein, 10 aa, Artificial Sequence
  Description of Artificial Sequence Synthetic peptide
LAHQDTTRPA

SEQ ID NO: 186 - protein, 10 aa, Artificial Sequence
  Description of Artificial Sequence Synthetic peptide
LARQDTTKNA

SEQ ID NO: 187 - protein, 10 aa, Artificial Sequence
  Description of Artificial Sequence Synthetic peptide
LAHQDSTKNA

SEQ ID NO: 188 - protein, 10 aa, Artificial Sequence
  Description of Artificial Sequence Synthetic peptide
LAHQDATKNA

SEQ ID NO: 189 - protein, 10 aa, Artificial Sequence
  Description of Artificial Sequence Synthetic peptide
LAHQDTKKPA

SEQ ID NO: 190 - protein, 9 aa, Artificial Sequence
  Description of Artificial Sequence Synthetic peptide
ILSETTRPA

SEQ ID NO: 191 - protein, 10 aa, Artificial Sequence
  Description of Artificial Sequence Synthetic peptide
LAHQDTTKKC

SEQ ID NO: 192 - protein, 10 aa, Artificial Sequence
  Description of Artificial Sequence Synthetic peptide
LALGEATRPA

SEQ ID NO: 193 - protein, 10 aa, Artificial Sequence
  Description of Artificial Sequence Synthetic peptide
LALGETTRTA

SEQ ID NO: 194 - protein, 10 aa, Artificial Sequence
  Description of Artificial Sequence Synthetic peptide
LALSEATRPA

SEQ ID NO: 195 - protein, 10 aa, Artificial Sequence
  Description of Artificial Sequence Synthetic peptide
LAKDETKNSA

SEQ ID NO: 196 - protein, 10 aa, Artificial Sequence
  Description of Artificial Sequence Synthetic peptide
LALGETTKPA

SEQ ID NO: 197 - protein, 10 aa, Artificial Sequence
  Description of Artificial Sequence Synthetic peptide
LAHQATTKNA

SEQ ID NO: 198 - DNA, 20 bp, Artificial Sequence
  Description of Artificial Sequence Synthetic primer
gtctgaatccacccagaagg

SEQ ID NO: 199 - DNA, 18 bp, Artificial Sequence
  Description of Artificial Sequence Synthetic primer
tgcgaagaaggcgtatgg

SEQ ID NO: 200 - DNA, 24 bp, Artificial Sequence
  Description of Artificial Sequence Synthetic probe
tgatggtcctggcattctgcttct

SEQ ID NO: 201 - DNA, 22 bp, Artificial Sequence
  Description of Artificial Sequence Synthetic primer
tcacttacaccaactccaactc

SEQ ID NO: 202 - DNA, 22 bp, Artificial Sequence
  Description of Artificial Sequence Synthetic primer
gaagatcatccacacgctagtc

SEQ ID NO: 203 - DNA, 24 bp, Artificial Sequence
  Description of Artificial Sequence Synthetic probe
ttatcacattgcgccgagatgggt

SEQ ID NO: 204 - DNA, 20 bp, Artificial Sequence
  Description of Artificial Sequence Synthetic primer
gtccgcgaccatctacaatc

SEQ ID NO: 205 - DNA, 21 bp, Artificial Sequence
  Description of Artificial Sequence Synthetic primer
atccgtcgtccactttctttc

SEQ ID NO: 206 - DNA, 24 bp, Artificial Sequence
  Description of Artificial Sequence Synthetic probe
cgaagagttgcaggatgcagttgc